(19) United States
(12) Patent Application Publication
(10) Pub. No.: US 2010/0056460 A1
(43) Pub. Date: Mar. 4, 2010

(54) COMBINATION OF ORGANIC COMPOUNDS

(76) Inventors: Mohammed A. Ali, Paterson, NJ (US); Margaret F. Prescott, Morris Plains, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/993,127

(22) PCT Filed: Jun. 28, 2006
(86) PCT No.: PCT/US2006/025865

§ 371 Date: Dec. 19, 2007

Related U.S. Application Data
(60) Provisional application No. 60/696,252, filed on Jul. 1, 2005.

(51) Int. Cl. A61K 38/05; A61P 3/10; A61P 9/04

(52) U.S. Cl. 514/19

(57) ABSTRACT

The invention relates to a combination, such as a combined preparation or a pharmaceutical composition, respectively, comprising a renin inhibitor, or a pharmaceutically acceptable salt thereof, and at least one therapeutic agent selected from the group consisting of

(a) an insulin secretion enhancer, or a pharmaceutically acceptable salt thereof; and
(b) an insulin sensitizer, or a pharmaceutically acceptable salt thereof.

The invention relates to a combination, such as a combined preparation or a pharmaceutical composition, respectively, comprising a renin inhibitor, or a pharmaceutically acceptable salt thereof, and at least one therapeutic agent selected from the group consisting of

(a) an insulin secretion enhancer, or a pharmaceutically acceptable salt thereof; and
(b) an insulin sensitizer, or a pharmaceutically acceptable salt thereof.

Furthermore, the present invention provides a method for the prevention of, delay the onset of or treatment of a disease or a condition modulated by the inhibition of renin activity and/or insulin secretion enhancer and/or insulin sensitizer, which method comprises administering to a warm-blooded animal, including man, in need thereof, a therapeutically effective amount of a combination comprising a renin inhibitor, or a pharmaceutically acceptable salt thereof, and at least one therapeutic agent selected from the group consisting of

(a) an insulin secretion enhancer, or a pharmaceutically acceptable salt thereof; and
(b) an insulin sensitizer, or a pharmaceutically acceptable salt thereof.

Diseases or a conditions modulated by the inhibition of renin activity and/or insulin secretion enhancer and/or insulin sensitizer include, but are not limited to, hypertension, congestive heart failure, diabetes, especially type 2 diabetes mellitus, mature onset diabetes of the young (MODY), diabetic retinopathy, macular degeneration, diabetic nephropathy, hypertensive or non-hypertensive nephropathy, IgA nephropathy, glomerulosclerosis, chronic renal failure, diabetic neuropathy, metabolic syndrome, cardiac syndrome X, atherosclerosis, coronary heart disease, angina pectoris, myocardial infarction, stroke, coronary and vascular restenosis, hyperglycemia, hyperinsulinaemia, hyperlipidaemia, hypertryglyceridemia, insulin resistance, impaired glucose metabolism (IGM), conditions of impaired glucose tolerance (IGT), IGM and/or IGT or increased inflammation in women with polycystic ovary syndrome or women with prior gestational diabetes, conditions of impaired fasting plasma glucose, obesity, diabetic retinopathy, macular degeneration, cataracts, foot ulcerations, endothelial dysfunction, impaired vascular compliance and obstructive sleep apnea. Preferably, the said combination may be used for the treatment of hypertension, including ISH, as well as congestive heart failure, metabolic syndrome, endothelial dysfunction, impaired vascular compliance, IGT, diabetes especially type II diabetes mellitus, hypertensive or non-hypertensive nephropathy, IgA nephropathy, as well as retardation or prolongation of the progression of prediabetes to diabetes.

The term “at least one therapeutic agent” shall mean that in addition to a renin inhibitor one or more, for example, two, furthermore three, active ingredients as specified according to the present invention can be combined.

The term “combination” of a renin inhibitor, or a pharmaceutically acceptable salt thereof, and at least one therapeutic agent selected from the group consisting of an insulin secretion enhancer, or a pharmaceutically acceptable salt thereof, and an insulin sensitizer, or a pharmaceutically acceptable salt thereof, means that the components can be administered together as a pharmaceutical composition or as part of the same, unitary dosage form. A combination also includes administering a renin inhibitor, or a pharmaceutically acceptable salt thereof, and at least one therapeutic agent selected from the group consisting of an insulin secretion enhancer, or a pharmaceutically acceptable salt thereof, and an insulin sensitizer, or a pharmaceutically acceptable salt thereof, each separately but as part of the same therapeutic regimen. The components, if administered separately, need not necessarily be administered at essentially the same time, although they can if so desired. Thus, a combination also refers, for example, administering a renin inhibitor, or a pharmaceutically acceptable salt thereof, and at least one therapeutic agent selected from the group consisting of an insulin secretion enhancer, or a pharmaceutically acceptable salt thereof, and an insulin sensitizer, or a pharmaceutically acceptable salt thereof, as separate dosages or dosage forms, but at the same time. A combination also includes separate administration at different times and in any order.

The term “prevention” refers to prophylactic administration to healthy patients to prevent the development of the conditions mentioned herein. Moreover, the term “prevention” means prophylactic administration to patients being in a pre-stage of the conditions to be treated.

The term “delay the onset of”, as used herein, refers to administration to patients being in a pre-stage of the condition to be treated in which patients with a pre-form of the corresponding condition is diagnosed.

The term “treatment” is understood the management and care of a patient for the purpose of combating the disease, condition or disorder.

The term “therapeutically effective amount” refers to an amount of a drug or a therapeutic agent that will elicit the desired biological or medical response of a tissue, system or an animal (including man) that is being sought by a researcher or clinician.

The term “warm-blooded animal or patient” are used interchangeably herein and include, but are not limited to, humans, dogs, cats, horses, pigs, cows, monkeys, rabbits, mice and laboratory animals. The preferred mammals are humans.

The term “pharmaceutically acceptable salt” refers to a non-toxic salt commonly used in the pharmaceutical industry which may be prepared according to methods well-known in the art.

“A disease or condition which may be modulated by the inhibition of renin activity” as defined in the present invention refers to hypertension, congestive heart failure, diabetes, especially type 2 diabetes mellitus, diabetic retinopathy, macular degeneration, diabetic nephropathy, hypertensive or non-hypertensive nephropathy and IgA nephropathy, glomerulosclerosis, renal failure, especially chronic renal failure, diabetic neuropathy, metabolic syndrome, cardiac syndrome X, atherosclerosis, coronary heart disease, angina pectoris, myocardial infarction, stroke, coronary and vascular restenosis, endothelial dysfunction, arterial stiffness, and the like.

“A disease or condition which may be modulated by an insulin secretion enhancer” as defined in the present invention refers to hyperglycemia, hyperinsulinaemia, hyperlipidaemia, hypertryglyceridemia, insulin resistance, impaired glucose metabolism, conditions of impaired glucose tolerance (IGT), impaired glucose metabolism (IGM), IGM and/or IGT in women with polycystic ovary syndrome or women with prior gestational diabetes, MODY, conditions of impaired fasting plasma glucose, obesity, diabetic retinopathy, macular degeneration, cataracts, diabetic nephropathy, hypertensive or non-hypertensive nephropathy and IgA nephropathy, glomerulosclerosis, diabetic neuropathy, erectile dysfunction, atherosclerosis, coronary heart disease, hypertension, angina pectoris, myocardial infarction, stroke, coronary and vascular restenosis, foot ulcerations, endothelial dysfunction, impaired vascular compliance and obstructive sleep apnea.

“A disease or condition that may be modulated by an insulin sensitizer” as defined in the present invention refers to hyperglycemia, hyperinsulinaemia, hyperlipidaemia, hypertryglyceridemia, insulin resistance, impaired glucose metabolism, conditions of impaired glucose tolerance (IGT), impaired glucose metabolism (IGM), IGM and/or IGT in women with polycystic ovary syndrome or women with prior gestational diabetes, MODY, conditions of impaired fasting plasma glucose, obesity, diabetic retinopathy, macular degeneration, cataracts, diabetic nephropathy, hypertensive or non-hypertensive nephropathy and IgA nephropathy, glomerulosclerosis, diabetic neuropathy, erectile dysfunction, atherosclerosis, coronary heart disease, hypertension, angina pectoris, myocardial infarction, stroke, coronary and vascular restenosis, foot ulcerations, endothelial dysfunction, impaired vascular compliance and obstructive sleep apnea.

The natural enzyme renin released from the kidneys cleaves angiotensinogen in the circulation to form the decapeptide called angiotensin I. This in turn is cleaved by angiotensin converting enzyme (ACE) in the lungs, kidneys and other organs to form the octapeptide called angiotensin II. Through its interaction with specific receptors on the surface of the target cells the octapeptide increases blood pressure both directly by arterial vasoconstriction and indirectly by liberating from the adrenal glands the sodium-ion-retaining hormone aldosterone, accompanied by an increase in extracellular fluid volume. It has been possible to identify receptor subtypes that are termed, e.g., AT₁- and AT₂-receptors. Inhibitors of the enzymatic activity of renin bring about a reduction in the formation of angiotensin I. As a result a smaller amount of angiotensin II is produced. The reduced concentration of that active peptide hormone is the direct cause of, e.g., the antihypertensive effect of renin inhibitors. Accordingly, renin inhibitors, or salts thereof, may be employed, e.g., as antihypertensives or for treating congestive heart failure.

The renin inhibitors to which the present invention applies are any of those having renin inhibitory activity in vivo and, therefore, pharmaceutical utility, e.g., as therapeutic agents for the prevention of, delay the onset of or treatment of hypertension, congestive heart failure, diabetes, especially type 2 diabetes mellitus, diabetic retinopathy, macular degeneration, diabetic nephropathy, hypertensive or non-hypertensive nephropathy and IgA nephropathy, glomerulosclerosis, renal failure, especially chronic renal failure, diabetic neuropathy, metabolic syndrome, cardiac syndrome X, atherosclerosis, coronary heart disease, angina pectoris, myocardial infarction, stroke, coronary and vascular restenosis, endothelial dysfunction, arterial stiffness, and the like.

In particular, the present invention relates to renin inhibitors disclosed in U.S. Pat. No. 5,559,111 and EP 678503 A; U.S. Pat. No. 6,197,959 and U.S. Pat. No. 6,376,672, the entire contents of which are incorporated herein by reference.

Suitable renin inhibitors include compounds having different structural features. For example, mention may be made of compounds which are selected from the group consisting of ditekiren (chemical name: [1S-[1R*,2R*,4R*(1R*,2R*)]]-1-[(1,1-dimethylethoxy)carbonyl]-L-proly I-L-phenylalanyl-N-[2-hydroxy-5-methyl-1-(2-methylpropyl)-4-[[[2-methyl-1-[[(2-pyridinylmrthyl)amino]carbonyl]butyl]amino]carbonyl]hexyl]-N-alfa-methyl-L-histidinamide); terlakiren (chemical name: [R-(R*,S*)]-N-(4-morpholinylcarbonyl)-L-phenylalanyl-N-[1-(cyclohexy Imethyl)-2-hydroxy-3-(1-methylethoxy)-3-oxopropyl]-S-methyl-L-cysteineamide); and zankiren (chemical name: [1S-[1R*[R*(R*)],2S*,3R*]]-N-[1-(cyclohexylmethyl)-2,3-dihydroxy-5-methylhexyl]-alfa-[[2-[[(4-methyl-1-piperazinyl)sulfonyl]methyl]-1-oxo-3-phenylpropyl]-amino]-4-thiazolepropanamide), preferably, in each case, the hydrochloride salt thereof.

Preferred renin inhibitor of the present invention include RO 66-1132 and RO 66-1168 of formulae (I) and (II)

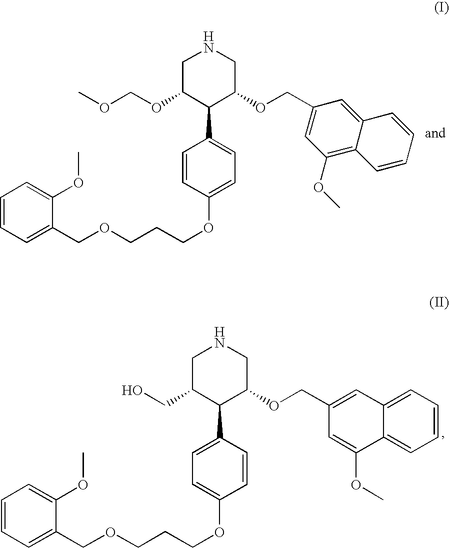

respectively, or a pharmaceutically acceptable salt thereof.

In particular, the present invention relates to a renin inhibitor which is a δ-amino-γ-hydroxy-ω-aryl-alkanoic acid amide derivative of the formula

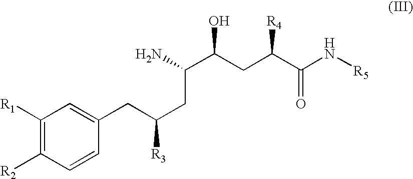

wherein R₁ is halogen, C₁₋₆halogenalkyl, C₁₋₆alkoxy-C₁₋₆alkyloxy or C₁₋₆alkoxy-C₁₋₆alkyl; R₂ is halogen, C₁₋₄alkyl or C₁₋₄alkoxy; R₃ and R₄ are independently branched C₃₋₆alkyl; and R₅ is cycloalkyl, C₁₋₆alkyl, C₁₋₆hydroxyalkyl, C₁₋₆alkoxy-C₁₋₆alkyl, C₁₋₆alkanoyloxy-C₁₋₆alkyl, C₁₋₆aminoalkyl, C₁₋₆alkylamino-C₁₋₆alkyl, C₁₋₆dialkylamino-C₁₋₆alkyl, C₁₋₆alkanoylamino-C₁₋₆alkyl, HO(O)C—C₁₋₆alkyl, C₁₋₆alkyl-O—(O)C—C₁₋₆alkyl, H₂N—C(O)—C₁₋₆alkyl, C₁₋₆alkyl-HN—C(O)—C₁₋₆alkyl or (C₁₋₆alkyl)₂N—C(O)—C₁₋₆alkyl; or a pharmaceutically acceptable salt thereof.

As an alkyl, R₁ may be linear or branched and preferably comprise 1 to 6 C atoms, especially 1 or 4 C atoms. Examples are methyl, ethyl, n- and i-propyl, n-, i- and t-butyl, pentyl and hexyl.

As a halogenalkyl, R₁ may be linear or branched and preferably comprise 1 to 4 C atoms, especially 1 or 2 C atoms. Examples are fluoromethyl, difluoromethyl, trifluoromethyl, chloromethyl, dichloromethyl, trichloromethyl, 2-chloroethyl and 2,2,2-trifluoroethyl.

As an alkoxy, R₁ and R₂ may be linear or branched and preferably comprise 1 to 4 C atoms. Examples are methoxy, ethoxy, n- and i-propyloxy, n-, i- and t-butyloxy, pentyloxy and hexyloxy.

As an alkoxyalkyl, R₁ may be linear or branched. The alkoxy group preferably comprises 1 to 4 and especially 1 or 2 C atoms, and the alkyl group preferably comprises 1 to 4 C atoms. Examples are methoxymethyl, 2-methoxyethyl, 3-methoxypropyl, 4-methoxybutyl, 5-methoxypentyl, 6-methoxyhexyl, ethoxymethyl, 2ethoxyethyl, 3-ethoxypropyl, 4-ethoxybutyl, 5-ethoxypentyl, 6-ethoxyhexyl, propyloxymethyl, butyloxymethyl, 2-propyloxyethyl and 2-butyloxyethyl.

As a C₁₋₆alkoxy-C₁₋₆alkyloxy, R₁ may be linear or branched. The alkoxy group preferably comprises 1 to 4 and especially 1 or 2 C atoms, and the alkyloxy group preferably comprises 1 to 4 C atoms. Examples are methoxymethyloxy, 2-methoxyethyloxy, 3-methoxypropyloxy, 4-methoxybutyloxy, 5-methoxypentyloxy, 6-methoxyhexyloxy, ethoxymethyloxy, 2-ethoxyethyloxy, 3-ethoxypropyloxy, 4-ethoxybutyloxy, 5-ethoxypentyloxy, 6-ethoxyhexyloxy, propyloxymethyloxy, butyloxymethyloxy, 2-propyloxyethyloxy and 2-butyloxyethyloxy.

In a preferred embodiment, R₁ is methoxy- or ethoxy-C₁₋₄alkyloxy, and R₂ is preferably methoxy or ethoxy. Particularly preferred are compounds of formula (III), wherein R₁ is 3-methoxypropyloxy and R₂ is methoxy.

As a branched alkyl, R₃ and R₄ preferably comprise 3 to 6 C atoms. Examples are i-propyl, i- and t-butyl, and branched isomers of pentyl and hexyl. In a preferred embodiment, R₃ and R₄ in compounds of formula (III) are in each case i-propyl.

As a cycloalkyl, R₅ may preferably comprise 3 to 8 ring-carbon atoms, 3 or 5 being especially preferred. Some examples are cyclopropyl, cyclobutyl, cyclopentyl, cyclohexyl and cyclooctyl. The cycloalkyl may optionally be substituted by one or more substituents, such as alkyl, halo, oxo, hydroxy, alkoxy, amino, alkylamino, dialkylamino, thiol, alkylthio, nitro, cyano, heterocyclyl and the like.

As an alkyl, R₅ may be linear or branched in the form of alkyl and preferably comprise 1 to 6 C atoms. Examples of alkyl are listed herein above. Methyl, ethyl, n- and i-propyl, n-, i- and t-butyl are preferred.

As a C₁₋₆hydroxyalkyl, R₅ may be linear or branched and preferably comprise 2 to 6 C atoms. Some examples are 2-hydroxyethyl, 2-hydroxypropyl, 3-hydroxypropyl, 2-, 3- or 4-hydroxybutyl, hydroxypentyl and hydroxyhexyl.

As a C₁₋₆alkoxy-C₁₋₆alkyl, R₅ may be linear or branched. The alkoxy group preferably comprises 1 to 4 C atoms and the alkyl group preferably 2 to 4 C atoms. Some examples are 2-methoxyethyl, 2-methoxypropyl, 3-methoxypropyl, 2-, 3- or 4-methoxybutyl, 2-ethoxyethyl, 2-ethoxypropyl, 3-ethoxypropyl, and 2-, 3- or 4-ethoxybutyl.

As a C₁₋₆alkanoyloxy-C₁₋₆alkyl, R₅ may be linear or branched. The alkanoyloxy group preferably comprises 1 to 4 C atoms and the alkyl group preferably 2 to 4 C atoms. Some examples are formyloxymethyl, formyloxyethyl, acetyloxyethyl, propionyloxyethyl and butyroyloxyethyl.

As a C₁₋₆-aminoalkyl, R₅ may be linear or branched and preferably comprise 2 to 4 C atoms. Some examples are 2-aminoethyl, 2- or 3-aminopropyl and 2-, 3- or 4-aminobutyl.

As C₁₋₆alkylamino-C₁₋₆alkyl and C₁₋₆dialkylamino-C₁₋₆alkyl, R₅ may be linear or branched. The alkylamino group preferably comprises C₁₋₄alkyl groups and the alkyl group has preferably 2 to 4 C atoms. Some examples are 2-methylaminoethyl, 2-dimethylaminoethyl, 2-ethylaminoethyl, 2-ethylaminoethyl, 3-methylaminopropyl, 3-dimethylaminopropyl, 4-methylaminobutyl and 4-dimethylaminobutyl.

As a HO(O)C—C₁₋₆alkyl, R₅ may be linear or branched and the alkyl group preferably comprises 2 to 4 C atoms. Some examples are carboxymethyl, carboxyethyl, carboxypropyl and carboxybutyl.

As a C₁₋₆alkyl-O—(O)C—C₁₋₆alkyl, R₅ may be linear or branched, and the alkyl groups preferably comprise independently of one another 1 to 4 C atoms. Some examples are methoxycarbonylmethyl, 2-methoxycarbonylethyl, 3-methoxycarbonylpropyl, 4-methoxy-carbonylbutyl, ethoxycarbonylmethyl, 2-ethoxycarbonylethyl, 3-ethoxycarbonylpropyl, and 4-ethoxycarbonylbutyl.

As a H₂N—C(O)—C₁₋₆alkyl, R₅ may be linear or branched, and the alkyl group preferably comprises 2 to 6 C atoms. Some examples are carbamidomethyl, 2-carbamidoethyl, 2-carbamido-2,2-dimethylethyl, 2- or 3-carbamidopropyl, 2-, 3- or 4-carbamidobutyl, 3-carbamido-2-methylpropyl, 3-carbamido-1,2-dimethylpropyl, 3-carbamido-3-ethylpropyl, 3-carbamido-2,2-dimethylpropyl, 2-, 3-, 4- or 5-carbamidopentyl, 4-carbamido-3,3- or -2,2-dimethylbutyl. Preferably, R₅ is 2-carbamido-2,2-dimethylethyl.

Accordingly, preferred are δ-amino-γ-hydroxy-ω)-aryl-alkanoic acid amide derivatives of formula (III) having the formula

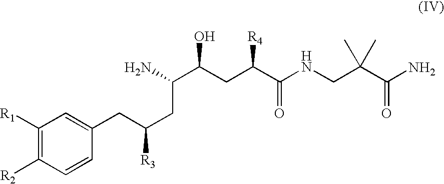

wherein R₁ is 3-methoxypropyloxy; R₂ is methoxy; and R₃ and R₄ are isopropyl; or a pharmaceutically acceptable salt thereof; chemically defined as 2(S),4(S),5(S),7(S)—N-(3-amino-2,2-dimethyl-3-oxopropyl)-2,7-di(1-methylethyl)-4-hydroxy-5-amino-8-[4-methoxy-3-(3-methoxy-propoxy)phenyl]-octanamide, also known as aliskiren.

The term “aliskiren”, if not defined specifically, is to be understood both as the free base and as a salt thereof, especially a pharmaceutically acceptable salt thereof, most preferably a hemi-fumarate salt thereof.

Insulin secretion enhancers are active ingredients that have the property to promote the secretion of insulin from pancreatic β-cells. Examples of insulin secretion enhancers for the purpose of the present invention are glucokinase activators (GKAs), which are compounds that have an activating effect on glucokinase.

Of the glucose phosphorylating enzymes present in the liver and the pancreatic β-cells, glucokinase (GK) is one of the most important. It plays a key role in regulating blood glucose homeostasis. In the β-cells, this hexokinase is considered responsible for glucose-stimulated insulin secretion and in the liver, it plays an important role in glucose uptake and glycogen synthesis.

GKA1 and GKA2 directly activate GK. They are chemically distinct and have potencies (EC₅₀) in the sub-micromolar range. GKA1 and GKA2 increase the affinity of GK for glucose by 4- and 11-fold, respectively. This action is principally responsible for the insulin secretion enhancing activity.

Binding of a GKA to an allosteric site on GK is known to result in a subtle change of structure, which seems to have a stabilizing effect on the closed form of GK. This change appears somewhat similar to activating mutations. Co-crystallization of GKAs with GK shows that these compounds bind to the allosteric pocket in GK. As this binding allosteric pocket is present only in GK and not in other hexokinases, GKA activation is limited to GK.

GKAs for the purpose of the present invention include, but are not limited to, 6-[(3-isobutoxy-5-isopropoxybenzoyl)amino]nicotinic acid (GKA1) of formula (V), 5-({3-isopropoxy-5-[2-(3-thienyl)ethoxy]benzoyl}amino)-1,3,4-thiadiazole-2-carboxylic acid (GKA2) of formula (VI), 2-(S)-Cyclohexyl-1-(R)-(4-methanesulfonyl-phenyl)-cyclopropanecarboxylic acid thiazol-2-ylamide (LY2121260) of formula (VII) and RO-28-1675 of formula (VIII)

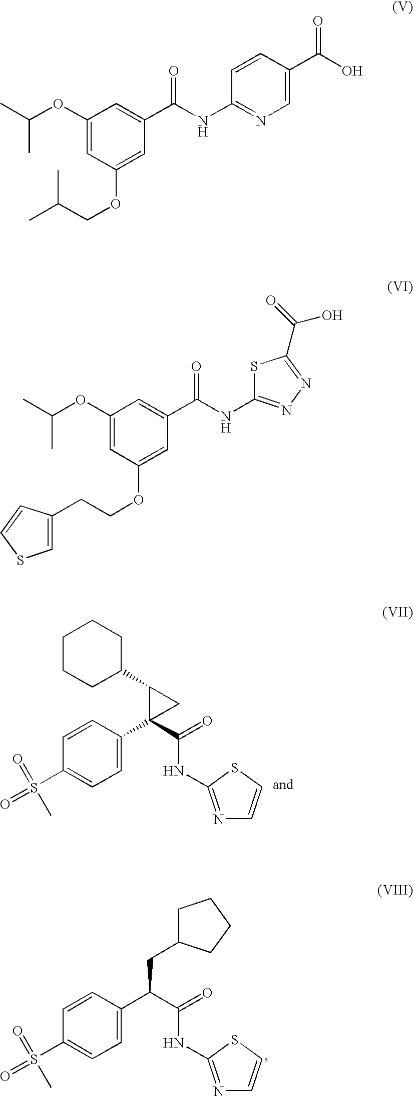

respectively, or a pharmaceutically acceptable salt thereof.

LY2121260 is known to alter the affinity of GK towards glucose and to significantly increase the velocity of the glucose phosphorylation reaction. As in the case of GKA1 and GKA2, the GK activating effect of this compound is on GK alone and not on other human hexokinases. RO-28-1675 is an R enantiomer and is known to significantly increase the enzymatic activity of human recombinant GK. It has also been shown to reverse the inhibitory action of human glucokinase regulatory protein.

In particular, the present invention relates to a GKA of the formula

R—NH-Q (IX)

wherein
(i) Q is a

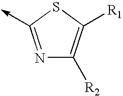

radical in which R₁ and R₂ are independently hydrogen or halogen; or
   Q is a

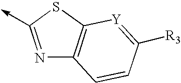

radical in which R₃ is hydrogen, halogen, alkyl, cycloalkyl, aryl, alkoxy, cycloalkoxy, aryloxy, alkylthio, cycloalkylthio, arylthio, acyl, sulfonyl, alkylamino, cycloalkylamino, arylamino, acylamino, sulfonamido or alkoxycarbonyl; Y is CH or nitrogen; and
R is a radical of the formula

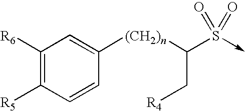

wherein
   R₄ is C₂₋₄alkyl, C₃₋₇cycloalkyl or C₅₋₇heterocycloalkyl;
   R₅ and R₆ are independently hydrogen, halogen, cyano, R₇, —C(O)R₇ or —S(O)₂R₇ wherein
      R₇ is —(CR₈R₉)ₘ—W—R₁₀ in which
         R₈ and R₉ are independently hydrogen or lower alkyl;
         W is a bond, O, S or —NR₁₁ in which
         R₁₁ is hydrogen or lower alkyl;
         R₁₀ is hydrogen, alkyl, cycloalkyl, aryl or heterocyclyl; or R₁₀ and R₁₁, combined, are alkylene which together with the nitrogen atom to which they are attached form a 5- to 7-membered ring;
         m is zero or an integer from 1 to 5;
      n is zero or an integer of 1 or 2;
         or an optical isomer thereof; or a pharmaceutically acceptable salt thereof; or
(ii) Q is a

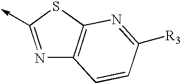

radical in which R₃ is hydrogen, halogen, alkyl, cycloalkyl, aryl, alkoxy, cycloalkoxy, aryloxy, alkylthio, cycloalkylthio, arylthio, acyl, sulfonyl, alkylamino, cycloalkylamino, arylamino, acylamino, sulfonamido or alkoxycarbonyl; and
   R is a radical of the formula

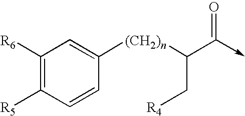

wherein
   R₄ is C₂₋₄alkyl, C₃₋₇cycloalkyl or C₅₋₇heterocycloalkyl;
   R₅ and R₆ are independently hydrogen, halogen, cyano, R₇, —C(O)R₇ or —S(O)₂R₇ wherein
      R₇ is —(CR₈R₉)ₘ—W—R₁₀ in which
         R₈ and R₉ are independently hydrogen or lower alkyl;
         W is a bond, O, S or —NR₁₁ in which
         R₁₁ is hydrogen or lower alkyl;
         R₁₀ is hydrogen, alkyl, cycloalkyl, aryl or heterocyclyl; or R₁₀ and R₁₁, combined, are alkylene which together with the nitrogen atom to which they are attached form a 5- to 7-membered ring;
         m is zero or an integer from 1 to 5;
   n is zero or an integer of 1 or 2;
      or an optical isomer thereof; or a pharmaceutically acceptable salt thereof; or
(iii) Q is

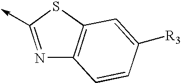

is a radical in which R₃ is hydrogen, halogen, alkyl, cycloalkyl, aryl, alkoxy, cycloalkoxy, aryloxy, alkylthio, cycloalkylthio, arylthio, acyl, sulfonyl, alkylamino, cycloalkylamino, arylamino, acylamino, sulfonamido or alkoxycarbonyl; and
   R is a radical of the formula

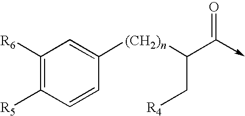

wherein
   R₄ is C₂₋₄alkyl, C₃₋₇cycloalkyl or C₅₋₇heterocycloalkyl;
   R₅ and R₆ are independently hydrogen, halogen, cyano, R₇, —C(O)R₇ or —S(O)₂R₇ wherein
      R₇ is —(CR₈R₉)ₘ—W—R₁₀ in which
         R₈ and R₉ are independently hydrogen or lower alkyl;
         W is a bond, O, S or —NR₁₁ in which
         R₁₁ is hydrogen or lower alkyl;
         R₁₀ is hydrogen, alkyl, cycloalkyl, aryl or heterocyclyl; or R₁₀ and R₁₁, combined, are alkylene which together with the nitrogen atom to which they are attached form a 5- to 7-membered ring;
         m is zero or an integer from 1 to 5;
   n is zero or an integer of 1 or 2;
      or an optical isomer thereof; or a pharmaceutically acceptable salt thereof; or
(iv) Q is a

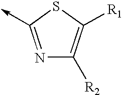

radical, wherein R₁ and R₂ are independently hydrogen or halogen; and
   R is a radical of the formula

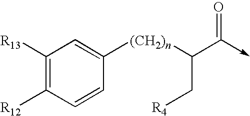

wherein
   R₄ is C₂₋₄alkyl, C₃₋₇cycloalkyl or C₅₋₇heterocycloalkyl;
   R₁₂ and R₁₃ are independently hydrogen, halogen, cyano, R₁₄, —C(O)R₁₄, or —S(O)₂R₁₄ wherein
      R₁₄ is —(CR₈R₉)ₘ, —W—R₅ in which
         R₈ and R₉ are independently hydrogen or lower alkyl;
         W is a bond, O, S or —NR₁₁ in which
         R₁₁ is hydrogen or lower alkyl;
         R₁₅ is cycloalkyl, aryl or heterocyclyl; or R₁₅ and R₁₁, combined, are alkylene which together with the nitrogen atom to which they are attached form a 5 to 7-membered ring;
         m is zero or an integer from 1 to 5;
   n is zero or an integer of 1 or 2;
      or an optical isomer thereof; or a pharmaceutically acceptable salt thereof.

In one embodiment, the compounds of formula (IX) have the formula

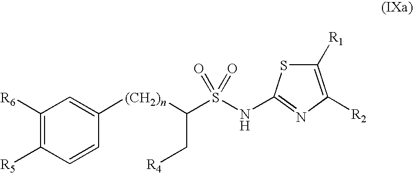

wherein
R₁ and R₂ are independently hydrogen or halogen;
R₄ is C₂₋₄alkyl, C₃₋₇cycloalkyl or C₅₋₇heterocycloalkyl;
R₅ and R₆ are independently hydrogen, halogen, cyano, R₇, —C(O)R₇ or —S(O)₂R₇ wherein
   R₇ is —(CR₈R₉)ₘ—W—R₁₀ in which
      R₈ and R₉ are independently hydrogen or lower alkyl;
      W is a bond, O, S or —NR₁₁ n which
         R₁₁ is hydrogen or lower alkyl;
      R₁₀ is hydrogen, alkyl, cycloalkyl, aryl or heterocyclyl; or R₁₀ and R₁₁, combined, are alkylene which together with the nitrogen atom to which they are attached form a 5- to 7-membered ring;
      m is zero or an integer from 1 to 5;
n is zero or an integer of 1 or 2;
   or an optical isomer thereof; or a pharmaceutically acceptable salt thereof.

Preferred are the compounds of formula (IXa), wherein
R₄ is cyclopentyl;
n is zero;
   or an optical isomer thereof; or a pharmaceutically acceptable salt thereof.

Other preferred compounds are the compounds of formula (IXa), wherein
R₄ is cyclopentyl;
n is zero;
R₆ is hydrogen or halogen;
R₅ is —S(O)₂R₇ wherein
   R₇ is —(CR₈R₉)ₘ—W—R₁₀ in which
      R₈ and R₉ are independently hydrogen or lower alkyl;
      W is a bond, O, S or —NR₁₁ in which
         R₁₁ is hydrogen or lower alkyl preferably hydrogen,
         most preferably W is a bond;
      R₁₀ is hydrogen, alkyl, cycloalkyl, aryl or heterocyclyl; or R₁₀ and R₁₁, combined, are alkylene which together with the nitrogen atom to which they are attached form a 5- to 7-membered ring;
      m is zero or an integer from 1 to 5;
         or an optical isomer thereof; or a pharmaceutically acceptable salt thereof.

In another embodiment, the compounds of formula (IX) have the formula

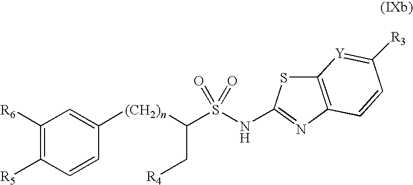

wherein
R₃ is hydrogen, halogen, alkyl, cycloalkyl, aryl, alkoxy, cycloalkoxy, aryloxy, alkylthio, cycloalkylthio, arylthio, acyl, sulfonyl, alkylamino, cycloalkylamino, arylamino, acylamino, sulfonamido or alkoxycarbonyl;
R₄ is C₂₋₄alkyl, C₃₋₇cycloalkyl or C₅₋₇heterocycloalkyl;
R₅ and R₆ are independently hydrogen, halogen, cyano, R₇, —C(O)R₇ or —S(O)₂R₇ wherein
   R₇ is —(CR₈R₉)ₘ—W—R₁₀ in which
      R₈ and R₉ are, independently, hydrogen or lower alkyl;
      W is a bond, O, S or —NR₁₁ in which R₁₁ is hydrogen or lower alkyl;
      R₁₀ is hydrogen, alkyl, cycloalkyl, aryl or heterocyclyl; or R₁₀ and R₁₁, combined, are alkylene which together with the nitrogen atom to which they are attached form a 5- to 7-membered ring;
      m is zero or an integer from 1 to 5;
Y is CH or nitrogen;
n is zero or an integer of 1 or 2;
   or an optical isomer thereof; or a pharmaceutically acceptable salt thereof.

Preferred are the compounds of formula (IXb), wherein
R₄ is cyclopentyl;
n is zero;
   or an optical isomer thereof; or a pharmaceutically acceptable salt thereof.

Other preferred compounds are the compounds of formula (IXb), wherein
R₄ is cyclopentyl;
n is zero;
R₆ is hydrogen or halogen preferably hydrogen;
R₅ is —S(O)₂R₇ wherein
   R₇ is —(CR₈R₉)ₘ—W—R₁₀ in which
      R₈ and R₉ are independently hydrogen or lower alkyl;
      W is a bond, O, S or —NR₁₁ in which
         R₁₁ is hydrogen or lower alkyl,
         most preferably W is a bond;
      R₁₀ is hydrogen, alkyl, cycloalkyl, aryl or heterocyclyl; or R₁₀ and R₁₁, combined, are alkylene which together with the nitrogen atom to which they are attached form a 5- to 7-membered ring;
      m is zero or an integer from 1 to 5;
         or an optical isomer thereof; or a pharmaceutically acceptable salt thereof.

Yet in another embodiment, the compounds of formula (IX) have the formula

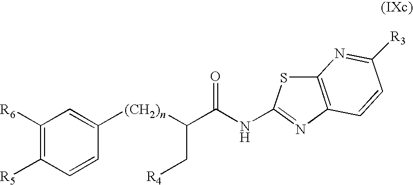

wherein
R₃ is hydrogen, halogen, alkyl, cycloalkyl, aryl, alkoxy, cycloalkoxy, aryloxy, alkylthio, cycloalkylthio, arylthio, acyl, sulfonyl, alkylamino, cycloalkylamino, arylamino, acylamino, sulfonamido or alkoxycarbonyl;
R₄ is C₂₋₄alkyl, C₃₋₇cycloalkyl or C₅₋₇heterocycloalkyl;
R₅ and R₆ are independently hydrogen, halogen, cyano, R₇, —C(O)R₇ or —S(O)₂R₇ wherein
   R₇ is —(CR₈R₉)ₘ—W—R₁₀ in which
      R₈ and R₉ are, independently, hydrogen or lower alkyl;
      W is a bond, O, S or —NR₁₁ in which
         R₁₁ is hydrogen or lower alkyl;
      R₁₀ is hydrogen, alkyl, cycloalkyl, aryl or heterocyclyl; or R₁₀ and R₁₁, combined, are alkylene which together with the nitrogen atom to which they are attached form a 5- to 7-membered ring;
      m is zero or an integer from 1 to 5;
n is zero or an integer of 1 or 2;
   or an optical isomer thereof; or a pharmaceutically acceptable salt thereof.

Preferred are the compounds of formula (IXc), wherein
R₄ is cyclopentyl;
n is zero;
   or an optical isomer thereof; or a pharmaceutically acceptable salt thereof.

Other preferred compounds are the compounds of formula (IXc), wherein
R₄ is cyclopentyl;
n is zero;
R₆ is hydrogen or halogen preferably hydrogen;
R₅ is —S(O)₂R₇ wherein
   R₇ is —(CR₈R₉)ₘ—W—R₁₀ in which
      R₈ and R₉ are independently hydrogen or lower alkyl;
      W is a bond, O, S or —NR₁₁ in which
         R₁₁ is hydrogen or lower alkyl,
         most preferably W is a bond;
      R₁₀ is hydrogen, alkyl, cycloalkyl, aryl or heterocyclyl; or R₁₀ and R₁₁, combined, are alkylene which together with the nitrogen atom to which they are attached form a 5- to 7-membered ring;
      m is zero or an integer from 1 to 5;
         or an optical isomer thereof; or a pharmaceutically acceptable salt thereof.

Other preferred compounds are the preferred compounds of formula (IXc) as described above, wherein R₃ is hydrogen or alkoxy.

Yet in another embodiment, the compounds of formula (IX) have the formula

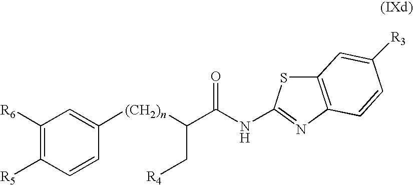

wherein
R₃ is hydrogen, halogen, alkyl, cycloalkyl, aryl, alkoxy, cycloalkoxy, aryloxy, alkylthio, cycloalkylthio, arylthio, acyl, sulfonyl, alkylamino, cycloalkylamino, arylamino, acylamino, sulfonamido or alkoxycarbonyl;
R₄ is C₂₋₄alkyl, C₃₋₇cycloalkyl or C₅₋₇heterocycloalkyl;
R₅ and R₆ are independently hydrogen, halogen, cyano, R₇, —C(O)R₇, or —S(O)₂R₇ wherein
   R₇ is —(CR₈R₉)ₘ—W—R₁₀ in which
      R₈ and R₉ are, independently, hydrogen or lower alkyl;
      W is a bond, O, S or —NR₁₁ in which
         R₁₁ is hydrogen or lower alkyl;
      R₁₀ is hydrogen, alkyl, cycloalkyl, aryl or heterocyclyl; or R₁₀ and R₁₁, combined, are alkylene which together with the nitrogen atom to which they are attached form a 5- to 7-membered ring;
      m is zero or an integer from 1 to 5;
n is zero or an integer of 1 or 2;
   or an optical isomer thereof; or a pharmaceutically acceptable salt thereof.

Preferred are the compounds of formula (IXd), wherein
R₄ is cyclopentyl;
n is zero;
   or an optical isomer thereof; or a pharmaceutically acceptable salt thereof.

Other preferred compounds are the compounds of formula (IXd), wherein
R₄ is cyclopentyl;
n is zero;
R₆ is hydrogen or halogen most preferably hydrogen;
R₅ is —S(O)₂R₇ wherein
   R₇ is —(CR₈R₉)ₘ—W—R₁₀ in which
      R₈ and R₉ are independently hydrogen or lower alkyl;
      W is a bond, O, S or —NR₁₁ in which
         R₁₁ is hydrogen or lower alkyl,
         most preferably W is a bond;
      R₁₀ is hydrogen, alkyl cycloalkyl, aryl or heterocyclyl; or R₁₀ and R₁₁, combined, are alkylene which together with the nitrogen atom to which they are attached form a 5- to 7-membered ring;
      m is zero or an integer from 1 to 5;
         or an optical isomer thereof; or a pharmaceutically acceptable salt thereof.

Yet in another embodiment, the compounds of formula (IX) have the formula

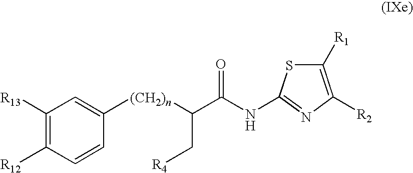

wherein
R₁ and R₂ are independently hydrogen or halogen;
R₄ is C₂₋₄alkyl, C₃₋₇cycloalkyl or C₅₋₇heterocycloalkyl;
R₁₂ and R₁₃ are independently hydrogen, halogen, cyano, R₁₄, —C(O)R₁₄, or —S(O)₂R₁₄ wherein
   R₁₄ is —(CR₈R₉)ₘ—W—R₁₅ in which
      R₈ and R₉ are, independently, hydrogen or lower alkyl;
      W is a bond, O, S or —NR₁₁ in which
         R₁₁ is hydrogen or lower alkyl;
      R₁₅ is cycloalkyl, aryl or heterocyclyl; or R₁₅ and R₁₁, combined, are alkylene which together with the nitrogen atom to which they are attached form a 5- to 7-membered ring;
      m is zero or an integer from 1 to 5;
n is zero or an integer of 1 or 2;
   or an optical isomer thereof; or a pharmaceutically acceptable salt thereof.

Preferred are the compounds of formula (IXe), wherein
R₄ is cyclopentyl;
n is zero;
   or an optical isomer thereof; or a pharmaceutically acceptable salt thereof.

Other preferred compounds are the compounds of formula (IXe), wherein
R₄ is cyclopentyl;
n is zero;
R₆ is hydrogen or halogen preferably hydrogen;
R₅ is —S(O)₂R₇ wherein
   R₇ is —(CR₈R₉)ₘ—W—R₁₀ in which
      R₈ and R₉ are independently hydrogen or lower alkyl;
      W is a bond, O, S or —NR₁₁ in which
         R₁₁ is hydrogen or lower alkyl,
         most preferably W is a bond;
      R₁₀ is hydrogen, alkyl, cycloalkyl, aryl or heterocyclyl; or R₁₀ and R₁₁ combined, are alkylene which together with the nitrogen atom to which they are attached form a 5- to 7-membered ring;
      m is zero or an integer from 1 to 5;
         or an optical isomer thereof; or a pharmaceutically acceptable salt thereof.

Methods of preparing the above compounds are disclosed in WO 2004050645, published Jun. 17, 2004, which is incorporated herein by reference in its entirety.

Insulin sensitizers are active ingredients that have the property to increase insulin sensitivity and elevate insulin signaling components.

Insulin sensitizers for the purpose of the present invention include, but are not limited to, inhibitors of stearoyl-CoA desaturase-1 (SCD-1), inhibitors of diacylglycerol acyltransferase 1 and 2 (DGAT1 and DGAT2) and inhibitors of phosphotyrosine phosphatase (PTPase).

Stearoyl-CoA desaturase (SCD) is an endoplasmic reticulum enzyme that is responsible for catalysis of the most important step in the biosynthesis of monounsaturated fatty acids from saturated fatty acids. The preferred substrates for this enzyme are palmitoyl- and stearoyl-CoA, which get converted into palmitoleoyl- and oleoyl-CoA respectively, the latter are the most abundant monounsaturated fatty acids in lipids, phospholipids, triglycerides, cholesteryl esters, wax esters and alkyldiacylglycerols. Additionally, monounsaturated fatty acids may also act as mediators of signal transduction, cellular differentiation and apoptosis. Therefore, regulation of the synthesis of these fatty acids through SCD has an effect on a wide spectrum of metabolic pathways including those involved in insulin signaling.

Mice with a targeted disruption of the SCD1 gene have been shown to demonstrate improved glucose tolerance compared with wild-type mice, despite lower fasting plasma insulin levels.

Inhibitors of SCD-1 include, but are not limited to, leptin, SCD specific antisense oligonucleotide inhibitors and SCD-1 specific inhibitors including, but not limited to, a compound of formula (Ia) as defined in WO 2005011653, claims 10 to 35; a compound of formula (IIa) as defined in WO 2005011654, claims 10 and 11; a compound of formula (IIb) as defined in WO 2005011654, claims 14 to 23; a compound of formula (III) as defined in WO 2005011654, claims 26 to 32, a compound of formula (IV) as defined in WO 2005011654, claims 35 to 41; a compound of the formula (V) as defined in WO 2005011654, claims 44 to 50; a compound of formula (VIa) as defined in WO 2005011654, claims 53 and 54; a compound of formula (VIb) as defined in WO 2005011654, claims 57 to 69; a compound of formula (II) as defined in WO 2005011655, claims 10 to 23; a compound of formula (III) as defined in WO 2005011655, claims 26 to 64; a compound of formula (IV) as defined in WO 2005011655, claims 67 to 80; a compound of formula (Va) as defined in WO 2005011655, claims 83 to 86; a compound of formula (Ia) as defined in WO 2005011655, claim 89; a compound of formula (IIa) as defined in WO 2005011656, claims 10 to 15; a compound of formula (IIb) as defined in WO 2005011656, claims 18 to 26; a compound of formula (III) as defined in WO 2005011656, claims 29 to 34; a compound of formula (IV) as defined in WO 2005011656, claims 37 to 48; a compound of formula (V) as defined in WO 2005011656, claims 51 to 58; a compound of formula (Ia) as defined in WO 2005011656, claims 61 to 68; a compound of formula (II) as defined in WO 2005011657, claims 10 to 26; a compound of formula (III) as defined in WO 2005011657, claims 29 to 35; a compound of formula (IV) as defined in WO 2005011657, claims 38 to 43; and a compound of formula (Ia) as defined in WO 2005011657, claims 46 to 49; in each case, the claims, the final products of the working examples and methods of making the same, and the pharmaceutical preparations thereof, are incorporated herein by reference. Preferred are the compounds disclosed in WO 2005011655.

Specific examples of SCD-1 specific inhibitors are:
1-Pentyl-3-{6-[4-(2-trifluoromethylbenzoyl)piperazin-1-yl]pyridazin-3-yl}urea;
1-Benzyl-3-{6-[4-(2-trifluoromethylbenzoyl)piperazin-1-yl]pyridazin-3-yl}urea;
1-(4-Fluorophenyl)-3-{6-[4-(2-trifluoromethylbenzoyl)piperazin-1-yl]-pyridazin-3-yl}urea;
1-(2-Fluorophenyl)-3-{6-[4-(2-trifluoromethylbenzoyl)piperazin-1-yl]pyridazin-3-yl}urea;
1-[1-(4-Fluorophenyl)ethyl]-3-{6-[4-(2-trifluoromethylbenzoyl)-piperazin-1-yl]pyridazin-3-yl}urea;
1-[1-(4-Fluorophenyl)ethyl]-3-{6-[4-(2-trifluoromethylbenzoyl)piperazin-1-yl]pyridazin-3-yl}urea;
1-[3-(4-Fluorophenyl)propyl]-3-{6-[4-(2-trifluoromethylbenzoyl)piperazin-1-yl]pyridazin-3-yl}urea;
1-Phenethyl-3-{6-[4-(2-trifluoromethylbenzoyl)piperazin-1-yl]-pyridazin-3-yl}urea;
1-(4-Fluorobenzyl)-3-{6-[4-(2-trifluoromethylbenzoyl)piperazin-1-yl]-pyridazin-3-yl}urea;
1-(3,4-Dichlorobenzyl)-3-{6-[4-(2-trifluoromethylbenzoyl)piperazin-1-yl]-pyridazin-3-yl}urea;
1-(2-Phenylcyclopropyl)-3-{6-[4-(2-trifluoromethylbenzoyl)piperazin-1-yl]-pyridazin-3-yl}urea;
1-Cyclopentyl-3-{6-[4-(2-trifluoromethylbenzoyl)piperazin-1-yl]-pyridazin-3-yl}urea;
1-(3-Cyclopropylpropyl)-3-{6-[4-(2-trifluoromethylbenzoyl)piperazin-1-yl]-pyridazin-3-yl}urea;
1-Cyclopropylmethyl-3-{6-[4-(2-trifluoromethylbenzoyl)piperazin-1-yl]-pyridazin-3-yl}urea;
1-(2-Cyclopropylethyl)-3-{6-[4-(2-trifluoromethylbenzoyl)-piperazin-1-yl]-pyridazin-3-yl}urea;
1-(2-Cyclopropylethyl)-3-{6-[4-(2-fluoro-6-trifluoromethylbenzoyl)piperazin-1-yl]-pyridazin-3-yl}urea;
1-(2-Cyclopropylethyl)-3-{6-[4-(5-fluoro-2-trifluoromethylbenzoyl)piperazin-1-yl]-pyridazin-3-yl}urea;
1-Cyclohexyl-3-{6-[4-(2-trifluoromethylbenzoy-l)piperazin-1-yl]-pyridazin-3-yl}urea;
1-(2-Cyclopropylethyl)-3-{6-[4-(2,6-difluorobenzoyl)piperazin-1-yl]-pyridazin-3-yl}urea;
1-(3-Cyclopropylpropyl)-3-{6-[4-(5-fluoro-2-trifluoromethylbenzoyl)piperazin-1-yl]-pyridazin-3-yl}urea;
4-Phenyl-N-{6-[4-(2-trifluoromethylbenzoyl)piperazin-1-yl]-pyridazin-3-yl}butyramide;
4-Methylpentanoic acid {6-[4-(2-trifluoromethylbenzoyl)-piperazin-1-yl]-pyridazin-3-yl}amide;
3-Cyclopentyl-N-{6-[4-(2-trifluoromethylbenzoyl)piperazin-1-yl]-pyridazin-3-yl}propionamide;
6-[4-(2-Trifluoromethylbenzoyl)piperazin-1-yl]-pyridazine-3-carboxylic acid phenethylamide;
6-[4-(2-Trifluoromethylbenzoyl)piperazin-1-yl]-pyridazine-3-carboxylic acid [2-(4-methoxyphenyl)ethyl]amide;
6-[4-(2-Trifluoromethylbenzoyl)piperazin-1-yl]-pyridazine-3-carboxylic acid [2-(3-fluorophenyl)ethyl]amide;
6-[4-(2-Trifluoromethylbenzoyl)piperazin-1-yl]-pyridazine-3-carboxylic acid (3-phenylpropyl)amide;
6-[4-(2-Trifluoromethylbenzoyl)piperazin-1-yl]-pyridazine-3-carboxylic acid [2-(4-fluorophenyl)ethyl]amide;
4-Methyl-2-({6-[4-(2-trifluoromethylbenzoyl)piperazin-1-yl]-pyridazine-3-carbonyl}amino)pentanoic acid methyl ester;
6-[4-(2-Trifluoromethylbenzoyl)piperazin-1-yl]-pyridazine-3-carboxylic acid (3-methylbutyl)amide;
6-[4-(5-Fluoro-2-trifluoromethylbenzoyl)piperazin-1-yl]-pyridazine-3-carboxylic acid (3-methylbutyl)amide;
6-[4-(4-Fluoro-2-trifluoromethylbenzoyl)piperazin-1-yl]-pyridazine-3-carboxylic acid (3-methylbutyl)amide;
4-Methyl-2-({6-[4-(2-trifluoromethylbenzoyl)piperazin-1-yl]-pyridazine-3-carbonyl}amino)pentanoic acid;
6-[4-(2-Trifluoromethylbenzoyl)piperazin-1-yl]-pyridazine-3-carboxylic acid (2-cyclopropylethyl)amide;
6-[4-(5-Fluoro-2-trifluoromethylbenzoyl)piperazin-1-yl]-pyridazine-3-carboxylic acid (2-cyclopropylethyl)amide;
6-[4-(2-Trifluoromethylbenzoyl)piperazin-1-yl]-pyridazine-3-carboxylic acid cyclopropylmethylamide;
4-(4-Methoxyphenyl)-N-{6-[4-(2-trifluoromethylbenzoyl)-piperazin-1-yl]-pyridazin-3-yl}butyramide;
3-(4-Fluorophenyl)-N-{6-[4-(2-trifluoromethylbenzoyl)-piperazin-1-yl]-pyridazin-3-yl}propionamide;
4-Cyclohexyl-N-{6-[4-(2-trifluoromethylbenzoyl)piperazin-1-yl]-pyridazin-3-yl}butyramide;
2,2,3,3-Tetramethylcyclopropanecarboxylic acid {6-[4-(2-trifluoromethylbenzoyl)piperazin-1-yl]pyridazin-3-yl}amide;
Cyclopropanecarboxylic acid {6-[4-(2-trifluoromethylbenzoyl)piperazin-1-yl]-pyridazin-3-yl}amide;
1-Trifluoromethylcyclopropanecarboxylic acid {6-[4-(2-trifluoromethylbenzoyl)piperazin-1-yl]-pyridazin-3-yl}amide;
2-Phenylcyclopropanecarboxylic acid {6-[4-(2-trifluoromethylbenzoyl)piperazin-1-yl]-pyridazin-3-yl}amide;
6-[4-(2-Trifluoromethylbenzoyl)piperazin-1-yl]-pyridazine-3-carboxylic acid indan-1-ylamide;
6-[4-(2-Trifluoromethylbenzoyl)piperazin-1-yl]-pyridazine-3-carboxylic acid (2-oxo-1,3-diaza-bicyclo[3.1.0]hex-3-en-4-yl)amide;
6-[4-(2-Trifluoromethylbenzoyl)piperazin-1-yl]-pyridazine-3-carboxylic acid (5-oxo-4,5-dihydro-1H-pyrazol-3-yl)amide;
6-[4-(2-Trifluoromethylbenzoyl)piperazin-1-yl]-pyridazine-3-carboxylic acid indan-5-ylamide;
5-[1,2]Dithiolan-3-yl-pentanoic acid {6-[4-(2-trifluoromethyl-benzoyl)-piperazin-1-yl]-pyridazin-3-yl}amide;
6-[4-(2-Trifluoromethyl-benzoyl)-piperazin-1-yl]-pyridazine-3-carboxylic acid (2-thiophen-2-yl-ethyl)amide;
6-[4-(2-Trifluoromethylbenzoyl)piperazin-1-yl]-pyridazine-3-carboxylic acid (2-benzo[1,3]dioxol-5-yl-ethyl)amide;
6-[4-(2-Trifluoromethylbenzoyl)piperazin-1-yl]-pyridazine-3-carboxylic acid (2,2-difluoro-2-pyridin-2-ylethyl)amide;
6-[4-(2-Trifluoromethylbenzoyl)piperazin-1-yl]-pyridazine-3-carboxylic acid (2-pyridin-2-ylethyl)amide;
6-[4-(2-Trifluoromethylbenzoyl)piperazin-1-yl]-pyridazine-3-carboxylic acid (pyridin-2-yl-methyl)amide;
6-[4-(2-Trifluoromethylbenzoyl)piperazin-1-yl]-pyridazine-3-carboxylic acid (5-chloropyridin-2-yl)amide;
6-[4-(2-Trifluoromethylbenzoyl)piperazin-1-yl]-pyridazine-3-carboxylic acid (5-trifluoromethylpyridin-2-yl)-amide;
6-[4-(2-Trifluoromethylbenzoyl)piperazin-1-yl]-pyridazine-3-carboxylic acid (7H-purin-6-yl)amide;
6-[4-(2-Trifluoromethylbenzoyl)piperazin-1-yl]-pyridazine-3-carboxylic acid pyrazin-2-ylamide;
6-[4-(2-Trifluoromethylbenzoyl)piperazin-1-yl]-pyridazine-3-carboxylic acid (1H-tetrazol-5-yl)amide;
6-[4-(2-Trifluoromethylbenzoyl)piperazin-1-yl]-pyridazine-3-carboxylic acid (2H-[1,2,4]triazol-3-yl)amide;
6-[4-(2-Trifluoromethylbenzoyl)piperazin-1-yl]-pyridazine-3-carboxylic acid (3-methylisoxazol-5-yl)amide;
6-[4-(2-Trifluoromethylbenzoyl)piperazin-1-yl]-pyridazine-3-carboxylic acid (5-methylisoxazol-3-yl)amide;
6-[4-(2-Trifluoromethylbenzoyl)piperazin-1-yl]-pyridazine-3-carboxylic acid (1H-pyrazol-3-yl)amide;
6-[4-(2-Trifluoromethylbenzoyl)piperazin-1-yl]-pyridazine-3-carboxylic acid (5-methyl-1H-pyrazol-3-yl)amide;
6-[4-(2-Trifluoromethylbenzoyl)piperazin-1-yl]-pyridazine-3-carboxylic acid pyrimidin-2-ylamide;
6-[4-(2-Trifluoromethylbenzoyl)piperazin-1-yl]-pyridazine-3-carboxylic acid pyrazin-2-ylamide;
6-[4-(2-Trifluoromethylbenzoyl)piperazin-1-yl]-pyridazine-3-carboxylic acid (4-methylpyrimidin-2-yl)amide;
6-[4-(2-Trifluoromethylbenzoyl)piperazin-1-yl]-pyridazine-3-carboxylic acid (2-oxo-2,3-dihydro-pyrimidin-4-yl)amide;
6-[4-(2-Trifluoromethylbenzoyl)piperazin-1-yl]-pyridazine-3-carboxylic acid (6-oxo-1,6-dihydro-pyrimidin-2-yl)amide;
6-[4-(2-Trifluoromethylbenzoyl)piperazin-1-yl]-pyridazine-3-carboxylic acid [1,3,4]thiadiazol-2-ylamide;
6-[4-(2-Trifluoromethylbenzoyl)piperazin-1-yl]-pyridazine-3-carboxylic acid thiazol-2-ylamide;
6-[4-(2-Trifluoromethylbenzoyl)piperazin-1-yl]-pyridazine-3-carboxylic acid pyridin-2-ylamide;
6-[4-(2-Trifluoromethylbenzoyl)piperazin-1-yl]-pyridazine-3-carboxylic acid pyridazin-3-ylamide;
6-[4-(2-Trifluoromethylbenzoyl)piperazin-1-yl]-pyridazine-3-carboxylic acid pyridin-3-ylamide;
6-[4-(2-Trifluoromethylbenzoyl)piperazin-1-yl]-pyridazine-3-carboxylic acid pyridin-4-ylamide;
6-[4-(2-Trifluoromethylbenzoyl)piperazin-1-yl]-pyridazine-3-carboxylic acid (6-oxo-1,6-dihydro-[1,3,5]triazin-2-yl)amide;
6-[4-(2-Trifluoromethylbenzoyl)piperazin-1-yl]-pyridazine-3-carboxylic acid (5-fluoropyridin-2-yl)amide;
6-[4-(2-Trifluoromethylbenzoyl)-piperazin-1-yl]-pyridazine-3-carboxylic acid (5-cyanopyridin-2-yl)amide;
6-[4-(2-Trifluoromethylbenzoyl)-piperazin-1-yl]-pyridazine-3-carboxylic acid (4,6-dimethyl-pyrimidin-2-yl)amide;
6-[4-(2-Trifluoromethylbenzoyl)-piperazin-1-yl]-pyridazine-3-carboxylic acid (2-chloropyridin-4-yl)amide;
6-[4-(2-Trifluoromethylbenzoyl)-piperazin-1-yl]-pyridazine-3-carboxylic acid (1H-indol-6-yl)amide;
6-[4-(2-Trifluoromethylbenzoyl)-piperazin-1-yl]-pyridazine-3-carboxylic acid (1H-indol-4-yl)amide;
6-[4-(2-Trifluoromethylbenzoyl)-piperazin-1-yl]-pyridazine-3-carboxylic acid (1H-indazol-5-yl)amide;
6-[4-(2-Trifluoromethylbenzoyl)-piperazin-1-yl]-pyridazine-3-carboxylic acid (1H-indazol-6-yl)amide;
6-[4-(2-Trifluoromethylbenzoyl)-piperazin-1-yl]-pyridazine-3-carboxylic acid (4-methylthiazol-2-yl)amide;
6-[4-(2-Trifluoromethylbenzoyl)-piperazin-1-yl]-pyridazine-3-carboxylic acid (5-methylthiazol-2-yl)amide;
6-[4-(2-Trifluoromethylbenzoyl)-piperazin-1-yl]-pyridazine-3-carboxylic acid (5-thioxo-4,5-dihydro-1H-[1,2,4]triazol-3-yl)amide;
6-[4-(2-Trifluoromethylbenzoyl)piperazin-1-yl]-pyridazine-3-carboxylic acid (1H-benzoimidazol-2-yl)amide;
6-[4-(2-Trifluoromethylbenzoyl)piperazin-1-yl]-pyridazine-3-carboxylic acid (6-methylpyridazin-3-yl)amide;
6-[4-(2-Trifluoromethylbenzoyl)piperazin-1-yl]-pyridazine-3-carboxylic acid (6-methoxypyridazin-3-yl)amide;
6-[4-(2-Trifluoromethylbenzoyl)-piperazin-1-yl]-pyridazine-3-carboxylic acid (6-chloropyridazin-3-yl)amide;
6-[4-(2-Trifluoromethylbenzoyl)piperazin-1-yl]-pyridazine-3-carboxylic acid (4-chlorophenyl)amide;
6-[4-(2-Trifluoromethylbenzoyl)piperazin-1-yl]-pyridazine-3-carboxylic acid (4-carbamoyl-phenyl)amide;
6-[4-(2-Trifluoromethylbenzoyl)piperazin-1-yl]-pyridazine-3-carboxylic acid (3-carbamoyl-phenyl)amide;
6-[4-(2-Trifluoromethylbenzoyl)piperazin-1-yl]-pyridazine-3-carboxylic acid m-tolylamide;
6-[4-(2-Trifluoromethylbenzoyl)piperazin-1-yl]-pyridazine-3-carboxylic acid p-tolylamide;
6-[4-(2-Trifluoromethylbenzoyl)piperazin-1-yl]-pyridazine-3-carboxylic acid o-tolylamide;
6-[4-(2-Trifluoromethylbenzoyl)piperazin-1-yl]-pyridazine-3-carboxylic acid (2-propylphenyl)amide;
6-[4-(2-Trifluoromethylbenzoyl)piperazin-1-yl]-pyridazine-3-carboxylic acid (4-propylphenyl)amide;
6-[4-(2-Trifluoromethylbenzoyl)piperazin-1-yl]-pyridazine-3-carboxylic acid (4-isopropylphenyl)amide;
6-[4-(2-Trifluoromethylbenzoyl)piperazin-1-yl]-pyridazine-3-carboxylic acid (2-isopropylphenyl)amide;
6-[4-(2-Trifluoromethylbenzoyl)piperazin-1-yl]-pyridazine-3-carboxylic acid (2-chlorophenyl)amide;
6-[4-(2-Trifluoromethylbenzoyl)piperazin-1-yl]-pyridazine-3-carboxylic acid (2-cyano-3-fluorophenyl)amide;
6-[4-(2-Trifluoromethylbenzoyl)piperazin-1-yl]-pyridazine-3-carboxylic acid (2,4-dimethylphenyl)amide;
6-[4-(2-Trifluoromethylbenzoyl)piperazin-1-yl]-pyridazine-3-carboxylic acid (2,5-dimethylphenyl)amide;
6-[4-(2-Trifluoromethylbenzoyl)piperazin-1-yl]-pyridazine-3-carboxylic acid (2,6-dimethylphenyl)amide;
6-[4-(2-Trifluoromethylbenzoyl)piperazin-1-yl]-pyridazine-3-carboxylic acid (2,3-dimethylphenyl)amide;
6-[4-(2-Trifluoromethylbenzoyl)piperazin-1-yl]-pyridazine-3-carboxylic acid (3,5-dimethylphenyl)amide;
6-[4-(2-Trifluoromethylbenzoyl)piperazin-1-yl]-pyridazine-3-carboxylic acid (3,4-dimethylphenyl)amide;
6-[4-(2-Trifluoromethylbenzoyl)piperazin-1-yl]-pyridazine-3-carboxylic acid (4-ethylphenyl)amide;
6-[4-(2-Trifluoromethylbenzoyl)piperazin-1-yl]-pyridazine-3-carboxylic acid (2-ethylphenyl)amide;
6-[4-(2-Trifluoromethylbenzoyl)piperazin-1-yl]-pyridazine-3-carboxylic acid (3-fluoro-2-methylphenyl)amide;
6-[4-(2-Trifluoromethylbenzoyl)piperazin-1-yl]-pyridazine-3-carboxylic acid (2-fluoro-4-methylphenyl)amide;
6-[4-(2-Trifluoromethylbenzoyl)piperazin-1-yl]-pyridazine-3-carboxylic acid (4-fluoro-2-methylphenyl)amide;
6-[4-(2-Trifluoromethylbenzoyl)piperazin-1-yl]-pyridazine-3-carboxylic acid (2-fluoro-5-methylphenyl)amide;
6-[4-(2-Trifluoromethylbenzoyl)piperazin-1-yl]-pyridazine-3-carboxylic acid (3-fluoro-5-methylphenyl)amide;
6-[4-(2-Trifluoromethylbenzoyl)piperazin-1-yl]-pyridazine-3-carboxylic acid (3-fluorophenyl)amide;
6-[4-(2-Trifluoromethylbenzoyl)piperazin-1-yl]-pyridazine-3-carboxylic acid (2-fluorophenyl)amide;
6-[4-(2-Trifluoromethylbenzoyl)piperazin-1-yl]-pyridazine-3-carboxylic acid (4-fluorophenyl)amide;
6-[4-(2-Trifluoromethylbenzoyl)piperazin-1-yl-pyridazine-3-carboxylic acid (2,4-difluorophenyl)amide;
6-[4-(2-Trifluoromethylbenzoyl)piperazin-1-yl]-pyridazine-3-carboxylic acid (2,5-difluorophenyl)amide;
6-[4-(2-Trifluoromethylbenzoyl)piperazin-1-yl]-pyridazine-3-carboxylic acid (3,4-difluorophenyl)amide;
6-[4-(2-Trifluoromethylbenzoyl)piperazin-1-yl]-pyridazine-3-carboxylic acid (2,3-difluorophenyl)amide;
6-[4-(2-Trifluoromethylbenzoyl)piperazin-1-yl]-pyridazine-3-carboxylic acid (2,6-difluorophenyl)amide;
6-[4-(2-Trifluoromethylbenzoyl)piperazin-1-yl]-pyridazine-3-carboxylic acid (4-cyanophenyl)amide;
6-[4-(2-Trifluoromethylbenzoyl)piperazin-1-yl]-pyridazine-3-carboxylic acid (2-cyanophenyl)amide;
6-[4-(2-Trifluoromethylbenzoyl)piperazin-1-yl]-pyridazine-3-carboxylic acid (3-cyanophenyl)amide;
6-[4-(2-Trifluoromethylbenzoyl)piperazin-1-yl]-pyridazine-3-carboxylic acid (3-chlorophenyl)amide;
6-[4-(2-Trifluoromethylbenzoyl)piperazin-1-yl]-pyridazine-3-carboxylic acid (3-chloro-2-methylphenyl)amide;
6-[4-(2-Trifluoromethylbenzoyl)piperazin-1-yl]-pyridazine-3-carboxylic acid (2-chloro-3-methylphenyl)amide;
6-[4-(2-Trifluoromethylbenzoyl)piperazin-1-yl]-pyridazine-3-carboxylic acid (2,5-dichlorophenyl)amide;
6-[4-(2-Trifluoromethylbenzoyl)piperazin-1-yl]-pyridazine-3-carboxylic acid (2-chloro-5-methylphenyl)amide;
6-[4-(2-Trifluoromethylbenzoyl)piperazin-1-yl]-pyridazine-3-carboxylic acid (2-chloro-6-methylphenyl)amide;
6-[4-(2-Trifluoromethylbenzoyl)piperazin-1-yl]-pyridazine-3-carboxylic acid (4-chloro-2-methylphenyl)amide;
6-[4-(2-Trifluoromethylbenzoyl)piperazin-1-yl]-pyridazine-3-carboxylic acid (4-chloro-3-methylphenyl)amide;
6-[4-(2-Trifluoromethylbenzoyl)piperazin-1-yl]-pyridazine-3-carboxylic acid (3-chloro-4-methylphenyl)amide;
6-[4-(2-Trifluoromethylbenzoyl)piperazin-1-yl]-pyridazine-3-carboxylic acid (2-chloro-4-methylphenyl)amide;
6-[4-(2-Trifluoromethylbenzoyl)piperazin-1-yl]-pyridazine-3-carboxylic acid (2-chloro-5-fluorophenyl)amide;
6-[4-(2-Trifluoromethylbenzoyl)piperazin-1-yl]-pyridazine-3-carboxylic acid (5-chloro-2-fluorophenyl)amide;
6-[4-(2-Trifluoromethylbenzoyl)piperazin-1-yl]-pyridazine-3-carboxylic acid (2,5-difluorophenyl)amide;
6-[4-(2-Trifluoromethylbenzoyl)piperazin-1-yl]-pyridazine-3-carboxylic acid (2,6-dichlorophenyl)amide;
6-[4-(2-Trifluoromethylbenzoyl)piperazin-1-yl]-pyridazine-3-carboxylic acid (2-trifluoromethylphenyl)amide;
6-[4-(2-Trifluoromethylbenzoyl)piperazin-1-yl]-pyridazine-3-carboxylic acid (4-trifluoromethylphenyl)amide;
6-[4-(2-Trifluoromethylbenzoyl)-piperazin-1-yl]-pyridazine-3-carboxylic acid (3-trifluoromethylphenyl)amide;
6-[4-(2-Trifluoromethylbenzoyl)piperazin-1-yl]-pyridazine-3-carboxylic acid phenylamide;
6-[4-(2-Trifluoromethylbenzoyl)piperazin-1-yl]-pyridazine-3-carboxylic acid (5-chloro-2-methoxyphenyl)amide;
6-[4-(2-Trifluoromethylbenzoyl)piperazin-1-yl]-pyridazine-3-carboxylic acid (2,5-dimethoxyphenyl)amide;
6-[4-(2-Trifluoromethylbenzoyl)piperazin-1-yl]-pyridazine-3-carboxylic acid (2-chloro-4-methoxyphenyl)amide;
6-[4-(2-Trifluoromethylbenzoyl)-piperazin-1-yl]-pyridazine-3-carboxylic acid (4-methoxyphenyl)amide;
6-[4-(2-Trifluoromethylbenzoyl)piperazin-1-yl]-pyridazine-3-carboxylic acid (2-methoxyphenyl)amide;
6-[4-(2-Trifluoromethylbenzoyl)piperazin-1-yl]-pyridazine-3-carboxylic acid (3-methoxyphenyl)amide;
4-({6-[4-(2-Trifluoromethylbenzoyl)piperazin-1-yl]-pyridazine-3-carbonyl}amino)-benzoic acid methyl ester;
4-({6-[4-(2-Trifluoromethylbenzoyl)piperazin-1-yl]-pyridazine-3-carbonyl}amino)-benzoic acid;
2-({6-[4-(2-Trifluoromethylbenzoyl)piperazin-1-yl]-pyridazine-3-carbonyl}amino)-benzoic acid methyl ester;
2-({6-[4-(2-Trifluoromethylbenzoyl)piperazin-1-yl]-pyridazine-3-carbonyl}amino)-benzoic acid;
6-[4-(2-Trifluoromethylbenzoyl)piperazin-1-yl]-pyridazine-3-carboxylic acid (3,4-dichlorophenyl)amide;
6-[4-(2-Trifluoromethylbenzoyl)piperazin-1-yl]-pyridazine-3-carboxylic acid [2-(2,4-fluorophenyl)ethyl]amide;
6-[4-(2-Trifluoromethylbenzoyl)piperazin-1-yl]-pyridazine-3-carboxylic acid [2-(2-fluorophenyl)ethyl]amide;
6-[4-(2-Trifluoromethylbenzoyl)piperazin-1-yl]-pyridazine-3-carboxyli-c acid [2-(4-chlorophenyl)ethyl]amide;
6-[4-(2-Trifluoromethylbenzoyl)piperazin-1-yl]-pyridazine-3-carboxylic acid [2-(3-chlorophenyl)ethyl]amide;
6-[4-(2-Trifluoromethylbenzoyl)piperazin-1-yl]-pyridazine-3-carboxylic acid (2-phenylpropyl)amide;
6-[4-(2-Trifluoromethylbenzoyl)piperazin-1-yl]-pyridazine-3-carboxylic acid (2-biphenyl-4-ylethyl)amide;
(R)-6-[4-(2-Trifluoromethylbenzoyl)piperazin-1-yl]-pyridazine-3-carboxylic acid (2-hydroxy-2-phenylethyl)amide;
(S)-6-[4-(2-Trifluoromethylbenzoyl)-piperazin-1-yl]-pyridazine-3-carboxylic acid (2-hydroxy-2-phenylethyl)amide;
Acetic acid 1-phenyl-2-({6-[4-(2-trifluoromethyl-benzoyl)-piperazin-1-yl]-pyridazine-3-carbonyl}amino)ethyl ester;
6-[4-(2-Trifluoromethylbenzoyl)piperazin-1-yl]-pyridazine-3-carboxylic acid [3-(4-fluorophenyl)propyl]amide;
6-[4-(2-Trifluoromethylbenzoyl)piperazin-1-yl]-pyridazine-3-carboxylic acid (2,2-difluoro-2-phenylethyl)amide;
6-[4-(2-Trifluoromethylbenzoyl)piperazin-1-yl]-pyridazine-3-carboxylic acid [2-(3-fluorophenyl)-2-hydroxyethyl]amide;
6-[4-(2-Trifluoromethylbenzoyl)piperazin-1-yl]-pyridazine-3-carboxylic acid (4-hydroxybutyl)amide;
6-[4-(2-Trifluoromethylbenzoyl)piperazin-1-yl]-pyridazine-3-carboxylic acid (3-hydroxy-4,4-dimethylpentyl)amide;
6-[4-(2-Trifluoromethylbenzoyl)piperazin-1-yl]-pyridazine-3-carboxylic acid (3-hydroxy-3-methylbutyl)amide;
6-[4-(2-Trifluoromethylbenzoyl)piperazin-1-yl]-pyridazine-3-carboxylic acid (2-hydroxy-3,3-dimethylbutyl)amide;
6-[4-(5-Fluoro-2-trifluoromethylbenzoyl)piperazin-1-yl]-pyridazine-3-carboxylic acid (2-hydroxy-3,3-dimethylbutyl)amide;
6-[4-(2-Nitrobenzoyl)piperazin-1-yl]-pyridazine-3-carboxylic acid (3-methylbutyl)amide;
6-[4-(2-Chlorobenzoyl)piperazin-1-yl]-pyridazine-3-carboxylic acid (3-methylbutyl)amide;
6-[4-(2,4-Dichlorobenzoyl)piperazin-1-yl]-pyridazine-3-carboxylic acid (3-methylbutyl)amide;
6-[4-(2-Aminobenzoyl)piperazin-1-yl]-pyridazine-3-carboxylic acid (3-methylbutyl)amide;
6-[4-(2-Trifluoromethylbenzoyl)piperazin-1-yl]-pyridazine-3-carboxylic acid [2-(4-chlorophenoxy)ethyl]amide;
6-[4-(2-Trifluoromethylbenzoyl)piperazin-1-yl]-pyridazine-3-carboxylic acid [2-(4-fluorophenoxy)ethyl]amide;
6-[4-(2-Trifluoromethylbenzoyl)piperazin-1-yl]-pyridazine-3-carboxylic acid (3,3-dimethylbutyl)amide;
6-[4-(2-Trifluoromethylbenzoyl)piperazin-1-yl]-pyridazine-3-carboxylic acid pentylamide;
4-({6-[4-(2-Trifluoromethylbenzoyl)piperazin-1-yl]-pyridazine-3-carbonyl}amino)butyric acid ethyl ester;
6-[4-(5-Fluoro-2-trifluoromethylbenzoyl)piperazin-1-yl]-pyridazine-3-carboxylic acid pentylamide;
6-[4-(2-Trifluoromethylbenzoyl)piperazin-1-yl]-pyridazine-3-carboxylic acid (4-methylpentyl)amide;
6-[4-(2-Fluoro-6-trifluoromethylbenzoyl)piperazin-1-yl]-pyridazine-3-carboxylic acid (3-methylbutyl)amide;
6-[4-(2,6-Difluorobenzoyl)piperazin-1-yl]-pyridazine-3-carboxylic acid (3-methylbutyl)amide;
6-[4-(2-Trifluoromethylbenzoyl)piperazin-1-yl]-pyridazine-3-carboxylic acid (2-oxo-2-phenylethyl)amide;
Acetic acid 1,1-dimethyl-3-({6-[4-(2-trifluoromethyl-benzoyl)piperazin-1-yl]-pyridazine-3-carbonyl}amino)propyl ester;
6-[4-(2-Trifluoromethylbenzoyl)piperazin-1-yl]-pyridazine-3-carboxylic acid (2-phenoxyethyl)amide;
6-[4-(2-Trifluoromethylbenzoyl)piperazin-1-yl]-pyridazine-3-carboxylic acid hexylamide;
6-[4-(2-Trifluoromethylbenzoyl)piperazin-1-yl]-pyridazine-3-carboxylic acid (4-methylpentyl)amide;
6-[4-(5-Fluoro-2-trifluoromethylbenzoyl)piperazin-1-yl]-pyridazine-3-carboxylic acid (4-methylpentyl)amide;
6-[2,5-Dimethyl-4-(2-trifluoromethylbenzoyl)piperazin-1-yl]-pyridazine-3-carboxylic acid pentylamide;
6-[4-(2-Trifluoromethylbenzoyl)-piperazin-1-yl]-pyridazine-3-carboxylic acid heptylamide;
6-[4-(2-Sulfamoylbenzoyl)-piperazin-1-yl]-pyridazine-3-carboxylic acid (3-methylbutyl)-amide;
6-[4-(5-Chloro-2-trifluoromethyl-benzoyl)-piperazin-1-yl]-pyridazine-3-carboxylic acid hexylamide;
6-[4-(2-Trifluoromethylbenzoyl)-piperazin-1-yl]-pyridazine-3-carboxylic acid (2-cyclopropyl-2-oxoethyl)amide;
4-Trifluoromethyl-6-[4-(2-trifluoromethylbenzoyl)-piperazin-1-yl]-pyridazine-3-carboxylic acid (3-methyl-butyl)amide;
6-[4-(5-Fluoro-2-trifluoromethylbenzoyl)piperazin-1-yl]-pyridazine-3-carboxylic acid pentyl-4-enylamide;
6-(4-Benzoylpiperazin-1-yl)-pyridazine-3-carboxylic acid (2-cyclopropylethyl)amide;
6-[4-(2-Chloro-5-fluorobenzoyl)piperazin-1-yl]-pyridazine-3-carboxylic acid (2-cyclopropylethyl)amide;
6-[4-(5-Chloro-2-trifluoromethylbenzoyl)piperazin-1-yl]-pyridazine-3-carboxylic acid (2-cyclopropylethyl)amide;
6-[4-(2,6-Difluorobenzoyl)piperazin-1-yl]-pyridazine-3-carboxylic acid (2-cyclopropylethyl)amide;
6-[4-(2,5-Bis-trifluoromethylbenzoyl)piperazin-1-yl]-pyridazine-3-carboxylic acid (2-cyclopropylethyl)amide;
6-[4-(2,4-Bis-trifluoromethylbenzoyl)piperazin-1-yl]-pyridazine-3-carboxylic acid (2-cyclopropylethyl)amide;
6-[4-(2,5-Difluorobenzoyl)piperazin-1-yl]-pyridazine-3-carboxylic acid (2-cyclopropylethyl)amide;
6-[4-(5-Fluoro-2-trifluoromethylbenzoyl)piperazin-1-yl]-pyridazine-3-carboxylic acid (3-cyclopropylpropyl)amide;
6-[4-(2-Fluorobenzoyl)piperazin-1-yl]-pyridazine-3-carboxylic acid (2-cyclopropylethyl)amide;
6-[4-(3-Fluoro-2-trifluoromethylbenzoyl)piperazin-1-yl]-pyridazine-3-carboxylic acid (2-cyclopropylethyl)amide;
6-[4-(4-Fluoro-2-trifluoromethylbenzoyl)piperazin-1-yl]-pyridazine-3-carboxylic acid (3-cyclopropylpropyl)amide;
6-[4-(2-Trifluoromethylbenzoyl)piperazin-1-yl]-pyridazine-3-carboxylic acid (2-methylcyclopropylmethyl)amide;
6-[4-(5-Fluoro-2-methoxybenzoyl)piperazin-1-yl]-pyridazine-3-carboxylic acid (2-cyclopropylethyl)amide;
6-[4-(2-Dimethylaminobenzoyl)piperazin-1-yl]-pyridazine-3-carboxylic acid (2-cyclopropylethyl)amide;
6-[4-(2-Chloro-5-dimethylaminobenzoyl)piperazin-1-yl]-pyridazine-3-carboxylic acid (2-cyclopropylethyl)amide;
6-[4-(2,5-Dimethylbenzoyl)piperazin-1-yl]-pyridazine-3-carboxylic acid (2-cyclopropylethyl)amide;
6-[4-(2,5-Dichlorobenzoyl)piperazin-1-yl]-pyridazine-3-carboxylic acid (2-cyclopropylethyl)amide;
6-[4-(2-Trifluoromethylbenzoyl)piperazin-1-yl]-pyridazine-3-carboxylic acid cyclobutylmethylamide;
Acetic acid 2-{4-[6-(2-cyclopropylethylcarbamoyl)-pyridazin-3-yl]-piperazine-1-carbonyl}phenyl ester;
6-[4-(2-Trifluoromethylbenzoyl)piperazin-1-yl]-pyridazine-3-carboxylic acid (2-cyclopropyl-2-hydroxyethyl)amide;
6-[4-(2-Trifluoromethylbenzoyl)piperazin-1-yl]-pyridazine-3-carboxylic acid (2-phenylcyclopropylmethyl)amide;
6-[4-(2-Trifluoromethylbenzoyl)piperazin-1-yl]-pyridazine-3-carboxylic acid (3-cyclopropylpropyl)amide;
6-[4-(2-Cyanobenzoyl)piperazin-1-yl]-pyridazine-3-carboxylic acid (2-cyclopropylethyl)amide;
6-{4-[2-(2-Trifluoromethylphenyl)acetyl]-piperazin-1-yl}pyridazine-3-carboxylic acid (2-cyclopropylethyl)amide;
6-[4-(4-Fluoro-2-trifluoromethylbenzoyl)piperazin-1-yl]-pyridazine-3-carboxylic acid (2-cyclopropylethyl)amide;
6-[4-(5-Chloro-2-trifluoromethylbenzoyl)piperazin-1-yl]-pyridazine-3-carboxylic acid (3-cyclopropylpropyl)amide;
6-[3,5-Dimethyl-4-(2-trifluoromethylbenzoyl)piperazin-1-yl]-pyridazine-3-carboxylic acid (2-cyclopropylethyl)amide;
2-{4-[6-(2-Cyclopropylethylcarbamoyl)pyridazin-3-yl]-piperazine-1-carbonyl}benzoic acid methyl ester;
6-[4-(2-Trifluoromethyl-benzoyl)-piperazin-1-yl]-pyridazine-3-carboxylic acid (2-cyclobutylethyl)amide;
2-{4-[6-(2-Cyclopropyl-ethylcarbamoyl)-pyridazin-3-yl]-piperazine-1-carbonyl}-benzoic acid;
6-[4-(5-Chloro-2-trifluoromethyl-benzoyl)-piperazin-1-yl]-pyridazine-3-carboxylic acid (2-cyclobutylethyl)amide;
6-[4-(5-Fluoro-2-trifluoromethylbenzoyl)-piperazin-1-yl]-pyridazine-3-carboxylic acid (2-cyclobutylethyl)amide;
6-[4-(2-Trifluoromethylbenzoyl)-piperazin-1-yl]-pyridazine-3-carboxylic acid (3-cyclobutyl-propyl)amide;
6-[4-(5-Fluoro-2-trifluoromethylbenzoyl)-piperazin-1-yl]-pyridazine-3-carboxylic acid (2-cyclopropylethyl)amide;
6-[4-(2-Trifluoromethyl-thiobenzoyl)-piperazin-1-yl]-pyridazine-3-carboxylic acid (2-cyclopropylethyl)amide;
6-[4-(2-Trifluoromethylbenzoyl)-piperazin-1-yl]-pyridazine-3-carboxylic acid (4-cyclopropylbutyl)amide;
6-[4-(2-Trifluoromethylbenzoyl)-piperazin-1-yl]-pyridazine-3-carboxylic acid (2,2-dimethyl-cyclopropylmethyl)amide;
6-[4-(Pyridine-2-carbonyl)piperazin-1-yl]-pyridazine-3-carboxylic acid (2-cyclopropylethyl)amide;
6-[4-(2-trifluoromethylfuran-3-carbonyl)piperazin-1-yl]-pyridazine-3-carboxylic acid (2-cyclopropylethyl)amide;
6-[4-(2-Chloro-4-trifluoromethylpyrimidine-5-carbonyl)piperazin-1-yl]-pyridazine-3-carboxylic acid (2-cyclopropylethyl)amide;
6-[4-(5-Methyl-2-trifluoromethylfuran-3-carbonyl)piperazin-1-yl]-pyridazine-3-carboxylic acid (2-cyclopropylethyl)amide;
6-[4-(2-Chloropyridine-3-carbonyl)piperazin-1-yl]-pyridazine-3-carboxylic acid (2-cyclopropylethyl)amide;
6-[4-(2-Methyl-5-trifluoromethyloxazole-4-carbonyl)piperazin-1-yl]-pyridazine-3-carboxylic acid (2-cyclopropylethyl)amide;
6-[4-(2,6-Dichloropyridine-3-carbonyl)piperazin-1-yl]-pyridazine-3-carboxylic acid (2-cyclopropylethyl)amide;
6-[4-(Pyrrolidine-1-carbonyl)piperazin-1-yl]-pyridazine-3-carboxylic acid (2-cyclopropylethyl)amide;
6-[4-(1-Methyl-1H-pyrrole-2-carbonyl)piperazin-1-yl]-pyridazine-3-carboxylic acid (2-cyclopropylethyl)amide;
6-[4-(Tetrahydrofuran-2-carbonyl)piperazin-1-yl]-pyridazine-3-carboxylic acid (2-cyclopropylethyl)amide;
6-{4-[2-(2-Trifluoromethylphenyl)acetyl]piperazin-1-yl}-pyridazine-3-carboxylic acid (2-cyclopropylethyl)amide;
4-[6-(3-Methylbutylcarbamoyl)pyridazin-3-yl]-piperazine-1-carboxylic acid t-butyl ester;
4-[6-(2-Cyclopropylethylcarbamoyl)pyridazin-3-yl]-piperazine-1-carboxylic acid t-butyl ester;
6-[4-(4,4,4-Trifluoro-3-hydroxy-3-trifluoromethylbutyryl)piperazin-1-yl]-pyridazine-3-carboxylic acid (2-cyclopropylethyl)amide;
6-[4-(4,4,4-Trifluoro-3-hydroxy-3-methylbutyryl)piperazin-1-yl]-pyridazine-3-carboxylic acid (2-cyclopropylethyl)amide;
and 6-[4-(3,3,3-Trifluoro-2-hydroxy-2-methylpropionyl)piperazin-1-yl]-pyridazine-3-carboxylic acid (2-cyclopropylethyl)amide;
6-[4-(1-Hydroxycyclopropanecarbonyl)piperazin-1-yl]-pyridazine-3-carboxylic acid (2-cyclopropylethyl)amide;
6-(4-Cyclobutanecarbonylpiperazin-1-yl)pyridazine-3-carboxylic acid (2-cyclopropylethyl)amide;
6-[4-(2-Trifluoromethylcyclopropanecarbonyl)piperazin-1-yl]-pyridazine-3-carboxylic acid (2-cyclopropylethyl)amide;
6-(4-Cyclohexanecarbonylpiperazin-1-yl)pyridazine-3-carboxylic acid (2-cyclopropylethyl)amide;
6-[4-(2-Methylcyclohexanecarbonyl)piperazin-1-yl]-pyridazine-3-carboxylic acid (2-cyclopropylethyl)amide;
6-[4-(3-Methylcyclohexanecarbonyl)piperazin-1-yl]-pyridazine-3-carboxylic acid (2-cyclopropylethyl)amide;
6-[4-(4-Methylcyclohexanecarbonyl)piperazin-1-yl]-pyridazine-3-carboxylic acid (2-cyclopropylethyl)amide;
6-[4-(2-Methylcyclopropanecarbonyl)piperazin-1-yl]-pyridazine-3-carboxylic acid (2-cyclopropylethyl)amide;
6-[4-(2,2,3,3-Tetramethylcyclopropanecarbonyl)piperazin-1-yl]pyridazine-3-carboxylic acid (2-cyclopropylethyl)amide;
6-[4-(2-Ethylbutyryl)piperazin-1-yl]-pyridazine-3-carboxylic acid (2-cyclopropylethyl)amide;
6-[4-(3,3,3-Trifluoro-2-methyl-2-trifluoromethylpropionyl)piperazin-1-yl]-pyridazine-3-carboxylic acid (2-cyclopropylethyl)amide;
6-[4-(2,2-Dimethylpropionyl)piperazin-1-yl]-pyridazine-3-carboxylic acid (2-cyclopropylethyl)amide;
6-[4-(2,2-Dimethylbutyryl)piperazin-1-yl]-pyridazine-3-carboxylic acid (2-cyclopropylethyl)amide;
6-[4-(2,2-Dimethylpentanoyl)piperazin-1-yl]-pyridazine-3-carboxylic acid (2-cyclopropylethyl)amide;
6-[4-(4,4,4-Trifluorobut-2-enoyl)piperazin-1-yl]-pyridazine-3-carboxylic acid (2-cyclopropylethyl)amide; and
6-[4-(4,4,4-Trifluoro-3-trifluoromethylbut-2-enoyl)-piperazin-1-yl]-pyridazine-3-carboxylic acid (2-cyclopropyl-ethyl)amide;
   or a pharmaceutically acceptable salt thereof.

DGAT is a critical catalyst in triglyceride synthesis in the two biological pathways that use Acyl CoA to synthesize triglycerides. Two distinct gene classes of the enzyme, DGAT1 and DGAT2, have been identified based on cloning. Pharmacological effects of DGAT inhibition in mice include: increased insulin sensitivity, increased leptin sensitivity and resistance to diet induced obesity.

DGAT inhibitors for the purpose of the present invention include, but are not limited to, compounds as defined in WO 2005044250, WO 2005013907, WO 2004094618 and WO 2004047755, in each case, the claims, the final products of the working examples and methods of making the same, and the pharmaceutical preparations thereof, are incorporated herein by reference.

Protein tyrosine phosphatases (PTPase) along with protein tyrosine kinases are known to be key regulators of insulin signal transduction. Protein tyrosine phosphatase 1B (PTP1B) has been shown to inhibit insulin phosphorylation of the Insulin Receptor (IR) and insulin receptor substrates. Mice deficient in PTP-1B expression show increased insulin sensitivity and low adiposity and weight gain resistance on a high fat diet. Treatment of obese mice (ob/ob) with a PTP-1B antisense oligonucleotide (ASO, ISIS-113715) is shown to result in decrease of PTP-1B mRNA and protein expression with subsequent improvement in insulin sensitivity and normalization of glucose levels. Vanadium complexes have also been shown to inhibit many PTPases, including PTP-1B, and to enhance insulin sensitivity both in rodent models of diabetes and in diabetic patients. Two Vanadium complexes shown in formula (X), BMOV and BEOV, which are closely related analogues have surprisingly shown promise in the treatment of diabetes. Carol L. Winter et al. (Exp. Biol. & Med 2005, 230:207-216) have demonstrated that PTPase inhibition with BMOV treatment resulted in prevention of development of diabetes in ZDF rats, along with improvement in pancreatic β-cell function and architecture:

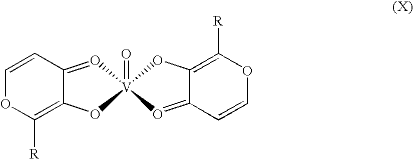

R=Me, Bis(maltolato)oxovanadium(IV), BMOV
R=Et, Bisethylmaltolato)oxovanadium(IV), BEOV

Furthermore, PTPase inhibitors for the purpose of the present invention include, but are not limited to, compounds as defined in WO 2005035551, WO 2004050646, WO 2004062664 and WO 2004041799, in each case, the claims, the final products of the working examples and methods of making the same, and the pharmaceutical preparations thereof, are incorporated herein by reference.

Preferred are the PTPase inhibitors of the formula

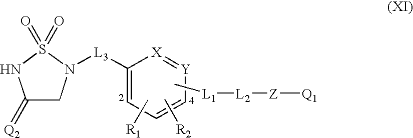

wherein
R₁ is hydrogen, halogen, hydroxy, alkoxy, carboxy, cyano, nitro, trifluoromethyl, alkynyl, alkylthio, heteroaralkyl, heteroaralkoxy or heteroaryloxy provided that R₁ is located at the 2-position when L₃ is —(CHR)ₛ— in which s is zero; or
R₁ is optionally substituted alkyl, alkenyl, optionally substituted amino, aralkyl, aralkoxy, aralkylthio, aryloxy, arylthio or cycloalkyl provided that a monocyclic aryl group which is substituted at the para position with a methylene or ethylene bridged nitrogen containing heterocycle does not constitute part of R₁ when
   (i) R₁ is located at the 2-position and L₃ is —(CHR)ₛ— in which s is zero;
   (ii) X and Y each are CH; and
   (iii) Q₂ is oxygen; or
C—R₁ may be replaced with nitrogen or N→O; or
R₁ and R₂ combined together with the carbon atoms to which R₁ and R₂ are attached form an optionally substituted fused 5- to 6-membered aromatic or heteroaromatic ring provided that R₁ and R₂ are attached to carbon atoms adjacent to each other; or
R₂ is hydrogen, halogen, hydroxy, alkoxy, cyano, trifluoromethyl, nitro, optionally substituted amino, optionally substituted alkyl, alkylthio, aralkyl, heteroaralkyl, aralkoxy, heteroaralkoxy, aralkylthio, aryloxy, heteroaryloxy, arylthio or cycloalkyl; or
R₂ is —C(O)R₃ wherein
   R₃ is hydroxy or optionally substituted alkoxy; or
   R₃ is —NR₄R₅ in which R₄ and R₅ are independently hydrogen, optionally substituted alkyl, alkenyl, alkynyl, cycloalkyl, aryl, heterocyclyl, aralkyl or heteroaralkyl;
   L₁ is a single bond; or
   L₁ is carbon which combined together with R₂ and the carbon atoms to which L₁ and R₂ are attached form an optionally substituted fused 5- or 6-membered aromatic or heteroaromatic ring provided that L₁ and R₂ are attached to carbon atoms adjacent to each other; or
   L₁ is CH or nitrogen which taken together with R₂ and the carbon atoms to which L₁ and R₂ are attached form a fused 5- to 7-membered ring which may be interrupted with one or two heteroatoms selected from oxygen, nitrogen and sulfur provided that L₁ and R₂ are attached to carbon atoms adjacent to each other; or
   L₁ is CH, oxygen, sulfur or nitrogen and L₂ is carbon which combined together with L₁, R₂ and the carbon atoms to which L₁ and R₂ are attached form an optionally substituted fused 5- or 6-membered aromatic or heteroaromatic ring provided that L₁ and R₂ are attached to carbon atoms adjacent to each other; or
   L₁ is —CH₂—, oxygen, sulfur or —NR₆— and L₂ is CH which taken together with L₁, R₂ and the carbon atoms to which L₁ and R₂ are attached form a fused 5- to 7-membered ring which may be interrupted with one or two heteroatoms selected from oxygen, nitrogen and sulfur wherein
   R₆ is hydrogen, optionally substituted alkyl, aralkyl, heteroaralkyl, alkoxycarbonyl, aryloxycarbonyl, carbamoyl, sulfonyl or acyl provided that L₁ and R₂ are attached to carbon atoms adjacent to each other;
   L₂ is —(CHR₇)ₙ— wherein
      R₇ is hydrogen, hydroxy, alkoxy, carboxy, optionally substituted alkyl, cycloalkyl, aryl or heteroaryl;
      n is zero or an integer from 1 to 4;
   Z is —(CHR₈)ₘ—, —(CH₂)ₘO(CHR₈)ᵣ—, —(CH₂)ₘS(CHR₈)ᵣ— or —(CH₂)ₘNR₉(CHR₈)ᵣ— wherein
      R₈ is hydrogen, optionally substituted alkyl, cycloalkyl, aryl or heterocyclyl;
      R₉ is hydrogen, optionally substituted alkyl, cycloalkyl, aryl, heterocyclyl, aralkyl, heteroaralkyl, alkoxycarbonyl, aryloxycarbonyl, heteroaryloxycarbonyl, carbamoyl, sulfonyl, acyl or acylamino;
      m and r are independently zero or an integer of 1 or 2;
   Q₁ is hydrogen, optionally substituted alkyl, cycloalkyl, aryl or heterocyclyl provided that
      (i) Q₁ is not 2-phenyloxazol-4-yl when
      R₁ and R₂ are hydrogen;
      X and Y each are CH;
      L₁ is a single bond located at the 4-position;
      L₂ is —(CHR₇)ₙ— wherein n is zero;
      L₃ is —(CHR)ₙ— wherein s is zero;
      Z is —(CH₂)ₘO(CHR₈)ᵣ— wherein R₈ is hydrogen, m is zero and r is 2; and
      Q₂ is oxygen; or
      (ii) Q₁ is not hydrogen when
      R₁ and R₂ are hydrogen;
      X and Y each are CH;
      L₁ is a single bond;
      L₂ is —(CHR₇)ₙ— wherein n is zero;
      L₃ is —(CHR)ₙ— wherein R is hydrogen and s is 1;
      Z is —(CHR₈)ₘ— wherein m is zero; and
      Q₂ is oxygen; or
   Q₁ is —C(O)NR₄ₐR₅ₐ, —C(O)R₁₀, —C(O)OR₁₀ or —S(O)_{q}R₁₀ wherein R₄ₐ and R₅ₐ are as defined for R₄ and R₅; R₁₀ is optionally substituted alkyl, cycloalkyl, aryl, heterocyclyl, aralkyl or heteroaralkyl; q is an integer of 1 or 2; or
   Q₁ is a radical of the formula

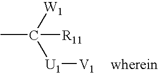

W₁ is aryl, heteroaryl, aralkyl or heteroaralkyl; or
W₁ is —C(O)R₃ₐ in which R₃ₐ is hydroxy or optionally substituted alkoxy; or
   R₃ₐ is —NR₄ₐR₅ₐ in which R₄ₐ and R₅ₐ are as defined for R₄ and R₅;
R₁₁ is hydrogen, alkyl or aryl;
U₁ is —C(O)—, —S(O)₂— or —(CH₂)ᵣ— in which r is as defined for Z;
V₁ is hydroxy, alkoxy, aryl, heteroaryl, optionally substituted alkyl or cycloalkyl; or
V₁ is —NR_{4b}R_{5b} in which R_{4b} and R_{5b} are as defined for R₄ and R₅ provided that
   (i) L₂ is —(CHR₇)ₙ— in which n is an integer of 1 or 2; and
   (ii) Z is —(CHR₈)ₘ— in which m is zero; or
Q₁ is a radical of the formula

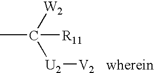

W₂ is —C(O)R₃ₐ in which R₃ₐ is hydroxy or optionally substituted alkoxy; or
   R₃ₐ is —NR₄ₐR₅ₐ in which R₄ₐ and R₅ₐ are as defined for R₄ and R₅;
R₁₁ is hydrogen, alkyl or aryl;
U₂ is —(CH₂)ₚ— in which p is zero or 1;
V₂ is —NR_{4b}C(O)R_{5b}, —NR_{4b}C(O)OR_{5b}, —NR_{4b}C(O)NR_{4c}R_{5b} or —NR_{4b}S(O)₂R_{5b} in which
R_{4b} and R_{4c} are as defined for R₄, and R_{5b} has a meaning as defined for R₅ provided that
   (i) L₂ is —(CHR₇)ₙ— in which n is an integer of 1 or 2; and
   (ii) Z is —(CHR₈)ₘ— in which m is zero; or
Q₁ is a radical of the formula

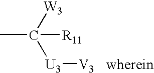

W₃ is —C(O)R₃ₐ in which R₃ₐ is hydroxy or optionally substituted alkoxy; or
   R₃ₐ is —NR₄ₐR₅ₐ in which R₄ₐ and R₅ₐ are as defined for R₄ and R₅;
R₁₁ is hydrogen, alkyl or aryl;
U₃ is —(CH₂)ₚ— in which p is zero or 1;
V₃ is —NHC(O)CHR_{4b}NHC(O)R₁₂ wherein R_{4b} is as defined for R₄; R₁₂ is hydrogen, aryl, heterocyclyl, aralkyl, heteroaralkyl, optionally substituted alkyl, alkoxy or cycloalkyl; or
   R₁₂ is —NR_{4c}R_{5b}, in which R_{4c} and R_{5b} are as defined for R₄ and R₅ provided that
   (i) L₂ is —(CHR₇)ₙ— in which n is an integer of 1 or 2; and
   (ii) Z is —(CHR₈)ₘ— in which m is zero;
L₃ is —(CHR)ₛ— wherein
   R is hydrogen, carboxy, optionally substituted alkyl, cycloalkyl, aryl or heteroaryl;
   s is zero or an integer from 1 to 3;
Q₂ is oxygen, sulfur or NR₁₃ wherein
   R₁₃ is hydrogen, hydroxy or lower alkyl;
X and Y are independently CH or nitrogen; or
   —X═Y— is sulfur, oxygen or —NR₁₄— wherein
      R₁₄ is hydrogen, optionally substituted alkyl, alkoxycarbonyl, acyl, aryloxycarbonyl, heteroaryloxycarbonyl, carbamoyl or sulfonyl;
         or a pharmaceutically acceptable salt thereof; or a prodrug derivative thereof.

Preferred are the compounds of formula (XI) wherein
Q₂ is oxygen;
X and Y each are CH; or
—X═Y— is sulfur;
   or a pharmaceutically acceptable salt thereof; or a prodrug derivative thereof.

Further preferred are the compounds of the formula

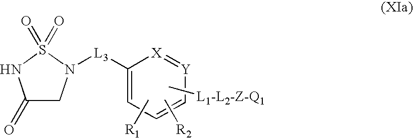

wherein
R₁ is hydrogen, halogen, hydroxy, alkoxy, trifluoromethyl, alkylthio, heteroaralkyl or heteroaralkoxy provided that R₁ is located at the 2-position when L₃ is —(CHR)ₛ— in which s is zero; or
R₁ is optionally substituted alkyl, aralkyl, aralkoxy or aryloxy provided that a monocyclic aryl group which is substituted at the para position with a methylene or ethylene bridged nitrogen containing heterocycle does not constitute part of R₁ when
   (i) R₁ is located at the 2-position and L₃ is —(CHR)ₛ— in which s is zero; and
   (ii) X and Y each are CH;
R₂ is hydrogen; or
R₂ is —C(O)R₃ wherein
   R₃ is hydroxy or optionally substituted alkoxy; or
   R₃ is —NR₄R₅ in which R₄ and R₅ are independently hydrogen, optionally substituted alkyl, cycloalkyl, aryl, heterocyclyl, aralkyl or heteroaralkyl;
L₁ is a single bond; or
L₁ is carbon which combined together with R₂ and the carbon atoms to which L₁ and R₂ are attached form an optionally substituted fused 5- or 6-membered aromatic or heteroaromatic ring provided that L₁ and R₂ are attached to carbon atoms adjacent to each other; or
L₁ is CH or nitrogen which taken together with R₂ and the carbon atoms to which L₁ and R₂ are attached form a fused 5- to 7-membered ring which may be interrupted with one or two heteroatoms selected from oxygen, nitrogen and sulfur provided that L₁ and R₂ are attached to carbon atoms adjacent to each other; or
L₁ is CH, oxygen, sulfur or nitrogen and L₂ is carbon which combined together with L₁, R₂ and the carbon atoms to which L₁ and R₂ are attached form an optionally substituted fused 5- or 6-membered aromatic or heteroaromatic ring provided that L₁ and R₂ are attached to carbon atoms adjacent to each other; or
L₁ is —CH₂—, oxygen, sulfur or —NR₆— and L₂ is CH which taken together with L₁, R₂ and the carbon atoms to which L₁ and R₂ are attached form a fused 5- to 7-membered ring which may be interrupted with one or two heteroatoms selected from oxygen, nitrogen and sulfur wherein
R₆ is hydrogen, optionally substituted alkyl, aralkyl, heteroaralkyl, alkoxycarbonyl, aryloxycarbonyl, carbamoyl, sulfonyl or acyl provided that L₁ and R₂ are attached to carbon atoms adjacent to each other;
L₂ is —(CHR₇)ₙ— wherein
   R₇ is hydrogen;
   n is zero or an integer of 1 or 2;
Z is —(CHR₈)ₘ—, —(CH₂)ₘO(CHR₈)ᵣ—, —(CH₂)ₘS(CHR₈)ᵣ— or —(CH₂)ₘNR₉(CHR₈)ᵣ— wherein
   R₈ is hydrogen or optionally substituted alkyl;
   R₉ is hydrogen, optionally substituted alkyl, cycloalkyl, aryl, heterocyclyl or acyl;
   m and r are independently zero or an integer of 1 or 2;
Q₁ is hydrogen, optionally substituted alkyl, cycloalkyl, aryl or heterocyclyl provided that
   (i) Q₁ is not 2-phenyloxazol-4-yl when
   R₁ and R₂ are hydrogen;
   X and Y each are CH;
   L₁ is a single bond located at the 4-position;
   L₂ is —(CHR₇)ₙ— wherein n is zero;
   L₃ is —(CHR)ₛ— wherein s is zero; and
   Z is —(CH₂)ₘO(CHR₈)ᵣ wherein R₈ is hydrogen, m is zero and r is 2; or
   (ii) Q₁ is not hydrogen when
   R₁ and R₂ are hydrogen;
   X and Y each are CH;
   L₁ is a single bond;
   L₂ is —(CHR₇)ₙ— wherein n is zero;
   L₃ is —(CHR)ₛ— wherein R is hydrogen and s is 1; and
   Z is —(CHR₈)ₘ— wherein m is zero; or
Q₁ is —C(O)NR₄ₐR₅ₐ, —C(O)R₁₀, —C(O)OR₁₀ or —S(O)_{q}R₁₀ wherein R₄ₐ and R₅ₐ are as defined for R₄ and R₅; R₁₀ is optionally substituted alkyl, cycloalkyl, aryl, heterocyclyl, aralkyl or heteroaralkyl; q is an integer of 1 or 2; or
Q₁ is a radical of the formula

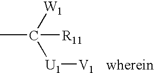

W₁ is aryl, heteroaryl, aralkyl or heteroaralkyl; or
W₁ is —C(O)R₃ₐ in which R₃ₐ is hydroxy or optionally substituted alkoxy; or
   R₃ₐ is —NR₄ₐR₅ₐ in which R₄ₐ and R₅ₐ are as defined for R₄ and R₅;
R₁₁ is hydrogen, alkyl or aryl;
U₁ is —C(O)— or —(CH₂)ᵣ— in which r is as defined for Z;
V₁ is hydroxy, alkoxy, aryl, heteroaryl, optionally substituted alkyl or cycloalkyl; or
V₁ is —NR_{4b}R_{5b} in which R_{4b} and R_{5b} are as defined for R₄ and R₅ provided that
   (i) L₂ is —(CHR₇)ₙ— in which n is an integer of 1 or 2; and
   (ii) Z is —(CHR₈)ₘ— in which m is zero; or
Q₁ is a radical of the formula

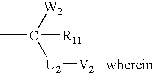

W₂ is —C(O)R₃ₐ in which R₃ₐ is hydroxy or optionally substituted alkoxy; or
   R₃ₐ is —NR₄ₐR₅ₐ in which R₄ₐ and R₅ₐ are as defined for R₄ and R₅;
R₁₁ is hydrogen, alkyl or aryl;
U₂ is —(CH₂)ₚ— in which p is zero or 1;
V₂ is —NR_{4b}C(O)R_{5b}, —NR_{4b}C(O)OR_{5b}, —NR_{4b}C(O)NR_{4c}R_{5b} or —NR_{4b}S(O)₂R_{5b} in which
R_{4b} and R_{4c} are as defined for R₄, and R_{5b} has a meaning as defined for R₅ provided that
   (i) L₂ is —(CHR₇)ₙ— in which n is an integer of 1 or 2; and
   (ii) Z is —(CHR₈)ₘ— in which m is zero; or
Q₁ is a radical of the formula

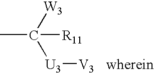

W₃ is —C(O)R₃ₐ in which R₃ₐ is hydroxy or optionally substituted alkoxy; or
   R₃ₐ is —NR₄ₐR₅ₐ in which R₄ₐ and R₅ₐ are as defined for R₄ and R₅;
R₁₁ is hydrogen, alkyl or aryl;
U₃ is —(CH₂)ₚ— in which p is zero or 1;
V₃ is —NHC(O)CHR_{4b}NHC(O)R₁₂ wherein R_{4b} is as defined for R₄; R₁₂ is hydrogen, aryl, heterocyclyl, aralkyl, heteroaralkyl, optionally substituted alkyl, alkoxy or cycloalkyl; or
   R₁₂ is —NR_{4c}R_{5b}, in which R_{4c} and R_{5b} are as defined for R₄ and R₅ provided that
      (i) L₂ is —(CHR₇)ₙ— in which n is an integer of 1 or 2; and
      (ii) Z is —(CHR₈)ₘ— in which m is zero;
L₃ is —(CHR)ₛ— wherein
   R is hydrogen;
   s is zero or an integer from 1 to 3;
X and Y each are CH; or
—X═Y— is sulfur;
   or a pharmaceutically acceptable salt thereof; or a prodrug derivative thereof.

Preferred are the compounds of formula (XIa) of the formula

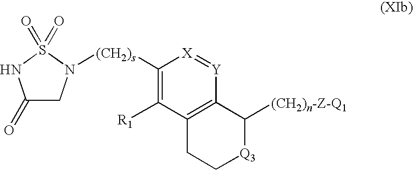

wherein
R₁ is hydrogen, halogen, hydroxy, alkoxy, trifluoromethyl, optionally substituted alkyl, alkylthio, aralkyl, aralkoxy, aryloxy, heteroaralkyl or heteroaralkoxy;
n is zero or an integer of 1 or 2;
Z is —(CHR₈)ₘ—, —(CH₂)ₘO(CHR₈)ᵣ—, —(CH₂)ₘS(CHR₈)ᵣ— or —(CH₂)ₘNR₉(CHR₈)ᵣ— wherein
   R₈ is hydrogen;
   R₉ is hydrogen, optionally substituted alkyl, cycloalkyl, aryl, heterocyclyl or acyl;
   m and r are independently zero or an integer of 1 or 2;
Q₁ is hydrogen, optionally substituted alkyl, cycloalkyl, aryl or heterocyclyl; or
Q₁ is —C(O)NR₄ₐR₅ₐ, —C(O)R₁₀, —C(O)OR₁₀ or —S(O)_{q}R₁₀ wherein
   R₄ₐ and R_{5b} are independently hydrogen, optionally substituted alkyl, cycloalkyl, aryl, heterocyclyl, aralkyl or heteroaralkyl;
   R₁₀ is optionally substituted alkyl, cycloalkyl, aryl, heterocyclyl, aralkyl or heteroaralkyl;
   q is an integer of 1 or 2;
s is zero or an integer of 1 or 2;
Q₃ is O, S or —NR₆ₐ— wherein
   R₆ₐ is hydrogen, optionally substituted alkyl, aralkyl, heteroaralkyl, alkoxycarbonyl, aryloxycarbonyl, carbamoyl, sulfonyl or acyl;
X and Y each are CH; or
—X═Y— is sulfur;
   or a pharmaceutically acceptable salt thereof; or a prodrug derivative thereof.

Preferred are also the compounds of formula (XIa) of the formula

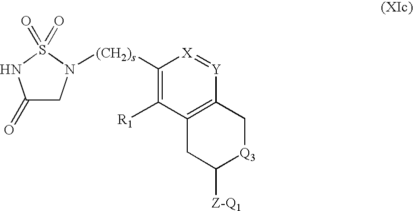

wherein
R₁ is hydrogen, halogen, hydroxy, alkoxy, trifluoromethyl, optionally substituted alkyl, alkylthio, aralkyl, aralkoxy, aryloxy, heteroaralkyl or heteroaralkoxy;
Z is —(CHR₈)ₘ—, —(CH₂)ₘO(CHR₈)ᵣ—, —(CH₂)ₘS(CHR₈)ᵣ— or —(CH₂)ₘNR₉(CHR₈)ᵣ— wherein
   R₈ is hydrogen;
   R₉ is hydrogen, optionally substituted alkyl, cycloalkyl, aryl, heterocyclyl or acyl;
   m and r are independently zero or an integer of 1 or 2;
Q₁ is hydrogen, optionally substituted alkyl, cycloalkyl, aryl or heterocyclyl; or
Q₁ is —C(O)NR₄ₐR₅ₐ, —C(O)R₁₀, —C(O)OR₁₀ or —S(O)_{q}R₁₀ wherein
   R₄ₐ and R₅ₐ are independently hydrogen, optionally substituted alkyl, cycloalkyl, aryl, heterocyclyl, aralkyl or heteroaralkyl;
   R₁₀ is optionally substituted alkyl, cycloalkyl, aryl, heterocyclyl, aralkyl or heteroaralkyl;
   q is an integer of 1 or 2;
s is zero or an integer of 1 or 2;
Q₃ is O, S or —NR₆ₐ— wherein
   R₆ₐ is hydrogen, optionally substituted alkyl, aralkyl, heteroaralkyl, alkoxycarbonyl, aryloxycarbonyl, carbamoyl, sulfonyl or acyl;
X and Y each are CH; or
—X═Y— is sulfur;
   or a pharmaceutically acceptable salt thereof; or a prodrug derivative thereof.

Preferred are also the compounds of formula (XIa) wherein
R₂ is hydrogen;
L₁ is a single bond;
L₂ is —(CH₂)ₙ— in which n is zero or an integer of 1 or 2;
   or a pharmaceutically acceptable salt thereof; or a prodrug derivative thereof.

Further preferred are the compounds of formula (XIa) of the formula

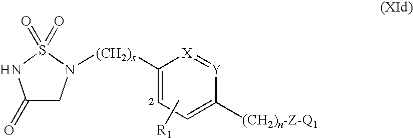

wherein
R₁ is hydrogen, halogen, hydroxy, alkoxy, trifluoromethyl or alkylthio provided that R₁ is located at the 2-position when s is zero; or
R₁ is optionally substituted alkyl, aralkyl, aralkoxy or aryloxy provided that a monocyclic aryl group which is substituted at the para position with a methylene or ethylene bridged nitrogen containing heterocycle does not constitute part of R₁ when
   (i) R₁ is located at the 2-position and s is zero; and
   (ii) X and Y each are CH;
n is zero or an integer of 1 or 2;
s is zero or 1;
Z is —(CHR₈)ₘ—, —(CH₂)ₘO(CHR₈)ᵣ—, —(CH₂)ₘS(CHR₈)ᵣ— or —(CH₂)ₘNR₉(CHR₈)ᵣ— wherein
   R₈ is hydrogen;
   R₉ is hydrogen, optionally substituted alkyl, cycloalkyl, aryl, heteroaryl or acyl;
   m and r are independently zero or an integer of 1 or 2;
Q₁ is hydrogen, optionally substituted alkyl, cycloalkyl, aryl or heterocyclyl provided that
   (i) Q₁ is not 2-phenyloxazol-4-yl when
   R₁ is hydrogen;
   X and Y each are CH;
   n is zero;
   s is zero; and
   Z is —(CH₂)ₘO(CHR₈)ᵣ— wherein R₈ is hydrogen, m is zero and r is 2; or
   (ii) Q₁ is not hydrogen when
   R₁ is hydrogen;
   X and Y each are CH;
   n is zero;
   s is 1;
   Z is —(CHR₈)ₘ— wherein m is zero; or
Q₁ is —C(O)NR₄ₐR₅ₐ, —C(O)R₁₀, —C(O)OR₁₀ or —S(O)_{q}R₁₀ wherein
   R₄ₐ and R₅ₐ are independently hydrogen, optionally substituted alkyl, cycloalkyl, aryl, heterocyclyl, aralkyl or heteroaralkyl;
   R₁₀ is optionally substituted alkyl, cycloalkyl, aryl, heterocyclyl, aralkyl or heteroaralkyl;
   q is an integer of 1 or 2; or
Q₁ is a radical of the formula

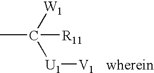

W₁ is aryl, heteroaryl, aralkyl or heteroaralkyl; or
W₁ is —C(O)R₃ₐ in which R₃ₐ is hydroxy or optionally substituted alkoxy; or
   R₃ₐ is —NR₄ₐR₅ₐ in which R₄ₐ and R₅ₐ are independently hydrogen, optionally substituted alkyl, cycloalkyl, aryl, heterocyclyl, aralkyl or heteroaralkyl;
R₁₁ is hydrogen, alkyl or aryl;
U₁ is —C(O)— or —(CH₂)ᵣ in which r is as defined for Z;
V₁ is hydroxy, alkoxy, aryl, heteroaryl, optionally substituted alkyl or cycloalkyl; or
V₁ is —NR_{4b}R_{5b} in which R_{4b} and R_{5b} are as defined for R₄ₐ and R₅ₐ provided that
   (i) n is an integer of 1 or 2; and
   (ii) Z is —(CHR₈)ₘ— in which m is zero; or
Q₁ is a radical of the formula

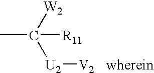

W₂ is —C(O)R₃ₐ in which R₃ₐ is hydroxy or optionally substituted alkoxy; or
   R₃ₐ is —NR₄ₐR₅ₐ in which R₄ₐ and R₅ₐ are independently hydrogen, optionally substituted alkyl, cycloalkyl, aryl, heterocyclyl, aralkyl or heteroaralkyl;
R₁₁ is hydrogen, alkyl or aryl;
U₂ is —(CH₂)ₚ— in which p is zero or 1;
V₂ is —NR_{4b}C(O)R_{5b}, —NR_{4b}C(O)OR_{5b}, —NR_{4b}C(O)NR_{4c}R_{5b} or —NR_{4b}S(O)₂R_{5b} in which
R_{4b} and R_{4c} are as defined for R₄ₐ, and R_{5b} has a meaning as defined for R₅ₐ provided that
   (i) n is an integer of 1 or 2; and
   (ii) Z is —(CHR₈)ₘ— in which m is zero; or
Q₁ is a radical of the formula

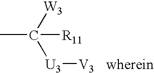

W₃ is —C(O)R₃ₐ in which R₃ₐ is hydroxy or optionally substituted alkoxy; or
   R₃ₐ is —NR₄ₐR₅ₐ in which R₄ₐ and R₅ₐ are independently hydrogen, optionally substituted alkyl, cycloalkyl, aryl, heterocyclyl, aralkyl or heteroaralkyl;
R₁₁ is hydrogen, alkyl or aryl;
U₃ is —(CH₂)ᵣ— in which r is zero or 1;
V₃ is —NHC(O)CHR_{4b}NHC(O)R₁₂ wherein R_{4b} is as defined for R₄ₐ; R₁₂ is hydrogen, aryl, heterocyclyl, aralkyl, heteroaralkyl, optionally substituted alkyl, alkoxy or cycloalkyl; or
   R₁₂ is —NR_{4c}R_{5b} in which R_{4c} is as defined for R₄ₐ, and R_{5b} has a meaning as defined for R₅ₐ provided that
   (i) n is an integer of 1 or 2; and
   (ii) Z is —(CHR₈)ₘ— in which m is zero;
X and Y each are CH; or
—X═Y— is sulfur;
   or a pharmaceutically acceptable salt thereof; or a prodrug derivative thereof.

Preferred are the compounds of formula (XId) wherein
—X═Y— is sulfur;
   or a pharmaceutically acceptable salt thereof; or a prodrug derivative thereof.

Preferred are also the compounds of formula (XId) wherein
R₁ is bromide;
X and Y each are CH;
   or a pharmaceutically acceptable salt thereof; or a prodrug derivative thereof.

Preferred are also the compounds of formula (XId) wherein
n is zero;
s is 1;
Z is —(CH₂)ₘ— in which m is zero;
Q₁ is —C(O)NR₄ₐR₅ₐ, —C(O)R₁₀, —C(O)OR₁₀ or —S(O)_{q}R₁₀ wherein
   R₄ₐ and R₅ₐ are independently hydrogen, optionally substituted alkyl, cycloalkyl, aryl, heterocyclyl, aralkyl or heteroaralkyl;
   R₁₀ is optionally substituted alkyl, cycloalkyl, aryl, heterocyclyl, aralkyl or heteroaralkyl;
   q is an integer of 1 or 2;
      or a pharmaceutically acceptable salt thereof; or a prodrug derivative thereof.

Preferred are also the compounds of formula (XId) wherein
n is an integer of 1 or 2;
Z is —(CH₂)ₘ—, —(CH₂)ₘO(CH₂)ᵣ— or —(CH₂)ₘS(CH₂)ᵣ— wherein
   m is zero;
   r is zero or 1;
      Q₁ is optionally substituted alkyl, cycloalkyl, aryl or heterocyclyl;
         or a pharmaceutically acceptable salt thereof; or a prodrug derivative thereof.

Preferred are also the compounds of formula (XId) wherein
n is an integer of 1 or 2;
Z is —(CH₂)ₘNR₉(CH₂)ᵣ— wherein
   R₉ is hydrogen, optionally substituted alkyl, cycloalkyl, aryl, heteroaryl or acyl;
   m is zero;
   r is zero or 1;
Q₁ is optionally substituted alkyl, cycloalkyl, aryl or heterocyclyl; or
Q₁ is —C(O)NR₄ₐR₅ₐ, —C(O)R₁₀, —C(O)OR₁₀ or —S(O)_{q}R₁₀ wherein
   R₄ₐ and R₅ₐ are independently hydrogen, optionally substituted alkyl, cycloalkyl, aryl, heterocyclyl, aralkyl or heteroaralkyl;
   R₁₀ is optionally substituted alkyl, cycloalkyl, aryl, heterocyclyl, aralkyl or heteroaralkyl;
   q is an integer of 1 or 2;
      or a pharmaceutically acceptable salt thereof; or a prodrug derivative thereof.

Preferred are also the compounds of formula (XId) wherein
n is an integer of 1 or 2;
Z is —(CH₂)ₘ— wherein m is zero;
Q₁ is a radical of the formula

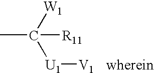

W₁ is aryl, heteroaryl, aralkyl or heteroaralkyl;
R₁₁ is hydrogen, alkyl or aryl;
U₁ is —C(O)— or —(CH₂)ᵣ— in which r is zero;
V₁ is aryl, heteroaryl, optionally substituted alkyl or cycloalkyl;
   or a pharmaceutically acceptable salt thereof; or a prodrug derivative thereof.

Preferred are also the compounds of formula (XId) wherein
n is 1;
Z is —(CH₂)ₘ— wherein m is zero;
Q₁ is a radical of the formula

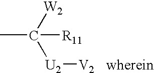

W₂ is —C(O)R₃ₐ in which R₃ₐ is —NR₄ₐR₅ₐ and R₄ₐ and R₅ₐ are independently hydrogen, optionally substituted alkyl, cycloalkyl, aryl, heterocyclyl, aralkyl or heteroaralkyl;
R₁₁ is hydrogen;
U₂ is —(CH₂)ₚ— in which p is zero;
V₂ is —NR_{4b}C(O)R_{5b}, —NR_{4b}C(O)OR_{5b}, —NR_{4b}C(O)NR_{4c}R_{5b} or —NR_{4b}S(O)₂R_{5b} in which
R_{4b} and R_{4c} are as defined for R₄ₐ, and R_{5b} has a meaning as defined for R₅ₐ;
   or a pharmaceutically acceptable salt thereof; or a prodrug derivative thereof.

Preferred are also the compounds of formula (XId) wherein
n is 1;
Z is —(CH₂)ₘ— wherein m is zero;
Q₁ is a radical of the formula

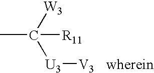

W₃ is —C(O)R₃ₐ in which R₃ₐ is —NR₄ₐR₅ₐ and R₄ₐ and R₅ₐ are independently hydrogen, optionally substituted alkyl, cycloalkyl, aryl, heterocyclyl, aralkyl or heteroaralkyl;
R₁₁ is hydrogen;
U₃ is —(CH₂)ₚ— in which p is zero;
V₃ is —NHC(O)CHR_{4b}NHC(O)R₁₂ wherein R_{4b} is as defined for R₄ₐ; R₁₂ is hydrogen, aryl, heterocyclyl, aralkyl, heteroaralkyl, optionally substituted alkyl or alkoxy; or
   R₁₂ is —NR_{4c}R_{5b} in which R_{4c} and R_{5b} are as defined for R₄ₐ and R₅ₐ;
      or a pharmaceutically acceptable salt thereof; or a prodrug derivative thereof.

Methods of preparing the above compounds are disclosed in WO 2003082841 published Oct. 9, 2003, which is incorporated herein by reference in its entirety.

As indicated herein above, the compounds to be combined may be present as their pharmaceutically acceptable salts. If these compounds have, e.g., at least one basic center such as an amino group, they can form acid addition salts thereof. Similarly, the compounds having at least one acid group (for example COOH) can form salts with bases. Corresponding internal salts may furthermore be formed, if a compound comprises, e.g., both a carboxy and an amino group.

The corresponding active ingredients or a pharmaceutically acceptable salts may also be used in form of a solvate, such as a hydrate or including other solvents used, e.g., in their crystallization.

Furthermore, the present invention provides pharmaceutical compositions comprising a renin inhibitor, or a pharmaceutically acceptable salt thereof, and at least one therapeutic agent selected from the group consisting of

(a) an insulin secretion enhancer, or a pharmaceutically acceptable salt thereof; and
(b) an insulin sensitizer, or a pharmaceutically acceptable salt thereof; and a pharmaceutically acceptable carrier.

A pharmaceutical composition according to the present invention may be employed for the prevention of, delay the onset of or treatment of a disease or a condition modulated by the inhibition of renin activity and/or insulin secretion enhancer and/or insulin sensitizer.

As disclosed herein above, a renin inhibitor, in particular, aliskiren, preferably in the form of the hemi-fumarate salt thereof, and at least one therapeutic agent selected from the group consisting of an insulin secretion enhancer, or a pharmaceutically acceptable salt thereof, and an insulin sensitizer, or a pharmaceutically acceptable salt thereof, may be co-administered as a pharmaceutical composition. The components may be administered together in any conventional dosage form, usually also together with a pharmaceutically acceptable carrier or diluent.

The pharmaceutical compositions according to the invention are those suitable for enteral, such as oral or rectal, transdermal and parenteral administration to mammals, including man. For oral administration the pharmaceutical composition comprising a renin inhibitor, in particular, aliskiren, preferably in the form of the hemi-fumarate salt thereof, and at least one therapeutic agent selected from the group consisting of an insulin secretion enhancer, or a pharmaceutically acceptable salt thereof, and an insulin sensitizer, or a pharmaceutically acceptable salt thereof, can take the form of solutions, suspensions, tablets, pills, capsules, powders, microemulsions, unit dose packets and the like. Preferred are tablets and gelatin capsules comprising the active ingredient together with: a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbants, colorants, flavors and sweeteners. Injectable compositions are preferably aqueous isotonic solutions or suspensions, and suppositories are advantageously prepared from fatty emulsions or suspensions.

Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-90%, preferably about 1-80%, of the active ingredient.

The dosage of the active ingredients can depend on a variety of factors, such as mode of administration, homeothermic species, age and/or individual condition.

Normally, in the case of oral administration, an approximate daily dose of from about 1 mg to about 360 mg is to be estimated e.g. for a patient of approximately 75 kg in weight.

For example, the doses of aliskiren to be administered to warm-blooded animals, including man, of approximately 75 kg body weight, especially the doses effective for the inhibition of renin activity, e.g., in lowering blood pressure, are from about 3 mg to about 3 g, preferably from about 10 mg to about 1 g, e.g., from 20 to 200 mg/person/day, divided preferably into 1 to 4 single doses which may, e.g., be of the same size. Usually, children receive about half of the adult dose. The dose necessary for each individual can be monitored, e.g., by measuring the serum concentration of the active ingredient, and adjusted to an optimum level. Single doses comprise, e.g., 75 mg, 150 mg or 300 mg per adult patient.

The insulin secretion enhancer GKA2 is preferably administered to the warm-blooded animal in a dosage in the range of about 0.1 to 1500 mg/day, more preferably 25 to 800 mg/day, when the warm-blooded animal is a human of about 70 kg body weight. Preferred dosages contain 30 mg, 60 mg, 120 mg or 180 mg of GKA2 to be administered preferably before the main meals. In a low dose combination, the dosage of to be administered preferably is 30 mg, 40 mg or furthermore 60 mg. Depending on the number of main meals the dose regimen are two times a day (BID) or three times a day (TID) or four times a day (QID).

The insulin secretion enhancer GKA2 is preferably administered in a dosage range of about 0.01 mg to about 8 mg, more preferred from about 0.5 to about 6 mg.

The present invention further relates to a method for the prevention, delay the onset of or treatment of a disease or a condition which may be modulated by the inhibition of renin activity and/or an insulin secretion enhancer and/or an insulin sensitizer comprising administering to a warm-blooded animal, including man, in need thereof, a therapeutically effective amount of a combination comprising a renin inhibitor, in particular, aliskiren, or a pharmaceutically acceptable salt thereof, and at least one therapeutic agent selected from the group consisting of

(a) an insulin secretion enhancer, or a pharmaceutically acceptable salt thereof; and
(b) an insulin sensitizer, or a pharmaceutically acceptable salt thereof.

Accordingly, the combination according to the present invention may be used, e.g., for the prevention, delay the onset of or treatment of diseases and disorders that may be modulated by the inhibition of renin activity, and/or by insulin secretion enhancer and/or by insulin sensitizer. Especially, the combination according to the present invention may be used, e.g., for the prevention of, delay the onset of or the treatment of diseases and conditions selected from the group consisting of hypertension, congestive heart failure, diabetes, especially type 2 diabetes mellitus, mature onset diabetes of the young (MODY), diabetic retinopathy, macular degeneration, diabetic nephropathy, hypertensive or non-hypertensive nephropathy, IgA nephropathy, glomerulosclerosis, chronic renal failure, diabetic neuropathy, metabolic syndrome, cardiac syndrome X, atherosclerosis, coronary heart disease, angina pectoris, myocardial infarction, stroke, coronary and vascular restenosis, hyperglycemia, hyperinsulinaemia, hyperlipidaemia, hypertryglyceridemia, insulin resistance, impaired glucose metabolism (IGM), conditions of impaired glucose tolerance (IGT), IGM and/or IGT or increased inflammation in women with polycystic ovary syndrome or women with prior gestational diabetes, conditions of impaired fasting plasma glucose, obesity, diabetic retinopathy, macular degeneration, cataracts, foot ulcerations, endothelial dysfunction, impaired vascular compliance and obstructive sleep apnea. Preferably, the said combination may be used for the treatment of hypertension, including ISH, as well as congestive heart failure, metabolic syndrome, endothelial dysfunction, impaired vascular compliance, IGT, diabetes especially type 2 diabetes mellitus, hypertensive or non-hypertensive nephropathy, IgA nephropathy, as well as retardation or prolongation of the progression of prediabetes to diabetes.

Preferably, the combined therapeutically effective amounts of the active agents according to the combination of the present invention can be administered simultaneously or sequentially in any order, e.g., separately or in a fixed combination.

Additionally, the present invention relates to the use of a combination comprising a renin inhibitor, in particular, aliskiren, or a pharmaceutically acceptable salt thereof, and at least one therapeutic agent selected from the group consisting of

(a) an insulin secretion enhancer, or a pharmaceutically acceptable salt thereof; and
(b) an insulin sensitizer, or a pharmaceutically acceptable salt thereof;
and a pharmaceutically acceptable carrier; for the manufacture of a medicament for the prevention, delay the onset of or treatment of a disease or a condition which may be modulated by the inhibition of renin activity and/or by an insulin secretion enhancer and/or an insulin sensitizer.

The pharmaceutical composition according to the present invention as described hereinbefore and hereinafter may be used for simultaneous use or sequential use in any order, for separate use or as a fixed combination.

Preferred are combinations, such as a combined preparations or pharmaceutical compositions, respectively, comprising aliskiren, or a pharmaceutically accepted salt thereof, and at least one therapeutic agent selected from the group consisting of (i) GKA2 or another agent with activating effect on GK, and (ii) an agent with inhibitory effects on SCD-1.

Since the present invention has an aspect that relates to methods for the prevention of, delay the onset of or treatment with a combination of compounds which may be administered separately, the invention also relates to combining separate pharmaceutical compositions in a kit form. Accordingly, the pharmaceutical composition according to the present invention may comprise a “kit of parts” in the sense that the components can be dosed independently or by use of different fixed combinations with distinguished amounts of the components at different time points. The parts of the “kit of parts” can then, e.g., be administered simultaneously or chronologically staggered, that is at different time points and with equal or different time intervals for any part of the “kit of parts”. Preferably, the time intervals are chosen such that the effect on the treated disease or condition in the combined use of the parts is larger than the effect that would be obtained by use of only any one of the components. Preferably, there is at least one beneficial effect, e.g., a mutual enhancing of the efficacy of a renin inhibitor, e.g., aliskiren, or a pharmaceutically acceptable salt thereof, and at least one therapeutic agent selected from the group consisting of

(a) an insulin secretion enhancer, or a pharmaceutically acceptable salt thereof; and
(b) an insulin sensitizer, or a pharmaceutically acceptable salt thereof;
in particular, a potentiation or a synergism, e.g., a more than additive effect, additional advantageous effects, less side effects, a combined therapeutical effect in a non-effective dosage of one or each of the components, especially, a potentiation or a strong synergism.

The term “potentiation” shall mean an increase of a corresponding pharmacological activity or therapeutical effect, respectively. Potentiation of one component of the combination according to the present invention by co-administration of another component according to the present invention means that an effect is being achieved that is greater than that achieved with one component alone.

The term “synergistic” shall mean that the drugs, when taken together, produce a total joint effect that is greater than the sum of the effects of each drug when taken alone.

The invention furthermore relates to a commercial package comprising the combination according to the present invention together with instructions for simultaneous, separate or sequential use.

The pharmaceutical activities as effected by administration of a renin inhibitor, in particular, aliskiren, or a combination of the active agents used according to the present invention can be demonstrated, e.g., by using corresponding pharmacological models known in the pertinent art. The person skilled in the pertinent art is fully enabled to select a relevant animal test model to prove the hereinbefore and hereinafter indicated therapeutic indications and beneficial effects.

To evaluate the antihypertensive activity of the combination according to the invention, for example, the methodology as described by Lovenberg W: Animal models for hypertension research. Prog. Clin. Biol. Res. 1987, 229, 225-240 may be applied. For the evaluation that the combination according to the present invention may be used for the treatment of congestive heart failure, for example, the methods as disclosed by Smith H J, Nuttall A: Experimental models of heart failure. Cardiovasc Res 1985, 19, 181-186 may be applied. Molecular approaches such as transgenic methods are also described, for example by Luft et al.: Hypertension-induced end-organ damage, “A new transgemic approach for an old problem”, Hypertension 1999, 33, 212-218.

The insulin secretion enhancing properties of the combination according to the present invention may be determined by following the methodology as disclosed, for example, in the publication of Ikenoue et al. Biol. Pharm. Bull. 29(4), 354-359 (1997).

The simultaneous evaluation of the cardiovascular actions and glucose utilization effects of the agents given alone or in combination can be performed using models such as the Zucker fatty rat as described in the publication of Nawano et al., Metabolism 48: 1248-1255, 1999. Also, studies using diabetic spontaneously hypertensive rats are described in the publication of Sato et al., Metabolism 45:457-462, 1996.

The insulin secretion enhancing properties of the combination according to the present invention may be determined by following the methodology as disclosed, for example, in the publication of Ikenoue et al. Biol. Pharm. Bull. 29(4), 354-359 (1997). Hypertension, in connection with a “disease or condition which may be modulated by the inhibition of renin activity”, a “disease or condition which may be modulated by the insulin secretion enhancer”, a “disease or condition that may be modulated by insulin sensitizer” includes, but is not limited to, mild, moderate and severe hypertension as defined in Journal of Hypertension 1999, 17:151-183, especially, on page 162 and is inclusive of Isolated Systolic Hypertension (ISH).

Under certain circumstances, drugs with different mechanisms of action may be combined. However, just considering any combination of drugs having different modes of action but acting in the similar field does not necessarily lead to combinations with advantageous effects.

All the more surprising is the experimental finding that the combined administration of a renin inhibitor, in particular, aliskiren, and an insulin secretion enhancer and/or an insulin sensitizer, or, in each case, a pharmaceutically acceptable form thereof, results not only in a beneficial, especially a potentiating or a synergistic therapeutic effect, but independent thereof, additional benefits resulting from combined treatment can be achieved such as a surprising prolongation of efficacy, decreased time necessary to achieve efficacy, a broader variety of therapeutic treatment and surprising beneficial effects on diseases and conditions associated with diabetes, e.g., less weight gain, delayed progression of microalbuminuria to frank proteinuria and reduction in albuminuria levels as determined by 24-hour urine analysis. An additional and preferred aspect of the present invention is the prevention, delay the onset of and/or treatment of the condition of isolated systolic hypertension and impaired vascular compliance which means decreased vascular elasticity or arterial compliance.

Guidelines issued jointly by the European Society of Hypertension and the European Society of Cardiology as well as the seventh report of the Joint National Committee (JNC-7) on prevention, detection, evaluation and treatment of high blood pressure emphasize the need to achieve target blood pressure goals of <140/90 mmHg in the general population and even lower levels of blood pressure for patients with diabetes and renal disease. Data from the National Health and Nutrition Examination Survey indicate that only 55 percent of hypertensive patients in the United States receive treatment, and only 29 percent are controlled at a blood pressure below 140/90 mmHg. Many reasons exist for inadequate blood pressure control including patient compliance, reluctance of physicians to titrate medications to their appropriate levels, concerns over adverse events, and treatment costs. New treatment options for physicians and patients may help to address some of these issues.

Isolated Systolic Hypertension (ISH) is the most common form of hypertension in the elderly. It is defined as elevated systolic blood pressure (above 140 mmHg) in conjunction with normal diastolic blood pressure (below 90 mmHg). Elevated systolic blood pressure is an independent risk factor for cardiovascular diseases and may lead e.g. to myocardial hypertrophy and heart failure. ISH is furthermore characterized by an increased pulse pressure, defined as the difference between systolic and diastolic blood pressures. Elevated pulse pressure is being recognized as the type of hypertension the least likely to be well controlled. A reduction of elevated systolic blood pressure and correspondingly of pulse pressure is associated with a significant risk reduction in cardiovascular death. It has surprisingly been found that the combination of a renin inhibitor, in particular, aliskiren, and an insulin secretion enhancer or an insulin sensitizer results in enhanced reduction of elevated diastolic and/or systolic blood pressure in patients with different forms of hypertension including ISH. This combination has also been found to reduce the pulse pressure both in hypertensive patients having type 2 diabetes mellitus and in hypertensive patients that do not have type 2 diabetes mellitus.

Furthermore, it has been found that the chronic co-administration of either an insulin sensitizer or an insulin secretion enhancer with a renin inhibitor, in particular, aliskiren, imparts the beneficial effect on blood vessel morphology and function and results in a decrease of vascular stiffness and correspondingly in a maintenance and in an improvement of vascular compliance, primarily arterial compliance.

Accordingly, it has been found that the addition of an insulin sensitizer and/or an insulin secretion enhancer to that of a renin inhibitor, in particular, aliskiren, would potentiate the effect on systolic blood pressure and further improve vascular stiffness/compliance. Conversely, the proven antihypertensive effects of a renin inhibitor, in particular, aliskiren, on systolic and diastolic blood pressure may be potentiated by the addition of an insulin sensitizer and/or an insulin secretion enhancer. The benefit of these combinations may also extend to an additional or potentiated effect on endothelial function, and improvement of vascular function and structure in various organs/tissues including the kidney, heart, eye and brain. Through the reduction in glucose levels, an anti-thrombotic and anti-atherosclerotic effect can also be demonstrated. Reduction of glucose would prevent or minimize the glycosylation of any structural or functional protein within the cardio-renal system. This effect proves to be highly beneficial by evoking an additive or synergistic effect on vascular function/structure when administered with a renin inhibitor, in particular, aliskiren, which alone improves cardiovascular function and structure through a distinct mechanism.

Additionally, insulin resistance may contribute, in part, to the development of diabetes, hypertension and atherosclerosis (Fukuda et al., 2001). it is known that angiotensin II impairs insulin signaling (Fukuda et al., 2001) and that interruption of the renin angiotensin system with the use of an ACE inhibitor can partially restore insulin sensitivity (Sato et al., 1996; Nawano et al., 1999). Insulin can produce vasodilatation and lower blood pressure (Baron and Steinberg, 1996). The Zucker fatty rat, an animal model with insulin resistance, has been shown to possess a significantly higher blood pressure (Alonso-Galicia et al., 1996). ACE inhibition lowers blood pressure and improves insulin sensitivity in this model (Nawano et al., 1999). Combined administration of a renin inhibitor with either an insulin sensitizer or an insulin secretion enhancer will evoke further antihypertensive effects, improve vascular dynamics in hypertensive patients to a greater extent than after administration of either agent given alone. Interestingly, the co-administration of a renin inhibitor and either an insulin sensitizing agent or an insulin secretion enhancer will partially restore insulin sensitivity by preventing renin angiotensin system-induced impairment of insulin signaling pathways while at the same time raise insulin levels and improve glucose utilization. Consequently, combined administration will simultaneously improve both the metabolic and cardiovascular abnormalities, two conditions that often coexist in patients.

Further benefits include the use of lower doses of the individual drugs to be combined according to the present invention, when compared to the doses used when administered alone, for example, that the dosages could not only often be lower but are also given less frequently, thereby reducing the incidence of side effects. This is in accordance with the desires and requirements of the patients to be treated.

For example, it has been observed that the combination according to the present invention provides benefit especially in the treatment of mild to moderate hypertension or isolated systolic hypertension in patients with prediabetes or diabetes, regardless of their hypertensive status, e.g., by reducing the risk of negative cardiovascular events by two different modes of action.

For example, the renin inhibitor aliskiren has proven to be useful in the treatment of type 2 diabetes mellitus beyond the reduction of blood pressure in for example improving microalbuminuria. At sub-therapeutic doses, with respect to the treatment of hypertension, the combination according to the invention may be merely used for the treatment of diabetes, especially type 2 diabetes mellitus. In view of the reduced dose of aliskiren, there is a considerable safety profile of the combination making it suitable for a first line therapy.

### Clinical Study Design:

I. Study in Subjects with Impaired Glucose Tolerance

A 3 month study is carried out in patients of either sex of age 18 and above (female patients are either surgically sterile or exercise barrier method of birth control with spermicide for the study duration) with an established diagnosis of mild to moderate essential hypertension and an impaired OGTT test confirmatory of impaired glucose tolerance (IGT) which is considered to represent pre-diabetes. IGT is defined as a 2-hour plasma glucose level of >140 mg/dl and <200 mg/dl after a 75 g glucose-equivalent oral glucose challenge in subjects who are not receiving hypoglycemic therapy. A total of 45 eligible patients who meet the OGTT and other entry criteria are randomized in a 1:1:1 ratio into 3 groups each treated for a total of 12 weeks respectively with: (i) renin inhibitor of formula (1), (ii) an insulin secretion enhancer or (iii) a combination of the renin inhibitor of formula (1) and an insulin secretion enhancer. Any hypertensive patient not receiving the renin inhibitor may receive an antihypertensive agent of any class other than an angiotensin converting enzyme inhibitor or and angiotensin receptor blocker. The following assays are performed to detect improvements in glucose tolerance:

### Impaired Glucose Tolerance and Insulin Sensitivity Assays:

The Oral Glucose Tolerance Test (OGTT) is administered at baseline and at weeks 12 and 24. Subjects are given a 75 g glucose-equivalent oral glucose challenge. Venous blood samples are taken for the determination of plasma glucose and serum insulin at time points 0, 30, 60, 90, 120, and 180 min after glucose load. After a 10-h overnight fast, an oral glucose tolerance test (OGTT) is performed commencing between 08:00 and 10:00 by orally administering a solution of 75 g of glucose and 150 ml of free water. Venous blood samples are obtained for determining plasma glucose, insulin and c-peptide concentrations at 0, 30, 60, 90, and 120 min after glucose ingestion. The glucose, insulin and c-peptide area under curve (AUC) in response to OGTT are determined. The insulinogenic index (measure of insulin production during the OGTT) is calculated as the total increase in plasma insulin level divided by the total increase in plasma glucose during the 2-h period of OGTT.

### Results:

The glucose, insulin, and c-peptide areas under curve (AUC) in response to OGTT and the glucose and insulin AUC during meal are improved in the insulin secretion enhanser group (group ii) at 12 weeks, but not at 8 weeks. These values, however are not significantly improved at either 8 or 12 weeks in the renin inhibitor group (group i). Surprisingly, the combination of the renin inhibitor and the insulin secretion enhancer (group iii) not only shows significant improvement in all measurements at both 8 and 12 weeks, but at 12 weeks the combination results in a greater than additive effect in response to OGTT compared with either of the monotherapies (groups i or ii).

II. Study in Subjects with Type 2 Diabetes and Proteinuria

A 24 week study is carried out in diabetic patients of either sex of age 18 and above (female patients are either surgically sterile or exercise barrier method of birth control with spermicide for the study duration). Patients are required to have type 2 diabetes mellitus and have evidence of persistent microalbuminuria (median urinary albumin excertion rate [UAER] of 2 nonconsecutive overnight urine collections to be in the range of 20 to 200 μg/min during the month prior to study entry). Exclusion criteria include, but are not restricted to: abnormal serum creatinine, Type 1 diabetes, use of insulin within 6 months prior to randomization, use of or angiotensin converting enzyme inhibitors or angiotensin receptor blockers within the 4 weeks prior to randomization, heart failure, history of myocardial infarction, PTCA or cerebrovascular accident within the preceding 3 months; and severe diabetic neuropathy.

Subjects are randomized in a 1:1:1 ratio into 3 groups each receiving treatment for 24 weeks respectively with: (i) renin inhibitor of formula (1), (ii) an insulin secretion enhancer, or (iii) a combination of the renin inhibitor of formula (1) and the insulin secretion enhancer. Any hypertensive subject not randomized to receive the renin inhibitor may receive an antihypertensive agent of any class other than an angiotensin converting agent inhibitor or an angiotensin receptor blocker. Any diabetic subject not randomized to receive the insulin secretion enhancer may receive acarbose. The following assays are performed to detect improvements in proteinuria or arterial compliance.

### Proteinuria Assays:

24-hour urine collections are collected at baseline, week 12 and week 24 and examined for urea, creatinine, phosphate, sodium, potassium, and total proteins. Albuminuria and creatinine clearance are measured. Aliquots of validated 24 h urine collections are centrifuged for 5 min to remove cells and particulate matter, and the supernates are treated with 1 mM phenylmethylsulfonyl fluoride (PMSF) and stored at −20° C. Samples are thawed rapidly and centrifuged for 5 min at 2000 r.p.m. to remove any urates or phosphates before assays are performed. UAER and UACR are calculated. Arterial compliance measurements: Arterial distensibility is assessed by automatic carotid-femoral pulse wave velocity measurement (PWV) at baseline, 12 weeks and 24 weeks. The basic principle of PWV assessment is that the pressure pulse generated by ventricular ejection is propagated along the arterial tree at a speed determined by the geometric and elastic properties of the arterial wall. PWV is calculated from measurements of pulse transit time and the distance traveled by the pulse between two recording sites, according to the following formula: PWV (m/s)=distance (m)/transit time (s). Carotid-femoral PWV is calculated from the time delay between the recorded proximal (carotid) and distal (femoral) feet of the wave, and the superficially measured distance separating the respective transducers.

### Results:

The UAER is improved at 24 weeks compared to baseline in both groups receiving renin inhibition (groups i and iii), however no improvement is observed at 24 weeks in the group receiving only the insulin secretion enhanser (group ii). Surprisingly, the combination of the renin inhibitor and the insulin secretion enhancer (group iii) not only shows significant improvement in all measurements at both 12 and 24 weeks, but in addition, the combination results in a greater than additive effect on proteinuria. In addition, and also surprising, only the combination of the renin inhibitor and the insulin secretion enhancer (group iii) demonstrates a significant proportion of patients returning to normoalbuminuria status (UAER<20 μg/min at the last visit measurement).

Also surprising, arterial compliance, as measured by pulse wave velocity, is improved at the 24 week time point only by treatment with the combination of the renin inhibitor and the insulin secretion enhancer. Neither treatment with the renin inhibitor alone (group i) nor treatment with the insulin secretion enhancer alone (group ii) demonstrates a significant effect on pulse wave velocity.

### EXAMPLE 1

Composition of aliskiren 150 mg (free base) uncoated tablets in mg/unit.

| | Roller | | | |
|---|---|---|---|---|
| | compacted | Dosage | Dosage | Dosage |
| Component | tablet | form 1 | form 2 | form 3 |
| Aliskiren hemi-fumarate | 165.750 | 165.750 | 165.750 | 165.750 |
| Microcrystalline cellulose | 220.650 | 84.750 | 72.250 | 107.250 |
| Polyvinylpyrrolidon K 30 | — | — | 12.000 | 12.000 |
| Crospovidone | 84.000 | 45.000 | 44.000 | 48.200 |
| Aerosil 200 | 4.800 | 1.500 | 1.500 | 1.800 |
| Magnesium stearate | 4.800 | 3.000 | 4.500 | 5.000 |
| Total weight | 480.000 | 300.000 | 300.000 | 340.000 |

Composition of aliskiren 150 mg (free base) uncoated tablets in % by weight.

| | Roller | | | |
|---|---|---|---|---|
| | compacted | Dosage | Dosage | Dosage |
| Component | tablet | form 1 | form 2 | form 3 |
| Aliskiren hemi-fumarate | 34.53 | 55.25 | 55.25 | 48.75 |
| Microcrystalline cellulose | 45.97 | 28.25 | 24.08 | 31.545 |
| Polyvinylpyrrolidon K 30 | — | — | 4 | 3.53 |
| Crospovidone | 17.5 | 15 | 14.67 | 14.175 |
| Aerosil 200 | 1 | 0.5 | 0.5 | 0.53 |
| Magnesium stearate | 1 | 1 | 1.5 | 1.47 |
| Total % | 100.00 | 100.00 | 100.00 | 100.00 |

Composition of aliskiren 150 mg (free base) uncoated tablets in mg/unit (divided into inner/outer phase).

| | | Roller | | | |
|---|---|---|---|---|---|
| | | compacted | Dosage | Dosage | Dosage |
| | Component | tablet | form 1 | form 2 | form 3 |
| Inner | Aliskiren hemi-fumarate | 165.75 | 165.75 | 165.75 | 165.75 |
| Phase | Microcrystalline | 220.65 | 84.75 | 72.25 | 90.25 |
| | cellulose | | | | |
| | Polyvinylpyrrolidon K 30 | — | — | 12.00 | 12.00 |
| | Crospovidone | 36.00 | — | — | 14.20 |
| | Aerosil 200 | — | — | — | — |
| | Magnesium stearate | 2.40 | — | — | — |
| Outer | Crospovidone | 48.00 | 45.00 | 44.00 | 34.00 |
| phase | Microcrystalline | — | — | — | 17.00 |
| | cellulose | | | | |
| | Aerosil 200 | 4.80 | 1.50 | 1.50 | 1.80 |
| | Magnesium stearate | 2.40 | 3.00 | 4.50 | 5.00 |
| | Total weight | 480.00 | 300.00 | 300.00 | 340.00 |

Composition of aliskiren 150 mg (free base) uncoated tablets in % by weight (divided into inner/outer phase).

| | | Roller | | | |
|---|---|---|---|---|---|
| | | compacted | Dosage | Dosage | Dosage |
| | Component | tablet | form 1 | form 2 | form 3 |
| Inner | Aliskiren hemi-fumarate | 34.53 | 55.25 | 55.25 | 48.75 |
| Phase | Microcrystalline | 45.97 | 28.25 | 24.08 | 26.545 |
| | cellulose | | | | |
| | Polyvinylpyrrolidon K 30 | — | — | 4 | 3.530 |
| | Crospovidone | 7.5 | — | — | 4.175 |
| | Aerosil 200 | — | — | — | — |
| | Magnesium stearate | 0.5 | — | — | — |
| Outer | Crospovidone | 10 | 15 | 14.67 | 10 |
| phase | Microcrystalline | — | — | — | 5 |
| | cellulose | | | | |
| | Aerosil 200 | 1 | 0.5 | 0.5 | 0.53 |
| | Magnesium stearate | 0.5 | 1 | 1.5 | 1.47 |
| | Total % | 100.00 | 100.00 | 100.00 | 100.00 |

### EXAMPLE 2

Composition of aliskiren (dosage form 3) film-coated tablets in mg/unit.

| | Dosage form 3/Strength | | |
|---|---|---|---|
| | 75 mg | | 300 mg |
| Component | (free base) | 150 mg (free base) | (free base) |
| Aliskiren hemi-fumarate | 82.875 | 165.750 | 331.500 |
| Microcrystalline cellulose | 53.625 | 107.250 | 214.500 |
| Polyvinylpyrrolidon K 30 | 6.000 | 12.000 | 24.000 |
| Crospovidone | 24.100 | 48.200 | 96.400 |
| Aerosil 200 | 0.900 | 1.800 | 3.600 |
| Magnesium stearate | 2.500 | 5.000 | 10.000 |
| Total tablet weight | 170.000 | 340.000 | 680.000 |
| Opadry premix white | 9.946 | 16.711 | 23.9616 |
| Opadry premix red | 0.024 | 0.238 | 1.8382 |
| Opadry premix black | 0.030 | 0.051 | 0.2002 |
| Total fim-coated tablet | 180.000 | 357.000 | 706.000 |
| weight | | | |

The dosages forms 1, 2 and 3 may be prepared, e.g., as follows:
1) mixing the active ingredient and additives and granulating said components with a granulation liquid;
2) drying a resulting granulate;
3) mixing the dried granulate with outer phase excipients;
4) compressing a resulting mixture to form a solid oral dosage as a core tablet; and
5) optionally coating a resulting core tablet to give a film-coated tablet.

The granulation liquid can be ethanol, a mixture of ethanol and water, a mixture of ethanol, water and isopropanol, or a solution of polyvinylpyrrolidones (PVP) in the before mentioned mixtures. A preferred mixture of ethanol and water ranges from about 50/50 to about 99/1 (% w/w), most preferrably it is about 94/6 (% w/w). A preferred mixture of ethanol, water and isopropanol ranges from about 45/45/5 to about 98/1/1 (% w/w/w), most preferably from about 88.5/5.5/6.0 to about 91.5/4.5/4.0 (% w/w/w). A preferred concentration of PVP in the above named mixtures ranges from about 5 to about 30% by weight, preferably from about 15 to about 25%, more preferably from about 16 to about 22%.

Attention is drawn to the numerous known methods of granulating, drying and mixing employed in the art, e.g., spray granulation in a fluidized bed, wet granulation in a high-shear mixer, melt granulation, drying in a fluidized-bed dryer, mixing in a free-fall or tumble blender, compressing into tablets on a single-punch or rotary tablet press.

The manufacturing of the granulate can be performed on standard equipment suitable for organic granulation processes. The manufacturing of the final blend and the compression of tablets can also be performed on standard equipment.

For example, step (1) may be carried out by a high-shear granulator, e.g., Collette Gral; step (2) may be conducted in a fluid-bed dryer; step (3) may be carried out by a free-fall mixer (e.g. container blender, tumble blender); and step (4) may be carried out using a dry compression method, e.g., a rotary tablet press.

1. A combination comprising a renin inhibitor, or a pharmaceutically acceptable salt thereof, and at least one therapeutic agent selected from the group consisting of
(a) an insulin secretion enhancer, or a pharmaceutically acceptable salt thereof; and
(b) an insulin sensitizer, or a pharmaceutically acceptable salt thereof.

2. A combination according to claim 1, wherein an insulin secretion enhancer is a glucokinase (GK) activator and an insulin sensitizer is selected from the group consisting of an inhibitor of stearoyl-CoA desaturase-1 (SCD-1), an inhibitor of diacylglycerol acyltransferase 1 (DGAT1), an inhibitor of diacylglycerol acyltransferase 2 (DGAT2) and an inhibitor of phosphotyrosine phosphatase (PTPase).

3. A combination according to claim 2, wherein a renin inhibitor is selected from the group consisting of RO 66-1132, RO 66-1168 and a compound of the formula 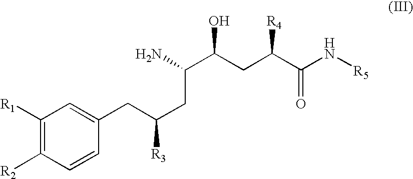
wherein R₁ is halogen, C₁₋₆halogenalkyl, C₁₋₆alkoxy-C₁₋₆alkyloxy or C₁₋₆alkoxy-C₁₋₆alkyl; R₂ is halogen, C₁₋₄alkyl or C₁₋₄alkoxy; R₃ and R₄ are independently branched C₃₋₆alkyl; and R₅ is cycloalkyl, C₁₋₆alkyl, C₁₋₆hydroxyalkyl, C₁₋₆alkoxy-C₁₋₆alkyl, C₁₋₆alkanoyloxy-C₁₋₆alkyl, C₁₋₆aminoalkyl, C₁₋₆alkylamino-C₁₋₆alkyl, C₁₋₆dialkylamino-C₁₋₆alkyl, C₁₋₆alkanoylamino-C₁₋₆alkyl, HO(O)C—C₁₋₆alkyl, C₁₋₆alkyl-O—(O)C—C₁₋₆alkyl, H₂N—C(O)—C₁₋₆alkyl, C₁₋₆alkyl-HN—C(O)—C₁₋₆alkyl or (C₁₋₆alkyl)₂N—C(O)—C₁₋₆alkyl; or a pharmaceutically acceptable salt thereof.

4. A combination according to claim 3, wherein a renin inhibitor is a compound of formula (III) having the formula 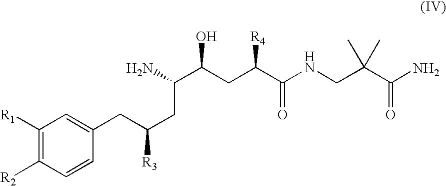
wherein R₁ is 3-methoxypropyloxy; R₂ is methoxy; and R₃ and R₄ are isopropyl; or a pharmaceutically acceptable salt thereof.

5. A combination according to claim 4, wherein the compound of formula (IV) is in the form of the hemi-fumarate salt thereof.

6. A combination according to claim 2 wherein a glucokinase activator is selected from the group consisting of 6-[(3-isobutoxy-5-isopropoxybenzoyl)amino]nicotinic acid (GKA1), 5-({3-isopropoxy-5-[2-(3-thienyl)ethoxy]benzoyl}amino)-1,3,4-thiadiazole-2-carboxylic acid (GKA2), 2-(S)-Cyclohexyl-1-(R)-(4-methanesulfonyl-phenyl)-cyclopropanecarboxylic acid thiazol-2-ylamide (LY2121260) and RO-28-1675 or a pharmaceutically acceptable salt thereof.

7. A combination according to claim 2, wherein a glucokinase activator is a compound of the formula
R—NH-Q (IX)
wherein
(i) Q is a 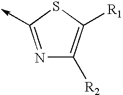
radical in which R₁ and R₂ are independently hydrogen or halogen; or
Q is a 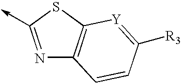
radical in which R₃ is hydrogen, halogen, alkyl, cycloalkyl, aryl, alkoxy, cycloalkoxy, aryloxy, alkylthio, cycloalkylthio, arylthio, acyl, sulfonyl, alkylamino, cycloalkylamino, arylamino, acylamino, sulfonamide or alkoxycarbonyl; Y is CH or nitrogen; and
R is a radical of the formula 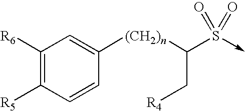
wherein
R₄ is C₂₋₄alkyl, C₃₋₇cycloalkyl or C₅₋₇heterocycloalkyl;
R₅ and R₆ are independently hydrogen, halogen, cyano, R₇, —C(O)R₇ or —S(O)₂R₇ wherein
R₇ is —(CR₈R₉)ₘ—W—R₁₀ in which
R₈ and R₉ are independently hydrogen or lower alkyl;
W is a bond, O, S or —NR₁₁ in which
R₁₁ is hydrogen or lower alkyl;
R₁₀ is hydrogen, alkyl, cycloalkyl, aryl or heterocyclyl; or R₁₀ and R₁₁, combined, are alkylene which together with the nitrogen atom to which they are attached form a 5- to 7-membered ring;
m is zero or an integer from 1 to 5;
n is zero or an integer of 1 or 2;
or an optical isomer thereof; or a pharmaceutically acceptable salt thereof; or
(ii) Q is a 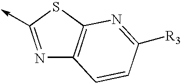
radical in which R₃ is hydrogen, halogen, alkyl, cycloalkyl, aryl, alkoxy, cycloalkoxy, aryloxy, alkylthio, cycloalkylthio, arylthio, acyl, sulfonyl, alkylamino, cycloalkylamino, arylamino, acylamino, sulfonamido or alkoxycarbonyl; and
R is a radical of the formula 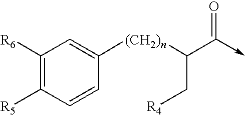
wherein
R₄ is C₂₋₄alkyl, C₃₋₇cycloalkyl or C₅₋₇heterocycloalkyl;
R₅ and R₆ are independently hydrogen, halogen, cyano, R₇, —C(O)R₇ or —S(O)₂R₇ wherein
R₇ is —(CR₈R₉)ₘ—W—R₁₀ in which
R₈ and R₉ are independently hydrogen or lower alkyl;
W is a bond, O, S or —NR₁₁ in which
R₁₁ is hydrogen or lower alkyl;
R₁₀ is hydrogen, alkyl, cycloalkyl, aryl or heterocyclyl; or R₁₀ and R₁₁, combined, are alkylene which together with the nitrogen atom to which they are attached form a 5- to 7-membered ring;
m is zero or an integer from 1 to 5;
n is zero or an integer of 1 or 2;
or an optical isomer thereof; or a pharmaceutically acceptable salt thereof; or
(iii) Q is a 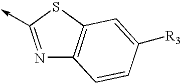
radical in which R₃ is hydrogen, halogen, alkyl, cycloalkyl, aryl, alkoxy, cycloalkoxy, aryloxy, alkylthio, cycloalkylthio, arylthio, acyl, sulfonyl, alkylamino, cycloalkylamino, arylamino, acylamino, sulfonamido or alkoxycarbonyl; and
R is a radical of the formula 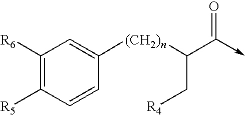
wherein
R₄ is C₂₋₄alkyl, C₃₋₇cycloalkyl or C₅₋₇heterocycloalkyl;
R₅ and R₆ are independently hydrogen, halogen, cyano, R₇, —C(O)R₇ or —S(O)₂R₇ wherein
R₇ is —(CR₈R₉)ₘ—W—R₁₀ in which
R₈ and R₉ are independently hydrogen or lower alkyl;
W is a bond, O, S or —NR₁₁ in which
R₁₁ is hydrogen or lower alkyl;
R₁₀ is hydrogen, alkyl, cycloalkyl, aryl or heterocyclyl; or R₁₀ and R₁₁, combined, are alkylene which together with the nitrogen atom to which they are attached form a 5- to 7-membered ring;
m is zero or an integer from 1 to 5;
n is zero or an integer of 1 or 2;
or an optical isomer thereof; or a pharmaceutically acceptable salt thereof; or
(iv) Q is a 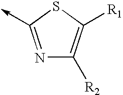
radical, wherein R₁ and R₂ are independently hydrogen or halogen; and
R is a radical of the formula 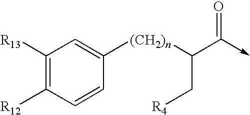
wherein
R₄ is C₂₋₄alkyl, C₃₋₇cycloalkyl or C₅₋₇heterocycloalkyl;
R₁₂ and R₁₃ are independently hydrogen, halogen, cyano, R₁₄, —C(O)R₁₄, or —S(O)₂R₁₄ wherein
R₁₄ is —(CR₈R₉)ₘ—W—R₁₅ in which
R₈ and R₉ are independently hydrogen or lower alkyl;
W is a bond, O, S or —NR₁₁ in which
R₁₁ is hydrogen or lower alkyl;
R₁₅ is cycloalkyl, aryl or heterocyclyl; or R₁₅ and R₁₁, combined, are alkylene which together with the nitrogen atom to which they are attached form a 5- to 7-membered ring;
m is zero or an integer from 1 to 5;
n is zero or an integer of 1 or 2;
or an optical isomer thereof; or a pharmaceutically acceptable salt thereof.

8. A combination according to claim 2, wherein an inhibitor of stearoyl-CoA desaturase (SCD-1) is selected from the group consisting of
1-Pentyl-3-{6-[4-(2-trifluoromethylbenzoyl)piperazin-1-yl]pyridazin-3-yl}urea;
1-Benzyl-3-{6-[4-(2-trifluoromethylbenzoyl)piperazin-1-yl]pyridazin-3-yl}urea;
1-(4-Fluorophenyl)-3-{6-[4-(2-trifluoromethylbenzoyl)piperazin-1-yl]-pyridazin-3-yl}urea;
1-(2-Fluorophenyl)-3-{6-[4-(2-trifluoromethylbenzoyl)piperazin-1-yl]pyridazin-3-yl}urea;
1-[1-(4-Fluorophenyl)ethyl]-3-{6-[4-(2-trifluoromethylbenzoyl)-piperazin-1-yl]pyridazin-3-yl}urea;
1-[1-(4-Fluorophenyl)ethyl]-3-{6-[4-(2-trifluoromethylbenzoyl)piperazin-1-yl]pyridazin-3-yl}urea;
1-[3-(4-Fluorophenyl)propyl]-3-{6-[4-(2-trifluoromethylbenzoyl)piperazin-1-yl]pyridazin-3-yl}urea;
1-Phenethyl-3-{6-[4-(2-trifluoromethylbenzoyl)piperazin-1-yl]-pyridazin-3-yl}urea;
1-(4-Fluorobenzyl)-3-{6-[4-(2-trifluoromethylbenzoyl)piperazin-1-yl]-pyridazin-3-yl}urea;
1-(3,4-Dichlorobenzyl)-3-{6-[4-(2-trifluoromethylbenzoyl)piperazin-1-yl]-pyridazin-3-yl}urea;
1-(2-Phenylcyclopropyl)-3-{6-[4-(2-trifluoromethylbenzoyl)piperazin-1-yl]-pyridazin-3-yl}urea;
1-Cyclopentyl-3-{6-[4-(2-trifluoromethylbenzoyl)piperazin-1-yl]-pyridazin-3-yl}urea;
1-(3-Cyclopropylpropyl)-3-{6-[4-(2-trifluoromethylbenzoyl)piperazin-1-yl]-pyridazin-3-yl}urea;
1-Cyclopropylmethyl-3-{6-[4-(2-trifluoromethylbenzoyl)piperazin-1-yl]-pyridazin-3-yl}urea;
1-(2-Cyclopropylethyl)-3-{6-[4-(2-trifluoromethylbenzoyl)-piperazin-1-yl]-pyridazin-3-yl}urea;
1-(2-Cyclopropylethyl)-3-{6-[4-(2-fluoro-6-trifluoromethyl benzoyl)piperazin-1-yl]-pyridazin-3-yl}urea;
1-(2-Cyclopropylethyl)-3-{6-[4-(5-fluoro-2-trifluoromethylbenzoyl)piperazin-1-yl]-pyridazin-3-yl}urea;
1-Cyclohexyl-3-{6-[4-(2-trifluoromethylbenzoy-l)piperazin-1-yl]-pyridazin-3-yl}urea;
1-(2-Cyclopropylethyl)-3-{6-[4-(2,6-difluorobenzoyl)piperazin-1-yl]-pyridazin-3-yl}urea;
1-(3-Cyclopropylpropyl)-3-{6-[4-(5-fluoro-2-trifluoromethylbenzoyl)piperazin-1-yl]-pyridazin-3-yl}urea;
4-Phenyl-N-{6-[4-(2-trifluoromethylbenzoyl)piperazin-1-yl]-pyridazin-3-yl}butyramide;
4-Methylpentanoic acid {6-[4-(2-trifluoromethylbenzoyl)-piperazin-1-yl]-pyridazin-3-yl}amide;
3-Cyclopentyl-N-{6-[4-(2-trifluoromethylbenzoyl)piperazin-1-yl]-pyridazin-3-yl}propionamide;
6-[4-(2-Trifluoromethylbenzoyl)piperazin-1-yl]-pyridazine-3-carboxylic acid phenethylamide;
6-[4-(2-Trifluoromethylbenzoyl)piperazin-1-yl]-pyridazine-3-carboxylic acid [2-(4-methoxyphenyl)ethyl]amide;
6-[4-(2-Trifluoromethylbenzoyl)piperazin-1-yl]-pyridazine-3-carboxylic acid [2-(3-fluorophenyl)ethyl]amide;
6-[4-(2-Trifluoromethylbenzoyl)piperazin-1-yl]-pyridazine-3-carboxylic acid (3-phenylpropyl)amide;
6-[4-(2-Trifluoromethylbenzoyl)piperazin-1-yl]-pyridazine-3-carboxylic acid [2-(4-fluorophenyl)ethyl]amide;
4-Methyl-2-({6-[4-(2-trifluoromethylbenzoyl)piperazin-1-yl]-pyridazine-3-carbonyl}amino)pentanoic acid methyl ester;
6-[4-(2-Trifluoromethylbenzoyl)piperazin-1-yl]-pyridazine-3-carboxylic acid (3-methylbutyl)amide;
6-[4-(5-Fluoro-2-trifluoromethylbenzoyl)piperazin-1-yl]-pyridazine-3-carboxylic acid (3-methylbutyl)amide;
6-[4-(4-Fluoro-2-trifluoromethylbenzoyl)piperazin-1-yl]-pyridazine-3-carboxylic acid (3-methylbutyl)amide;
4-Methyl-2-({6-[4-(2-trifluoromethyl benzoyl)piperazin-1-yl]-pyridazine-3-carbonyl}amino)pentanoic acid;
6-[4-(2-Trifluoromethylbenzoyl)piperazin-1-yl]-pyridazine-3-carboxylic acid (2-cyclopropylethyl)amide;
6-[4-(5-Fluoro-2-trifluoromethylbenzoyl)piperazin-1-yl]-pyridazine-3-carboxylic acid (2-cyclopropylethyl)amide;
6-[4-(2-Trifluoromethylbenzoyl)piperazin-1-yl]-pyridazine-3-carboxylic acid cyclopropylmethylamide;
4-(4-Methoxyphenyl)-N-{6-[4-(2-trifluoromethylbenzoyl)-piperazin-1-yl]-pyridazin-3-yl}butyramide;
3-(4-Fluorophenyl)-N-{6-[4-(2-trifluoromethylbenzoyl)-piperazin-1-yl]-pyridazin-3-yl}propionamide;
4-Cyclohexyl-N-{6-[4-(2-trifluoromethylbenzoyl)piperazin-1-yl]-pyridazin-3-yl}butyramide;
2,2,3,3-Tetramethylcyclopropanecarboxylic acid {6-[4-(2-trifluoromethylbenzoyl)piperazin-1-yl]pyridazin-3-yl}amide;
Cyclopropanecarboxylic acid {6-[4-(2-trifluoromethylbenzoyl)piperazin-1-yl]-pyridazin-3-yl}amide;
1-Trifluoromethylcyclopropanecarboxylic acid {6-[4-(2-trifluoromethylbenzoyl)piperazin-1-yl]-pyridazin-3-yl}amide;
2-Phenylcyclopropanecarboxylic acid {6-[4-(2-trifluoromethylbenzoyl)piperazin-1-yl]-pyridazin-3-yl}amide;
6-[4-(2-Trifluoromethylbenzoyl)piperazin-1-yl]-pyridazine-3-carboxylic acid indan-1-ylamide;
6-[4-(2-Trifluoromethylbenzoyl)piperazin-1-yl]-pyridazine-3-carboxylic acid (2-oxo-1,3-diaza-bicyclo[3.1.0]hex-3-en-4-yl)amide;
6-[4-(2-Trifluoromethylbenzoyl)piperazin-1-yl]-pyridazine-3-carboxylic acid (5-oxo-4,5-dihydro-1H-pyrazol-3-yl)amide;
6-[4-(2-Trifluoromethylbenzoyl)piperazin-1-yl]-pyridazine-3-carboxylic acid indan-5-ylamide;
5-[1,2]Dithiolan-3-yl-pentanoic acid {6-[4-(2-trifluoromethyl-benzoyl)-piperazin-1-yl]-pyridazin-3-yl}amide;
6-[4-(2-Trifluoromethyl-benzoyl)-piperazin-1-yl]-pyridazine-3-carboxylic acid (2-thiophen-2-yl-ethyl)amide;
6-[4-(2-Trifluoromethylbenzoyl)piperazin-1-yl]-pyridazine-3-carboxylic acid (2-benzo[1,3]dioxol-5-yl-ethyl)amide;
6-[4-(2-Trifluoromethylbenzoyl)piperazin-1-yl]-pyridazine-3-carboxylic acid (2,2-difluoro-2-pyridin-2-ylethyl)amide;
6-[4-(2-Trifluoromethylbenzoyl)piperazin-1-yl]-pyridazine-3-carboxylic acid (2-pyridin-2-ylethyl)amide;
6-[4-(2-Trifluoromethyl benzoyl)piperazin-1-yl]-pyridazine-3-carboxylic acid (pyridin-2-yl-methyl)amide;
6-[4-(2-Trifluoromethylbenzoyl)piperazin-1-yl]-pyridazine-3-carboxylic acid (5-chloropyridin-2-yl)amide;
6-[4-(2-Trifluoromethylbenzoyl)piperazin-1-yl]-pyridazine-3-carboxylic acid (5-trifluoromethylpyridin-2-yl)-amide;
6-[4-(2-Trifluoromethylbenzoyl)piperazin-1-yl]-pyridazine-3-carboxylic acid (7H-purin-6-yl)amide;
6-[4-(2-Trifluoromethylbenzoyl)piperazin-1-yl]-pyridazine-3-carboxylic acid pyrazin-2-ylamide;
6-[4-(2-Trifluoromethylbenzoyl)piperazin-1-yl]-pyridazine-3-carboxylic acid (1H-tetrazol-5-yl)amide;
6-[4-(2-Trifluoromethylbenzoyl)piperazin-1-yl]-pyridazine-3-carboxylic acid (2H-[1,2,4]triazol-3-yl)amide;
6-[4-(2-Trifluoromethylbenzoyl)piperazin-1-yl]-pyridazine-3-carboxylic acid (3-methylisoxazol-5-yl)amide;
6-[4-(2-Trifluoromethylbenzoyl)piperazin-1-yl]-pyridazine-3-carboxylic acid (5-methylisoxazol-3-yl)amide;
6-[4-(2-Trifluoromethyl benzoyl)piperazin-1-yl]-pyridazine-3-carboxylic acid (1H-pyrazol-3-yl)amide;
6-[4-(2-Trifluoromethylbenzoyl)piperazin-1-yl]-pyridazine-3-carboxylic acid (5-methyl-1H-pyrazol-3-yl)amide;
6-[4-(2-Trifluoromethylbenzoyl)piperazin-1-yl]-pyridazine-3-carboxylic acid pyrimidin-2-ylamide;
6-[4-(2-Trifluoromethylbenzoyl)piperazin-1-yl]-pyridazine-3-carboxylic acid pyrazin-2-ylamide;
6-[4-(2-Trifluoromethylbenzoyl)piperazin-1-yl]-pyridazine-3-carboxylic acid (4-methylpyrimidin-2-yl)amide;
6-[4-(2-Trifluoromethylbenzoyl)piperazin-1-yl]-pyridazine-3-carboxylic acid (2-oxo-2,3-dihydro-pyrimidin-4-yl)amide;
6-[4-(2-Trifluoromethylbenzoyl)piperazin-1-yl]-pyridazine-3-carboxylic acid (6-oxo-1,6-dihydro-pyrimidin-2-yl)amide;
6-[4-(2-Trifluoromethylbenzoyl)piperazin-1-yl]-pyridazine-3-carboxylic acid [1,3,4]thiadiazol-2-ylamide;
6-[4-(2-Trifluoromethylbenzoyl)piperazin-1-yl]-pyridazine-3-carboxylic acid thiazol-2-ylamide;
6-[4-(2-Trifluoromethylbenzoyl)piperazin-1-yl]-pyridazine-3-carboxylic acid pyridin-2-ylamide;
6-[4-(2-Trifluoromethylbenzoyl)piperazin-1-yl]-pyridazine-3-carboxylic acid pyridazin-3-ylamide;
6-[4-(2-Trifluoromethylbenzoyl)piperazin-1-yl]-pyridazine-3-carboxylic acid pyridin-3-ylamide;
6-[4-(2-Trifluoromethylbenzoyl)piperazin-1-yl]-pyridazine-3-carboxylic acid pyridin-4-ylamide;
6-[4-(2-Trifluoromethylbenzoy-l)piperazin-1-yl]-pyridazine-3-carboxylic acid (6-oxo-1,6-dihydro-[1,3,5]triazin-2-yl)amide;
6-[4-(2-Trifluoromethylbenzoyl)piperazin-1-yl]-pyridazine-3-carboxylic acid (5-fluoropyridin-2-yl)amide;
6-[4-(2-Trifluoromethylbenzoyl)-piperazin-1-yl]-pyridazine-3-carboxylic acid (5-cyanopyridin-2-yl)amide;
6-[4-(2-Trifluoromethylbenzoyl)-piperazin-1-yl]-pyridazine-3-carboxylic acid (4,6-dimethyl-pyrimidin-2-yl)amide;
6-[4-(2-Trifluoromethylbenzoyl)-piperazin-1-yl]-pyridazine-3-carboxylic acid (2-chloropyridin-4-yl)amide;
6-[4-(2-Trifluoromethylbenzoyl)-piperazin-1-yl]-pyridazine-3-carboxylic acid (1H-indol-6-yl)amide;
6-[4-(2-Trifluoromethylbenzoyl)-piperazin-1-yl]-pyridazine-3-carboxylic acid (1H-indol-4-yl)amide;
6-[4-(2-Trifluoromethylbenzoyl)-piperazin-1-yl]-pyridazine-3-carboxylic acid (1H-indazol-5-yl)amide;
6-[4-(2-Trifluoromethylbenzoyl)-piperazin-1-yl]-pyridazine-3-carboxylic acid (1H-indazol-6-yl)amide;
6-[4-(2-Trifluoromethylbenzoyl)-piperazin-1-yl]-pyridazine-3-carboxylic acid (4-methylthiazol-2-yl)amide;
6-[4-(2-Trifluoromethylbenzoyl)-piperazin-1-yl]-pyridazine-3-carboxylic acid (5-methylthiazol-2-yl)amide;
6-[4-(2-Trifluoromethylbenzoyl)-piperazin-1-yl]-pyridazine-3-carboxylic acid (5-thioxo-4,5-dihydro-1H-[1,2,4]triazol-3-yl)amide;
6-[4-(2-Trifluoromethylbenzoyl)piperazin-1-yl]-pyridazine-3-carboxylic acid (1H-benzoimidazol-2-yl)amide;
6-[4-(2-Trifluoromethylbenzoyl)piperazin-1-yl]-pyridazine-3-carboxylic acid (6-methylpyridazin-3-yl)amide;
6-[4-(2-Trifluoromethylbenzoyl)piperazin-1-yl]-pyridazine-3-carboxylic acid (6-methoxypyridazin-3-yl)amide;
6-[4-(2-Trifluoromethylbenzoyl)-piperazin-1-yl]-pyridazine-3-carboxylic acid (6-chloropyridazin-3-yl)amide;
6-[4-(2-Trifluoromethylbenzoyl)piperazin-1-yl]-pyridazine-3-carboxylic acid (4-chlorophenyl)amide;
6-[4-(2-Trifluoromethylbenzoyl)piperazin-1-yl]-pyridazine-3-carboxylic acid (4-carbamoyl-phenyl)amide;
6-[4-(2-Trifluoromethylbenzoyl)piperazin-1-yl]-pyridazine-3-carboxylic acid (3-carbamoyl-phenyl)amide;
6-[4-(2-Trifluoromethylbenzoyl)piperazin-1-yl]-pyridazine-3-carboxylic acid m-tolylamide;
6-[4-(2-Trifluoromethylbenzoyl)piperazin-1-yl]-pyridazine-3-carboxylic acid p-tolylamide;
6-[4-(2-Trifluoromethylbenzoyl)piperazin-1-yl]-pyridazine-3-carboxylic acid o-tolylamide;
6-[4-(2-Trifluoromethylbenzoyl)piperazin-1-yl]-pyridazine-3-carboxylic acid (2-propylphenyl)amide;
6-[4-(2-Trifluoromethylbenzoyl)piperazin-1-yl]-pyridazine-3-carboxylic acid (4-propylphenyl)amide;
6-[4-(2-Trifluoromethylbenzoyl)piperazin-1-yl]-pyridazine-3-carboxylic acid (4-isopropylphenyl)amide;
6-[4-(2-Trifluoromethylbenzoyl)piperazin-1-yl]-pyridazine-3-carboxylic acid (2-isopropylphenyl)amide;
6-[4-(2-Trifluoromethylbenzoyl)piperazin-1-yl]-pyridazine-3-carboxylic acid (2-chlorophenyl)amide;
6-[4-(2-Trifluoromethylbenzoyl)piperazin-1-yl]-pyridazine-3-carboxylic acid (2-cyano-3-fluorophenyl)amide;
6-[4-(2-Trifluoromethylbenzoyl)piperazin-1-yl]-pyridazine-3-carboxylic acid (2,4-dimethylphenyl)amide;
6-[4-(2-Trifluoromethylbenzoyl)piperazin-1-yl]-pyridazine-3-carboxylic acid (2,5-dimethylphenyl)amide;
6-[4-(2-Trifluoromethylbenzoyl)piperazin-1-yl]-pyridazine-3-carboxylic acid (2,6-dimethylphenyl)amide;
6-[4-(2-Trifluoromethylbenzoyl)piperazin-1-yl]-pyridazine-3-carboxylic acid (2,3-dimethylphenyl)amide;
6-[4-(2-Trifluoromethylbenzoyl)piperazin-1-yl]-pyridazine-3-carboxylic acid (3,5-dimethylphenyl)amide;
6-[4-(2-Trifluoromethylbenzoyl)piperazin-1-yl]-pyridazine-3-carboxylic acid (3,4-dimethylphenyl)amide;
6-[4-(2-Trifluoromethylbenzoyl)piperazin-1-yl]-pyridazine-3-carboxylic acid (4-ethylphenyl)amide;
6-[4-(2-Trifluoromethylbenzoyl)piperazin-1-yl]-pyridazine-3-carboxylic acid (2-ethylphenyl)amide;
6-[4-(2-Trifluoromethylbenzoyl)piperazin-1-yl]-pyridazine-3-carboxylic acid (3-fluoro-2-methylphenyl)amide;
6-[4-(2-Trifluoromethylbenzoyl)piperazin-1-yl]-pyridazine-3-carboxylic acid (2-fluoro-4-methylphenyl)amide;
6-[4-(2-Trifluoromethylbenzoyl)piperazin-1-yl]-pyridazine-3-carboxylic acid (4-fluoro-2-methylphenyl)amide;
6-[4-(2-Trifluoromethylbenzoyl)piperazin-1-yl]-pyridazine-3-carboxylic acid (2-fluoro-5-methylphenyl)amide;
6-[4-(2-Trifluoromethylbenzoyl)piperazin-1-yl]-pyridazine-3-carboxylic acid (3-fluoro-5-methylphenyl)amide;
6-[4-(2-Trifluoromethyl benzoyl)piperazin-1-yl]-pyridazine-3-carboxylic acid (3-fluorophenyl)amide;
6-[4-(2-Trifluoromethylbenzoyl)piperazin-1-yl]-pyridazine-3-carboxylic acid (2-fluorophenyl)amide;
6-[4-(2-Trifluoromethylbenzoyl)piperazin-1-yl]-pyridazine-3-carboxylic acid (4-fluorophenyl)amide;
6-[4-(2-Trifluoromethylbenzoyl)piperazin-1-yl-pyridazine-3-carboxylic acid (2,4-difluorophenyl)amide;
6-[4-(2-Trifluoromethylbenzoyl)piperazin-1-yl]-pyridazine-3-carboxylic acid (2,5-difluorophenyl)amide;
6-[4-(2-Trifluoromethylbenzoyl)piperazin-1-yl]-pyridazine-3-carboxylic acid (3,4-difluorophenyl)amide;
6-[4-(2-Trifluoromethylbenzoyl)piperazin-1-yl]-pyridazine-3-carboxylic acid (2,3-difluorophenyl)amide;
6-[4-(2-Trifluoromethylbenzoyl)piperazin-1-yl]-pyridazine-3-carboxylic acid (2,6-difluorophenyl)amide;
6-[4-(2-Trifluoromethylbenzoyl)piperazin-1-yl]-pyridazine-3-carboxylic acid (4-cyanophenyl)amide;
6-[4-(2-Trifluoromethylbenzoyl)piperazin-1-yl]-pyridazine-3-carboxylic acid (2-cyanophenyl)amide;
6-[4-(2-Trifluoromethylbenzoyl)piperazin-1-yl]-pyridazine-3-carboxylic acid (3-cyanophenyl)amide;
6-[4-(2-Trifluoromethylbenzoyl)piperazin-1-yl]-pyridazine-3-carboxylic acid (3-chlorophenyl)amide;
6-[4-(2-Trifluoromethylbenzoyl)piperazin-1-yl]-pyridazine-3-carboxylic acid (3-chloro-2-methylphenyl)amide;
6-[4-(2-Trifluoromethylbenzoyl)piperazin-1-yl]-pyridazine-3-carboxylic acid (2-chloro-3-methylphenyl)amide;
6-[4-(2-Trifluoromethylbenzoyl)piperazin-1-yl]-pyridazine-3-carboxylic acid (2,5-dichlorophenyl)amide;
6-[4-(2-Trifluoromethylbenzoyl)piperazin-1-yl]-pyridazine-3-carboxylic acid (2-chloro-5-methylphenyl)amide;
6-[4-(2-Trifluoromethyl benzoyl)piperazin-1-yl]-pyridazine-3-carboxylic acid (2-chloro-6-methylphenyl)amide;
6-[4-(2-Trifluoromethylbenzoyl)piperazin-1-yl]-pyridazine-3-carboxylic acid (4-chloro-2-methylphenyl)amide;
6-[4-(2-Trifluoromethylbenzoyl)piperazin-1-yl]-pyridazine-3-carboxylic acid (4-chloro-3-methylphenyl)amide;
6-[4-(2-Trifluoromethylbenzoyl)piperazin-1-yl]-pyridazine-3-carboxylic acid (3-chloro-4-methylphenyl)amide;
6-[4-(2-Trifluoromethylbenzoyl)piperazin-1-yl]-pyridazine-3-carboxylic acid (2-chloro-4-methylphenyl)amide;
6-[4-(2-Trifluoromethylbenzoyl)piperazin-1-yl]-pyridazine-3-carboxylic acid (2-chloro-5-fluorophenyl)amide;
6-[4-(2-Trifluoromethylbenzoyl)piperazin-1-yl]-pyridazine-3-carboxylic acid (5-chloro-2-fluorophenyl)amide;
6-[4-(2-Trifluoromethylbenzoyl)piperazin-1-yl]-pyridazine-3-carboxylic acid (2,5-difluorophenyl)amide;
6-[4-(2-Trifluoromethylbenzoyl)piperazin-1-yl]-pyridazine-3-carboxylic acid (2,6-dichlorophenyl)amide;
6-[4-(2-Trifluoromethylbenzoyl)piperazin-1-yl]-pyridazine-3-carboxylic acid (2-trifluoromethylphenyl)amide;
6-[4-(2-Trifluoromethylbenzoyl)piperazin-1-yl]-pyridazine-3-carboxylic acid (4-trifluoromethylphenyl)amide;
6-[4-(2-Trifluoromethylbenzoyl)-piperazin-1-yl]-pyridazine-3-carboxylic acid (3-trifluoromethylphenyl)amide;
6-[4-(2-Trifluoromethylbenzoyl)piperazin-1-yl]-pyridazine-3-carboxylic acid phenylamide;
6-[4-(2-Trifluoromethylbenzoyl)piperazin-1-yl]-pyridazine-3-carboxylic acid (5-chloro-2-methoxyphenyl)amide;
6-[4-(2-Trifluoromethylbenzoyl)piperazin-1-yl]-pyridazine-3-carboxylic acid (2,5-dimethoxyphenyl)amide;
6-[4-(2-Trifluoromethylbenzoyl)piperazin-1-yl]-pyridazine-3-carboxylic acid (2-chloro-4-methoxyphenyl)amide;
6-[4-(2-Trifluoromethylbenzoyl)-piperazin-1-yl]-pyridazine-3-carboxylic acid (4-methoxyphenyl)amide;
6-[4-(2-Trifluoromethylbenzoyl)piperazin-1-yl]-pyridazine-3-carboxylic acid (2-methoxyphenyl)amide;
6-[4-(2-Trifluoromethylbenzoyl)piperazin-1-yl]-pyridazine-3-carboxylic acid (3-methoxyphenyl)amide;
4-({6-[4-(2-Trifluoromethylbenzoyl)piperazin-1-yl]-pyridazine-3-carbonyl}amino)-benzoic acid methyl ester;
4-({6-[4-(2-Trifluoromethylbenzoyl)piperazin-1-yl]-pyridazine-3-carbonyl}amino)-benzoic acid;
2-({6-[4-(2-Trifluoromethylbenzoyl)piperazin-1-yl]-pyridazine-3-carbonyl}amino)-benzoic acid methyl ester;
2-({6-[4-(2-Trifluoromethylbenzoyl)piperazin-1-yl]-pyridazine-3-carbonyl}amino)-benzoic acid;
6-[4-(2-Trifluoromethylbenzoyl)piperazin-1-yl]-pyridazine-3-carboxylic acid (3,4-dichlorophenyl)amide;
6-[4-(2-Trifluoromethylbenzoyl)piperazin-1-yl]-pyridazine-3-carboxylic acid [2-(2,4-fluorophenyl)ethyl]amide;
6-[4-(2-Trifluoromethylbenz-oyl)piperazin-1-yl]-pyridazine-3-carboxylic acid [2-(2-fluorophenyl)ethyl]amide;
6-[4-(2-Trifluoromethylbenzoyl)piperazin-1-yl]-pyridazine-3-carboxyli-c acid [2-(4-chlorophenyl)ethyl]amide;
6-[4-(2-Trifluoromethylbenzoyl)piperazin-1-yl]-pyridazine-3-carboxylic acid [2-(3-chlorophenyl)ethyl]amide;
6-[4-(2-Trifluoromethylbenzoyl)piperazin-1-yl]-pyridazine-3-carboxylic acid (2-phenylpropyl)amide;
6-[4-(2-Trifluoromethylbenzoyl)piperazin-1-yl]-pyridazine-3-carboxylic acid (2-biphenyl-4-ylethyl)amide;
(R)-6-[4-(2-Trifluoromethylbenzoyl)piperazin-1-yl]-pyridazine-3-carboxylic acid (2-hydroxy-2-phenylethyl)amide;
(S)-6-[4-(2-Trifluoromethylbenzoyl)-piperazin-1-yl]-pyridazine-3-carboxylic acid (2-hydroxy-2-phenylethyl)amide;
Acetic acid 1-phenyl-2-({6-[4-(2-trifluoromethyl-benzoyl)-piperazin-1-yl]-pyridazine-3-carbonyl}amino)ethyl ester;
6-[4-(2-Trifluoromethylbenzoyl)piperazin-1-yl]-pyridazine-3-carboxylic acid [3-(4-fluorophenyl)propyl]amide;
6-[4-(2-Trifluoromethylbenzoyl)piperazin-1-yl]-pyridazine-3-carboxylic acid (2,2-difluoro-2-phenylethyl)amide;
6-[4-(2-Trifluoromethylbenzoyl)piperazin-1-yl]-pyridazine-3-carboxylic acid [2-(3-fluorophenyl)-2-hydroxyethyl]amide;
6-[4-(2-Trifluoromethylbenzoyl)piperazin-1-yl]-pyridazine-3-carboxylic acid (4-hydroxybutyl)amide;
6-[4-(2-Trifluoromethylbenzoyl)piperazin-1-yl]-pyridazine-3-carboxylic acid (3-hydroxy-4,4-dimethylpentyl)amide;
6-[4-(2-Trifluoromethylbenzoyl)piperazin-1-yl]-pyridazine-3-carboxylic acid (3-hydroxy-3-methylbutyl)amide;
6-[4-(2-Trifluoromethylbenzoyl)piperazin-1-yl]-pyridazine-3-carboxylic acid (2-hydroxy-3,3-dimethylbutyl)amide;
6-[4-(5-Fluoro-2-trifluoromethylbenzoyl)piperazin-1-yl]-pyridazine-3-carboxylic acid (2-hydroxy-3,3-dimethylbutyl)amide;
6-[4-(2-Nitrobenzoyl)piperazin-1-yl]-pyridazine-3-carboxylic acid (3-methylbutyl)amide;
6-[4-(2-Chlorobenzoyl)piperazin-1-yl]-pyridazine-3-carboxylic acid (3-methylbutyl)amide;
6-[4-(2,4-Dichlorobenzoyl)piperazin-1-yl]-pyridazine-3-carboxylic acid (3-methylbutyl)amide;
6-[4-(2-Aminobenzoyl)piperazin-1-yl]-pyridazine-3-carboxylic acid (3-methyl butyl)amide;
6-[4-(2-Trifluoromethylbenzoyl)piperazin-1-yl]-pyridazine-3-carboxylic acid [2-(4-chlorophenoxy)ethyl]amide;
6-[4-(2-Trifluoromethylbenzoyl)piperazin-1-yl]-pyridazine-3-carboxylic acid [2-(4-fluorophenoxy)ethyl]amide;
6-[4-(2-Trifluoromethylbenzoyl)piperazin-1-yl]-pyridazine-3-carboxylic acid (3,3-dimethylbutyl)amide;
6-[4-(2-Trifluoromethylbenzoyl)piperazin-1-yl]-pyridazine-3-carboxylic acid pentylamide;
4-({6-[4-(2-Trifluoromethylbenzoyl)piperazin-1-yl]-pyridazine-3-carbonyl}amino)butyric acid ethyl ester;
6-[4-(5-Fluoro-2-trifluoromethylbenzoyl)piperazin-1-yl]-pyridazine-3-carboxylic acid pentylamide;
6-[4-(2-Trifluoromethylbenzoyl)piperazin-1-yl]-pyridazine-3-carboxylic acid (4-methylpentyl)amide;
6-[4-(2-Fluoro-6-trifluoromethylbenzoyl)piperazin-1-yl]-pyridazine-3-carboxylic acid (3-methylbutyl)amide;
6-[4-(2,6-Difluorobenzoyl)piperazin-1-yl]-pyridazine-3-carboxylic acid (3-methyl butyl)amide;
6-[4-(2-Trifluoromethylbenzoyl)piperazin-1-yl]-pyridazine-3-carboxylic acid (2-oxo-2-phenylethyl)amide;
Acetic acid 1,1-dimethyl-3-({6-[4-(2-trifluoromethyl-benzoyl)piperazin-1-yl]-pyridazine-3-carbonyl}amino)propyl ester;
6-[4-(2-Trifluoromethylbenzoyl)piperazin-1-yl]-pyridazine-3-carboxylic acid (2-phenoxyethyl)amide;
6-[4-(2-Trifluoromethylbenzoyl)piperazin-1-yl]-pyridazine-3-carboxylic acid hexylamide;
6-[4-(2-Trifluoromethylbenzoyl)piperazin-1-yl]-pyridazine-3-carboxylic acid (4-methylpentyl)amide;
6-[4-(5-Fluoro-2-trifluoromethylbenzoyl)piperazin-1-yl]-pyridazine-3-carboxylic acid (4-methylpentyl)amide;
6-[2,5-Dimethyl-4-(2-trifluoromethylbenzoyl)piperazin-1-yl]-pyridazine-3-carboxylic acid pentylamide;
6-[4-(2-Trifluoromethylbenzoyl)-piperazin-1-yl]-pyridazine-3-carboxylic acid heptylamide;
6-[4-(2-Sulfamoylbenzoyl)-piperazin-1-yl]-pyridazine-3-carboxylic acid (3-methylbutyl)-amide;
6-[4-(5-Chloro-2-trifluoromethyl-benzoyl)-piperazin-1-yl]-pyridazine-3-carboxylic acid hexylamide;
6-[4-(2-Trifluoromethylbenzoyl)-piperazin-1-yl]-pyridazine-3-carboxylic acid (2-cyclopropyl-2-oxoethyl)amide;
4-Trifluoromethyl-6-[4-(2-trifluoromethylbenzoyl)-piperazin-1-yl]-pyridazine-3-carboxylic acid (3-methyl-butyl)amide;
6-[4-(5-Fluoro-2-trifluoromethylbenzoyl)piperazin-1-yl]-pyridazine-3-carboxylic acid pentyl-4-enylamide;
6-(4-Benzoylpiperazin-1-yl)-pyridazine-3-carboxylic acid (2-cyclopropylethyl)amide;
6-[4-(2-Chloro-5-fluorobenzoyl)piperazin-1-yl]-pyridazine-3-carboxylic acid (2-cyclopropylethyl)amide;
6-[4-(5-Chloro-2-trifluoromethylbenzoyl)piperazin-1-yl]-pyridazine-3-carboxylic acid (2-cyclopropylethyl)amide;
6-[4-(2,6-Difluorobenzoyl)piperazin-1-yl]-pyridazine-3-carboxylic acid (2-cyclopropylethyl)amide;
6-[4-(2,5-Bis-trifluoromethylbenzoyl)piperazin-1-yl]-pyridazine-3-carboxylic acid (2-cyclopropylethyl)amide;
6-[4-(2,4-Bis-trifluoromethylbenzoyl)piperazin-1-yl]-pyridazine-3-carboxylic acid (2-cyclopropylethyl)amide;
6-[4-(2,5-Difluorobenzoyl)piperazin-1-yl]-pyridazine-3-carboxylic acid (2-cyclopropylethyl)amide;
6-[4-(5-Fluoro-2-trifluoromethylbenzoyl)piperazin-1-yl]-pyridazine-3-carboxylic acid (3-cyclopropylpropyl)amide;
6-[4-(2-Fluorobenzoyl)piperazin-1-yl]-pyridazine-3-carboxylic acid (2-cyclopropylethyl)amide;
6-[4-(3-Fluoro-2-trifluoromethylbenzoyl)piperazin-1-yl]-pyridazine-3-carboxylic acid (2-cyclopropylethyl)amide;
6-[4-(4-Fluoro-2-trifluoromethylbenzoyl)piperazin-1-yl]-pyridazine-3-carboxylic acid (3-cyclopropylpropyl)amide;
6-[4-(2-Trifluoromethylbenzoyl)piperazin-1-yl]-pyridazine-3-carboxylic acid (2-methylcyclopropylmethyl)amide;
6-[4-(5-Fluoro-2-methoxybenzoyl)piperazin-1-yl]-pyridazine-3-carboxylic acid (2-cyclopropylethyl)amide;
6-[4-(2-Dimethylaminobenzoyl)piperazin-1-yl]-pyridazine-3-carboxylic acid (2-cyclopropylethyl)amide;
6-[4-(2-Chloro-5-dimethylaminobenzoyl)piperazin-1-yl]-pyridazine-3-carboxylic acid (2-cyclopropylethyl)amide;
6-[4-(2,5-Dimethylbenzoyl)piperazin-1-yl]-pyridazine-3-carboxylic acid (2-cyclopropylethyl)amide;
6-[4-(2,5-Dichlorobenzoyl)piperazin-1-yl]-pyridazine-3-carboxylic acid (2-cyclopropylethyl)amide;
6-[4-(2-Trifluoromethylbenzoyl)piperazin-1-yl]-pyridazine-3-carboxylic acid cyclobutylmethylamide;
Acetic acid 2-{4-[6-(2-cyclopropylethylcarbamoyl)-pyridazin-3-yl]-piperazine-1-carbonyl}phenyl ester;
6-[4-(2-Trifluoromethylbenzoyl)piperazin-1-yl]-pyridazine-3-carboxylic acid (2-cyclopropyl-2-hydroxyethyl)amide;
6-[4-(2-Trifluoromethylbenzoyl)piperazin-1-yl]-pyridazine-3-carboxylic acid (2-phenylcyclopropylmethyl)amide;
6-[4-(2-Trifluoromethylbenzoyl)piperazin-1-yl]-pyridazine-3-carboxylic acid (3-cyclopropylpropyl)amide;
6-[4-(2-Cyanobenzoyl)piperazin-1-yl]-pyridazine-3-carboxylic acid (2-cyclopropylethyl)amide;
6-{4-[2-(2-Trifluoromethylphenyl)acetyl]-piperazin-1-yl}pyridazine-3-carboxylic acid (2-cyclopropylethyl)amide;
6-[4-(4-Fluoro-2-trifluoromethylbenzoyl)piperazin-1-yl]-pyridazine-3-carboxylic acid (2-cyclopropylethyl)amide;
6-[4-(5-Chloro-2-trifluoromethylbenzoyl)piperazin-1-yl]-pyridazine-3-carboxylic acid (3-cyclopropylpropyl)amide;
6-[3,5-Dimethyl-4-(2-trifluoromethyl benzoyl)piperazin-1-yl]-pyridazine-3-carboxylic acid (2-cyclopropylethyl)amide;
2-{4-[6-(2-Cyclopropylethylcarbamoyl)pyridazin-3-yl]-piperazine-1-carbonyl}benzoic acid methyl ester;
6-[4-(2-Trifluoromethyl-benzoyl)-piperazin-1-yl]-pyridazine-3-carboxylic acid (2-cyclobutylethyl)amide;
2-{4-[6-(2-Cyclopropyl-ethylcarbamoyl)-pyridazin-3-yl]-piperazine-1-carbonyl}-benzoic acid;
6-[4-(5-Chloro-2-trifluoromethyl-benzoyl)-piperazin-1-yl]-pyridazine-3-carboxylic acid (2-cyclobutylethyl)amide;
6-[4-(5-Fluoro-2-trifluoromethylbenzoyl)-piperazin-1-yl]-pyridazine-3-carboxylic acid (2-cyclobutylethyl)amide;
6-[4-(2-Trifluoromethylbenzoyl)-piperazin-1-yl]-pyridazine-3-carboxylic acid (3-cyclobutylpropyl)amide;
6-[4-(5-Fluoro-2-trifluoromethylbenzoyl)-piperazin-1-yl]-pyridazine-3-carboxylic acid (2-cyclopropylethyl)amide;
6-[4-(2-Trifluoromethyl-thiobenzoyl)-piperazin-1-yl]-pyridazine-3-carboxylic acid (2-cyclopropylethyl)amide;
6-[4-(2-Trifluoromethylbenzoyl)-piperazin-1-yl]-pyridazine-3-carboxylic acid (4-cyclopropylbutyl)amide;
6-[4-(2-Trifluoromethylbenzoyl)-piperazin-1-yl]-pyridazine-3-carboxylic acid (2,2-dimethyl-cyclopropylmethyl)amide;
6-[4-(Pyridine-2-carbonyl)piperazin-1-yl]-pyridazine-3-carboxylic acid (2-cyclopropylethyl)amide;
6-[4-(2-Ttrifluoromethylfuran-3-carbonyl)piperazin-1-yl]-pyridazine-3-carboxylic acid (2-cyclopropylethyl)amide;
6-[4-(2-Chloro-4-trifluoromethylpyrimidine-5-carbonyl)piperazin-1-yl]-pyridazine-3-carboxylic acid (2-cyclopropylethyl)amide;
6-[4-(5-Methyl-2-trifluoromethylfuran-3-carbonyl)piperazin-1-yl]-pyridazine-3-carboxylic acid (2-cyclopropylethyl)amide;
6-[4-(2-Chloropyridine-3-carbonyl)piperazin-1-yl]-pyridazine-3-carboxylic acid (2-cyclopropylethyl)amide;
6-[4-(2-Methyl-5-trifluoromethyloxazole-4-carbonyl)piperazin-1-yl]-pyridazine-3-carboxylic acid (2-cyclopropylethyl)amide;
6-[4-(2,6-Dichloropyridine-3-carbonyl)piperazin-1-yl]-pyridazine-3-carboxylic acid (2-cyclopropylethyl)amide;
6-[4-(Pyrrolidine-1-carbonyl)piperazin-1-yl]-pyridazine-3-carboxylic acid (2-cyclopropylethyl)amide;
6-[4-(1-Methyl-1H-pyrrole-2-carbonyl)piperazin-1-yl]-pyridazine-3-carboxylic acid (2-cyclopropylethyl)amide;
6-[4-(Tetrahydrofuran-2-carbonyl)piperazin-1-yl]-pyridazine-3-carboxylic acid (2-cyclopropylethyl)amide;
6-{4-[2-(2-Trifluoromethylphenyl)acetyl]piperazin-1-yl}-pyridazine-3-carboxylic acid (2-cyclopropylethyl)amide;
4-[6-(3-Methylbutylcarbamoyl)pyridazin-3-yl]-piperazine-1-carboxylic acid t-butyl ester;
4-[6-(2-Cyclopropylethylcarbamoyl)pyridazin-3-yl]-piperazine-1-carboxylic acid t-butyl ester;
6-[4-(4,4,4-Trifluoro-3-hydroxy-3-trifluoromethylbutyryl)piperazin-1-yl]-pyridazine-3-carboxylic acid (2-cyclopropylethyl)amide;
6-[4-(4,4,4-Trifluoro-3-hydroxy-3-methylbutyryl)piperazin-1-yl]-pyridazine-3-carboxylic acid (2-cyclopropylethyl)amide;
and 6-[4-(3,3,3-Trifluoro-2-hydroxy-2-methylpropionyl)piperazin-1-yl]-pyridazine-3-carboxylic acid (2-cyclopropylethyl)amide;
6-[4-(1-Hydroxycyclopropanecarbonyl)piperazin-1-yl]-pyridazine-3-carboxylic acid (2-cyclopropylethyl)amide;
6-(4-Cyclobutanecarbonylpiperazin-1-yl)pyridazine-3-carboxylic acid (2-cyclopropylethyl)amide;
6-[4-(2-Trifluoromethylcyclopropanecarbonyl)piperazin-1-yl]-pyridazine-3-carboxylic acid (2-cyclopropylethyl)amide;
6-(4-Cyclohexanecarbonylpiperazin-1-yl)pyridazine-3-carboxylic acid (2-cyclopropylethyl)amide;
6-[4-(2-Methylcyclohexanecarbonyl)piperazin-1-yl]-pyridazine-3-carboxylic acid (2-cyclopropylethyl)amide;
6-[4-(3-Methylcyclohexanecarbonyl)piperazin-1-yl]-pyridazine-3-carboxylic acid (2-cyclopropylethyl)amide;
6-[4-(4-Methylcyclohexanecarbonyl)piperazin-1-yl]-pyridazine-3-carboxylic acid (2-cyclopropylethyl)amide;
6-[4-(2-Methylcyclopropanecarbonyl)piperazin-1-yl]-pyridazine-3-carboxylic acid (2-cyclopropylethyl)amide;
6-[4-(2,2,3,3-Tetramethylcyclopropanecarbonyl)piperazin-1-yl]pyridazine-3-carboxylic acid (2-cyclopropylethyl)amide;
6-[4-(2-Ethylbutyryl)piperazin-1-yl]-pyridazine-3-carboxylic acid (2-cyclopropylethyl)amide;
6-[4-(3,3,3-Trifluoro-2-methyl-2-trifluoromethylpropionyl)piperazin-1-yl]-pyridazine-3-carboxylic acid (2-cyclopropylethyl)amide;
6-[4-(2,2-Dimethylpropionyl)piperazin-1-yl]-pyridazine-3-carboxylic acid (2-cyclopropylethyl)amide;
6-[4-(2,2-Dimethylbutyryl)piperazin-1-yl]-pyridazine-3-carboxylic acid (2-cyclopropylethyl)amide;
6-[4-(2,2-Dimethylpentanoyl)piperazin-1-yl]-pyridazine-3-carboxylic acid (2-cyclopropylethyl)amide;
6-[4-(4,4,4-Trifluorobut-2-enoyl)piperazin-1-yl]-pyridazine-3-carboxylic acid (2-cyclopropylethyl)amide; and
6-[4-(4,4,4-Trifluoro-3-trifluoromethylbut-2-enoyl)-piperazin-1-yl]-pyridazine-3-carboxylic acid (2-cyclopropyl-ethyl)amide;
or a pharmaceutically acceptable salt thereof.

9. A combination according to claim 2, wherein an inhibitor of protein tyrosine phosphatase (PTPase) is a compound of the formula 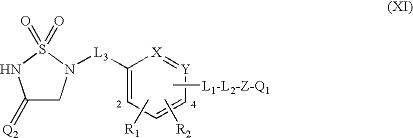
wherein
R₁ is hydrogen, halogen, hydroxy, alkoxy, carboxy, cyano, nitro, trifluoromethyl, alkynyl, alkylthio, heteroaralkyl, heteroaralkoxy or heteroaryloxy provided that R₁ is located at the 2-position when L₃ is —(CHR)ₛ— in which s is zero; or
R₁ is optionally substituted alkyl, alkenyl, optionally substituted amino, aralkyl, aralkoxy, aralkylthio, aryloxy, arylthio or cycloalkyl provided that a monocyclic aryl group which is substituted at the para position with a methylene or ethylene bridged nitrogen containing heterocycle does not constitute part of R₁ when
(i) R₁ is located at the 2-position and L₃ is —(CHR)ₛ— in which s is zero;
(ii) X and Y each are CH; and
(iii) Q₂ is oxygen; or
C—R₁ may be replaced with nitrogen or N→O; or
R₁ and R₂ combined together with the carbon atoms to which R₁ and R₂ are attached form an optionally substituted fused 5- to 6-membered aromatic or heteroaromatic ring provided that R₁ and R₂ are attached to carbon atoms adjacent to each other; or
R₂ is hydrogen, halogen, hydroxy, alkoxy, cyano, trifluoromethyl, nitro, optionally substituted amino, optionally substituted alkyl, alkylthio, aralkyl, heteroaralkyl, aralkoxy, heteroaralkoxy, aralkylthio, aryloxy, heteroaryloxy, arylthio or cycloalkyl; or
R₂ is —C(O)R₃ wherein
R₃ is hydroxy or optionally substituted alkoxy; or
R₃ is —NR₄R₅ in which R₄ and R₅ are independently hydrogen, optionally substituted alkyl, alkenyl, alkynyl, cycloalkyl, aryl, heterocyclyl, aralkyl or heteroaralkyl;
L₁ is a single bond; or
L₁ is carbon which combined together with R₂ and the carbon atoms to which L₁ and R₂ are attached form an optionally substituted fused 5- or 6-membered aromatic or heteroaromatic ring provided that L₁ and R₂ are attached to carbon atoms adjacent to each other; or
L₁ is CH or nitrogen which taken together with R₂ and the carbon atoms to which L₁ and R₂ are attached form a fused 5- to 7-membered ring which may be interrupted with one or two heteroatoms selected from oxygen, nitrogen and sulfur provided that L₁ and R₂ are attached to carbon atoms adjacent to each other; or
L₁ is CH, oxygen, sulfur or nitrogen and L₂ is carbon which combined together with L₁, R₂ and the carbon atoms to which L₁ and R₂ are attached form an optionally substituted fused 5- or 6-membered aromatic or heteroaromatic ring provided that L₁ and R₂ are attached to carbon atoms adjacent to each other; or
L₁ is —CH₂—, oxygen, sulfur or —NR₆— and L₂ is CH which taken together with L₁, R₂ and the carbon atoms to which L₁ and R₂ are attached form a fused 5- to 7-membered ring which may be interrupted with one or two heteroatoms selected from oxygen, nitrogen and sulfur wherein
R₆ is hydrogen, optionally substituted alkyl, aralkyl, heteroaralkyl, alkoxycarbonyl, aryloxycarbonyl, carbamoyl, sulfonyl or acyl provided that L₁ and R₂ are attached to carbon atoms adjacent to each other;
L₂ is —(CHR₇)ₙ— wherein
R₇ is hydrogen, hydroxy, alkoxy, carboxy, optionally substituted alkyl, cycloalkyl, aryl or heteroaryl;
n is zero or an integer from 1 to 4;
Z is —(CHR₈)ₘ—, —(CH₂)ₘO(CHR₈)ᵣ—, —(CH₂)ₘS(CHR₈)ᵣ— or —(CH₂)ₘNR₉(CHR₈)ᵣ— wherein
R₈ is hydrogen, optionally substituted alkyl, cycloalkyl, aryl or heterocyclyl;
R₉ is hydrogen, optionally substituted alkyl, cycloalkyl, aryl, heterocyclyl, aralkyl, heteroaralkyl, alkoxycarbonyl, aryloxycarbonyl, heteroaryloxycarbonyl, carbamoyl, sulfonyl, acyl or acylamino;
m and r are independently zero or an integer of 1 or 2;
Q₁ is hydrogen, optionally substituted alkyl, cycloalkyl, aryl or heterocyclyl provided that
(i) Q₁ is not 2-phenyloxazol-4-yl when
R₁ and R₂ are hydrogen;
X and Y each are CH;
L₁ is a single bond located at the 4-position;
L₂ is —(CHR₇)ₙ— wherein n is zero;
L₃ is —(CHR)ₛ— wherein s is zero;
Z is —(CH₂)ₘO(CHR₈)ᵣ— wherein R₈ is hydrogen, m is zero and r is 2; and
Q₂ is oxygen; or
(ii) Q₁ is not hydrogen when
R₁ and R₂ are hydrogen;
X and Y each are CH;
L₁ is a single bond;
L₂ is —(CHR₇)ₙ— wherein n is zero;
L₃ is —(CHR)ₛ— wherein R is hydrogen and s is 1;
Z is —(CHR₈)ₘ— wherein m is zero; and
Q₂ is oxygen; or
Q₁ is —C(O)NR₄ₐR₅ₐ, —C(O)R₁₀, —C(O)OR₁₀ or —S(O)_{q}R₁₀ wherein R₄ₐ and R₅ₐ are as defined for R₄ and R₅; R₁₀ is optionally substituted alkyl, cycloalkyl, aryl, heterocyclyl, aralkyl or heteroaralkyl; q is an integer of 1 or 2; or
Q₁ is a radical of the formula 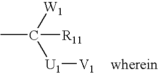
W₁ is aryl, heteroaryl, aralkyl or heteroaralkyl; or
W₁ is —C(O)R₃ₐ in which R₃ₐ is hydroxy or optionally substituted alkoxy; or
R₃ₐ is —NR₄ₐR₅ₐ in which R₄ₐ and R₅ₐ are as defined for R₄ and R₅;
R₁₁ is hydrogen, alkyl or aryl;
U₁ is —C(O)—, —S(O)₂— or —(CH₂)ᵣ— in which r is as defined for Z;
V₁ is hydroxy, alkoxy, aryl, heteroaryl, optionally substituted alkyl or cycloalkyl; or
V₁ is —NR_{4b}R_{5b} in which R_{4b} and R_{5b} are as defined for R₄ and R₅ provided that
(i) L₂ is —(CHR₇)ₙ— in which n is an integer of 1 or 2; and
(ii) Z is —(CHR₈)ₘ— in which m is zero; or
Q₁ is a radical of the formula 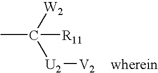
W₂ is —C(O)R₃ₐ in which R₃ₐ is hydroxy or optionally substituted alkoxy; or
R₃ₐ is —NR₄ₐR₅ₐ in which R₄ₐ and R₅ₐ are as defined for R₄ and R₅;
R₁₁ is hydrogen, alkyl or aryl;
U₂ is —(CH₂)ₚ— in which p is zero or 1;
V₂ is —NR_{4b}C(O)R_{5b}, —NR_{4b}C(O)OR_{5b}, —NR_{4b}C(O)NR_{4c}R_{5b} or —NR_{4b}S(O)₂R_{5b} in which
R_{4b} and R_{4c} are as defined for R₄, and R_{5b} has a meaning as defined for R₅ provided that
(i) L₂ is —(CHR₇)ₙ— in which n is an integer of 1 or 2; and
(ii) Z is —(CHR₈)ₘ— in which m is zero; or
Q₁ is a radical of the formula 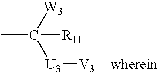
W₃ is —C(O)R₃ₐ in which R₃ₐ is hydroxy or optionally substituted alkoxy; or
R₃ₐ is —NR₄ₐR₅ₐ in which R₄ₐ and R₅ₐ are as defined for R₄ and R₅;
R₁₁ is hydrogen, alkyl or aryl;
U₃ is —(CH₂)ₚ— in which p is zero or 1;
V₃ is —NHC(O)CHR_{4b}NHC(O)R₁₂ wherein R_{4b} is as defined for R₄; R₁₂ is hydrogen, aryl, heterocyclyl, aralkyl, heteroaralkyl, optionally substituted alkyl, alkoxy or cycloalkyl; or
R₁₂ is —NR_{4c}R_{5b} in which R₄, and R_{5b} are as defined for R₄ and R₅ provided that
(i) L₂ is —(CHR₇)ₙ— in which n is an integer of 1 or 2; and
(ii) Z is —(CHR₈)ₘ— in which m is zero;
L₃ is —(CHR)ₙ— wherein
R is hydrogen, carboxy, optionally substituted alkyl, cycloalkyl, aryl or heteroaryl;
s is zero or an integer from 1 to 3;
Q₂ is oxygen, sulfur or NR₁₃ wherein
R₁₃ is hydrogen, hydroxy or lower alkyl;
X and Y are independently CH or nitrogen; or
—X═Y— is sulfur, oxygen or —NR₁₄— wherein
R₁₄ is hydrogen, optionally substituted alkyl, alkoxycarbonyl, acyl, aryloxycarbonyl, heteroaryloxycarbonyl, carbamoyl or sulfonyl;
or a pharmaceutically acceptable salt thereof; or a prodrug derivative thereof.

10. A pharmaceutical composition comprising a renin inhibitor, or a pharmaceutically acceptable salt thereof, and at least one therapeutic agent selected from the group consisting of
(a) an insulin secretion enhancer, or a pharmaceutically acceptable salt thereof; and
(b) an insulin sensitizer, or a pharmaceutically acceptable salt thereof;
and a pharmaceutically acceptable carrier.

11. A pharmaceutical composition according to claim 10, wherein an insulin secretion enhancer is a glucokinase (GK) activator and an insulin sensitizer is selected from the group consisting of an inhibitor of stearoyl-CoA desaturase-1 (SCD-1), an inhibitor of diacylglycerol acyltransferase 1 (DGAT1), an inhibitor of diacylglycerol acyltransferase 2 (DGAT2) and an inhibitor of phosphotyrosine phosphatase (PTPase).

12. A pharmaceutical composition according to claim 11, wherein a renin inhibitor is selected from the group consisting of RO 66-1132, RO 66-1168 and a compound of the formula 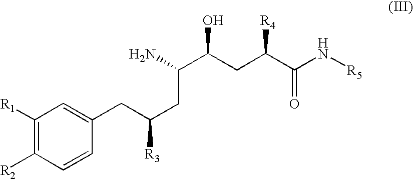
wherein R₁ is halogen, C₁₋₆halogenalkyl, C₁₋₆alkoxy-C₁₋₆alkyloxy or C₁₋₆alkoxy-C₁₋₆alkyl; R₂ is halogen, C₁₋₄alkyl or C₁₋₄alkoxy; R₃ and R₄ are independently branched C₃₋₆alkyl; and R₅ is cycloalkyl, C₁₋₆alkyl, C₁₋₆hydroxyalkyl, C₁₋₆alkoxy-C₁₋₆alkyl, C₁₋₆alkanoyloxy-C₁₋₆alkyl, C₁₋₆aminoalkyl, C₁₋₆alkylamino-C₁₋₆alkyl, C₁₋₆dialkylamino-C₁₋₆alkyl, C₁₋₆alkanoylamino-C₁₋₆alkyl, HO(O)C—C₁₋₆alkyl, C₁₋₆alkyl-O—(O)C—C₁₋₆alkyl, H₂N—C(O)—C₁₋₆alkyl, C₁₋₆alkyl-HN—C(O)—C₁₋₆alkyl or (C₁₋₆alkyl)₂N—C(O)—C₁₋₆alkyl; or a pharmaceutically acceptable salt thereof.

13. A pharmaceutical composition according to claim 12, wherein a renin inhibitor is a compound of formula (III) having the formula 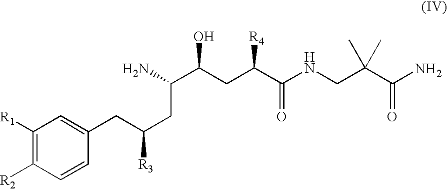
wherein R₁ is 3-methoxypropyloxy; R₂ is methoxy; and R₃ and R₄ are isopropyl; or a pharmaceutically acceptable salt thereof.

14. A pharmaceutical composition according to claim 13, wherein the compound of formula (IV) is in the form of the hemi-fumarate salt thereof.

15. A pharmaceutical composition according to claim 11, wherein a glucokinase activator is selected from the group consisting of 6-[(3-isobutoxy-5-isopropoxybenzoyl)amino]nicotinic acid (GKA1), 5-({3-isopropoxy-5-[2-(3-thienyl)ethoxy]benzoyl}amino)-1,3,4-thiadiazole-2-carboxylic acid (GKA2), 2-(S)-Cyclohexyl-1-(R)-(4-methanesulfonyl-phenyl)-cyclopropanecarboxylic acid thiazol-2-ylamide (LY2121260) and RO-28-1675 or a pharmaceutically acceptable salt thereof.

16. A pharmaceutical composition according to claim 11, wherein a glucokinase activator is a compound of the formula
R—NH-Q (IX)
wherein
(i) Q is a 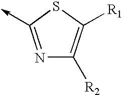
radical in which R₁ and R₂ are independently hydrogen or halogen; or
Q is a 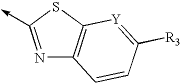
radical in which R₃ is hydrogen, halogen, alkyl, cycloalkyl, aryl, alkoxy, cycloalkoxy, aryloxy, alkylthio, cycloalkylthio, arylthio, acyl, sulfonyl, alkylamino, cycloalkylamino, arylamino, acylamino, sulfonamido or alkoxycarbonyl; Y is CH or nitrogen; and
R is a radical of the formula 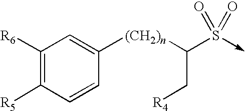
wherein
R₄ is C₂₋₄alkyl, C₃₋₇cycloalkyl or C₅₋₇heterocycloalkyl;
R₅ and R₆ are independently hydrogen, halogen, cyano, R₇, —C(O)R₇ or —S(O)₂R₇ wherein
R₇ is —(CR₈R₉)ₘ—W—R₁₀ in which
R₈ and R₉ are independently hydrogen or lower alkyl;
W is a bond, O, S or —NR₁₁ in which
R₁₁ is hydrogen or lower alkyl;
R₁₀ is hydrogen, alkyl, cycloalkyl, aryl or heterocyclyl; or R₁₀ and R₁₁, combined, are alkylene which together with the nitrogen atom to which they are attached form a 5- to 7-membered ring;
m is zero or an integer from 1 to 5;
n is zero or an integer of 1 or 2;
or an optical isomer thereof; or a pharmaceutically acceptable salt thereof; or
(ii) Q is a 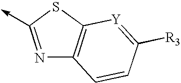
radical in which R₃ is hydrogen, halogen, alkyl, cycloalkyl, aryl, alkoxy, cycloalkoxy, aryloxy, alkylthio, cycloalkylthio, arylthio, acyl, sulfonyl, alkylamino, cycloalkylamino, arylamino, acylamino, sulfonamido or alkoxycarbonyl; and
R is a radical of the formula 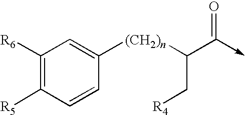
wherein
R₄ is C₂₋₄alkyl, C₃₋₇cycloalkyl or C₅₋₇heterocycloalkyl;
R₅ and R₆ are independently hydrogen, halogen, cyano, R₇, —C(O)R₇ or —S(O)₂R₇ wherein
R₇ is —(CR₈R₉)ₘ—W—R₁₀ in which
R₈ and R₉ are independently hydrogen or lower alkyl;
W is a bond, O, S or —NR₁₁ in which
R₁₁ is hydrogen or lower alkyl;
R₁₀ is hydrogen, alkyl, cycloalkyl, aryl or heterocyclyl; or R₁₀ and R₁₁, combined, are alkylene which together with the nitrogen atom to which they are attached form a 5- to 7-membered ring;
m is zero or an integer from 1 to 5;
n is zero or an integer of 1 or 2;
or an optical isomer thereof; or a pharmaceutically acceptable salt thereof; or
(iii) Q is a 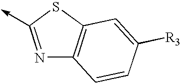
radical in which R₃ is hydrogen, halogen, alkyl, cycloalkyl, aryl, alkoxy, cycloalkoxy, aryloxy, alkylthio, cycloalkylthio, arylthio, acyl, sulfonyl, alkylamino, cycloalkylamino, arylamino, acylamino, sulfonamido or alkoxycarbonyl; and
R is a radical of the formula 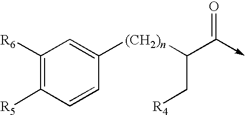
wherein
R₄ is C₂₋₄alkyl, C₃₋₇cycloalkyl or C₅₋₇heterocycloalkyl;
R₅ and R₆ are independently hydrogen, halogen, cyano, R₇, —C(O)R₇ or —S(O)₂R₇ wherein
R₇ is —(CR₈R₉)ₘ—W—R₁₀ in which
R₈ and R₉ are independently hydrogen or lower alkyl;
W is a bond, O, S or —NR₁₁ in which
R₁₁ is hydrogen or lower alkyl;
R₁₀ is hydrogen, alkyl, cycloalkyl, aryl or heterocyclyl; or R₁₀ and R₁₁, combined, are alkylene which together with the nitrogen atom to which they are attached form a 5- to 7-membered ring;
m is zero or an integer from 1 to 5;
n is zero or an integer of 1 or 2;
or an optical isomer thereof; or a pharmaceutically acceptable salt thereof; or
(iv) Q is a 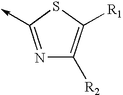
radical, wherein R₁ and R₂ are independently hydrogen or halogen; and
R is a radical of the formula 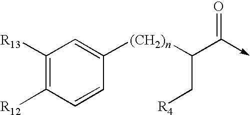
wherein
R₄ is C₂₋₄alkyl, C₃₋₇cycloalkyl or C₅₋₇heterocycloalkyl;
R₁₂ and R₁₃ are independently hydrogen, halogen, cyano, R₁₄, —C(O)R₁₄, or —S(O)₂R₁₄ wherein
R₁₄ is —(CR₈R₉)ₘ—W—R₁₅ in which
R₈ and R₉ are independently hydrogen or lower alkyl;
W is a bond, O, S or —NR₁₁ in which
R₁₁ is hydrogen or lower alkyl;
R₁₅ is cycloalkyl, aryl or heterocyclyl; or R₁₅ and R₁₁, combined, are alkylene which together with the nitrogen atom to which they are attached form a 5- to 7-membered ring;
m is zero or an integer from 1 to 5;
n is zero or an integer of 1 or 2;
or an optical isomer thereof; or a pharmaceutically acceptable salt thereof.

17. A pharmaceutical composition according to claim 11, wherein an inhibitor of stearoyl-CoA desaturase (SCD-1) is selected from the group consisting of
1-Pentyl-3-{6-[4-(2-trifluoromethylbenzoyl)piperazin-1-yl]pyridazin-3-yl}urea;
1-Benzyl-3-{6-[4-(2-trifluoromethylbenzoyl)piperazin-1-yl]pyridazin-3-yl}urea;
1-(4-Fluorophenyl)-3-{6-[4-(2-trifluoromethylbenzoyl)piperazin-1-yl]-pyridazin-3-yl}urea;
1-(2-Fluorophenyl)-3-{6-[4-(2-trifluoromethylbenzoyl)piperazin-1-yl]pyridazin-3-yl}urea;
1-[1-(4-Fluorophenyl)ethyl]-3-{6-[4-(2-trifluoromethylbenzoyl)-piperazin-1-yl]pyridazin-3-yl}urea;
1-[1-(4-Fluorophenyl)ethyl]-3-{6-[4-(2-trifluoromethylbenzoyl)piperazin-1-yl]pyridazin-3-yl}urea;
1-[3-(4-Fluorophenyl)propyl]-3-{6-[4-(2-trifluoromethylbenzoyl)piperazin-1-yl]pyridazin-3-yl}urea;
1-Phenethyl-3-{6-[4-(2-trifluoromethylbenzoyl)piperazin-1-yl]-pyridazin-3-yl}urea;
1-(4-Fluorobenzyl)-3-{6-[4-(2-trifluoromethylbenzoyl)piperazin-1-yl]-pyridazin-3-yl}urea;
1-(3,4-Dichlorobenzyl)-3-{6-[4-(2-trifluoromethylbenzoyl)piperazin-1-yl]-pyridazin-3-yl}urea;
1-(2-Phenylcyclopropyl)-3-{6-[4-(2-trifluoromethylbenzoyl)piperazin-1-yl]-pyridazin-3-yl}urea;
1-Cyclopentyl-3-{6-[4-(2-trifluoromethylbenzoyl)piperazin-1-yl]-pyridazin-3-yl}urea;
1-(3-Cyclopropylpropyl)-3-{6-[4-(2-trifluoromethylbenzoyl)piperazin-1-yl]-pyridazin-3-yl}urea;
1-Cyclopropylmethyl-3-{6-[4-(2-trifluoromethylbenzoyl)piperazin-1-yl]-pyridazin-3-yl}urea;
1-(2-Cyclopropylethyl)-3-{6-[4-(2-trifluoromethylbenzoyl)-piperazin-1-yl]-pyridazin-3-yl}urea;
1-(2-Cyclopropylethyl)-3-{6-[4-(2-fluoro-6-trifluoromethylbenzoyl)piperazin-1-yl]-pyridazin-3-yl}urea;
1-(2-Cyclopropylethyl)-3-{6-[4-(5-fluoro-2-trifluoromethylbenzoyl)piperazin-1-yl]-pyridazin-3-yl}urea;
1-Cyclohexyl-3-{6-[4-(2-trifluoromethylbenzoy-l)piperazin-1-yl]-pyridazin-3-yl}urea;
1-(2-Cyclopropylethyl)-3-{6-[4-(2,6-difluorobenzoyl)piperazin-1-yl]-pyridazin-3-yl}urea;
1-(3-Cyclopropylpropyl)-3-{6-[4-(5-fluoro-2-trifluoromethylbenzoyl)piperazin-1-yl]-pyridazin-3-yl}urea;
4-Phenyl-N-{6-[4-(2-trifluoromethylbenzoyl)piperazin-1-yl]-pyridazin-3-yl}butyramide;
4-Methylpentanoic acid {6-[4-(2-trifluoromethylbenzoyl)-piperazin-1-yl]-pyridazin-3-yl}amide;
3-Cyclopentyl-N-{6-[4-(2-trifluoromethylbenzoyl)piperazin-1-yl]-pyridazin-3-yl}propionamide;
6-[4-(2-Trifluoromethylbenzoyl)piperazin-1-yl]-pyridazine-3-carboxylic acid phenethylamide;
6-[4-(2-Trifluoromethylbenzoyl)piperazin-1-yl]-pyridazine-3-carboxylic acid [2-(4-methoxyphenyl)ethyl]amide;
6-[4-(2-Trifluoromethylbenzoyl)piperazin-1-yl]-pyridazine-3-carboxylic acid [2-(3-fluorophenyl)ethyl]amide;
6-[4-(2-Trifluoromethylbenzoyl)piperazin-1-yl]-pyridazine-3-carboxylic acid (3-phenylpropyl)amide;
6-[4-(2-Trifluoromethyl benzoyl)piperazin-1-yl]-pyridazine-3-carboxylic acid [2-(4-fluorophenyl)ethyl]amide;
4-Methyl-2-({6-[4-(2-trifluoromethylbenzoyl)piperazin-1-yl]-pyridazine-3-carbonyl}amino)pentanoic acid methyl ester;
6-[4-(2-Trifluoromethylbenzoyl)piperazin-1-yl]-pyridazine-3-carboxylic acid (3-methylbutyl)amide;
6-[4-(5-Fluoro-2-trifluoromethylbenzoyl)piperazin-1-yl]-pyridazine-3-carboxylic acid (3-methylbutyl)amide;
6-[4-(4-Fluoro-2-trifluoromethylbenzoyl)piperazin-1-yl]-pyridazine-3-carboxylic acid (3-methylbutyl)amide;
4-Methyl-2-({6-[4-(2-trifluoromethylbenzoyl)piperazin-1-yl]-pyridazine-3-carbonyl}amino)pentanoic acid;
6-[4-(2-Trifluoromethylbenzoyl)piperazin-1-yl]-pyridazine-3-carboxylic acid (2-cyclopropylethyl)amide;
6-[4-(5-Fluoro-2-trifluoromethylbenzoyl)piperazin-1-yl]-pyridazine-3-carboxylic acid (2-cyclopropylethyl)amide;
6-[4-(2-Trifluoromethylbenzoyl)piperazin-1-yl]-pyridazine-3-carboxylic acid cyclopropylmethylamide;
4-(4-Methoxyphenyl)-N-{6-[4-(2-trifluoromethylbenzoyl)-piperazin-1-yl]-pyridazin-3-yl}butyramide;
3-(4-Fluorophenyl)-N-{6-[4-(2-trifluoromethylbenzoyl)-piperazin-1-yl]-pyridazin-3-yl}propionamide;
4-Cyclohexyl-N-{6-[4-(2-trifluoromethylbenzoyl)piperazin-1-yl]-pyridazin-3-yl}butyramide;
2,2,3,3-Tetramethylcyclopropanecarboxylic acid {6-[4-(2-trifluoromethylbenzoyl)piperazin-1-yl]pyridazin-3-yl}amide;
Cyclopropanecarboxylic acid {6-[4-(2-trifluoromethylbenzoyl)piperazin-1-yl]-pyridazin-3-yl}amide;
1-Trifluoromethylcyclopropanecarboxylic acid {6-[4-(2-trifluoromethylbenzoyl)piperazin-1-yl]-pyridazin-3-yl}amide;
2-Phenylcyclopropanecarboxylic acid {6-[4-(2-trifluoromethylbenzoyl)piperazin-1-yl]-pyridazin-3-yl}amide;
6-[4-(2-Trifluoromethylbenzoyl)piperazin-1-yl]-pyridazine-3-carboxylic acid indan-1-ylamide;
6-[4-(2-Trifluoromethyl benzoyl)piperazin-1-yl]-pyridazine-3-carboxylic acid (2-oxo-1,3-diaza-bicyclo[3.1.0]hex-3-en-4-yl)amide;
6-[4-(2-Trifluoromethylbenzoyl)piperazin-1-yl]-pyridazine-3-carboxylic acid (5-oxo-4,5-dihydro-1H-pyrazol-3-yl)amide;
6-[4-(2-Trifluoromethylbenzoyl)piperazin-1-yl]-pyridazine-3-carboxylic acid indan-5-ylamide;
5-[1,2]Dithiolan-3-yl-pentanoic acid {6-[4-(2-trifluoromethyl-benzoyl)-piperazin-1-yl]-pyridazin-3-yl}amide;
6-[4-(2-Trifluoromethyl-benzoyl)-piperazin-1-yl]-pyridazine-3-carboxylic acid (2-thiophen-2-yl-ethyl)amide;
6-[4-(2-Trifluoromethylbenzoyl)piperazin-1-yl]-pyridazine-3-carboxylic acid (2-benzo[1,3]dioxol-5-yl-ethyl)amide;
6-[4-(2-Trifluoromethylbenzoyl)piperazin-1-yl]-pyridazine-3-carboxylic acid (2,2-difluoro-2-pyridin-2-ylethyl)amide;
6-[4-(2-Trifluoromethylbenzoyl)piperazin-1-yl]-pyridazine-3-carboxylic acid (2-pyridin-2-ylethyl)amide;
6-[4-(2-Trifluoromethylbenzoyl)piperazin-1-yl]-pyridazine-3-carboxylic acid (pyridin-2-yl-methyl)amide;
6-[4-(2-Trifluoromethylbenzoyl)piperazin-1-yl]-pyridazine-3-carboxylic acid (5-chloropyridin-2-yl)amide;
6-[4-(2-Trifluoromethylbenzoyl)piperazin-1-yl]-pyridazine-3-carboxylic acid (5-trifluoromethylpyridin-2-yl)-amide;
6-[4-(2-Trifluoromethylbenzoyl)piperazin-1-yl]-pyridazine-3-carboxylic acid (7H-purin-6-yl)amide;
6-[4-(2-Trifluoromethylbenzoyl)piperazin-1-yl]-pyridazine-3-carboxylic acid pyrazin-2-ylamide;
6-[4-(2-Trifluoromethyl benzoyl)piperazin-1-yl]-pyridazine-3-carboxylic acid (1H-tetrazol-5-yl)amide;
6-[4-(2-Trifluoromethylbenzoyl)piperazin-1-yl]-pyridazine-3-carboxylic acid (2H-[1,2,4]triazol-3-yl)amide;
6-[4-(2-Trifluoromethylbenzoyl)piperazin-1-yl]-pyridazine-3-carboxylic acid (3-methylisoxazol-5-yl)amide;
6-[4-(2-Trifluoromethylbenzoyl)piperazin-1-yl]-pyridazine-3-carboxylic acid (5-methylisoxazol-3-yl)amide;
6-[4-(2-Trifluoromethylbenzoyl)piperazin-1-yl]-pyridazine-3-carboxylic acid (1H-pyrazol-3-yl)amide;
6-[4-(2-Trifluoromethylbenzoyl)piperazin-1-yl]-pyridazine-3-carboxylic acid (5-methyl-1H-pyrazol-3-yl)amide;
6-[4-(2-Trifluoromethyl benzoyl)piperazin-1-yl]-pyridazine-3-carboxylic acid pyrimidin-2-ylamide;
6-[4-(2-Trifluoromethylbenzoyl)piperazin-1-yl]-pyridazine-3-carboxylic acid pyrazin-2-ylamide;
6-[4-(2-Trifluoromethylben-zoyl)piperazin-1-yl]-pyridazine-3-carboxylic acid (4-methylpyrimidin-2-yl)amide;
6-[4-(2-Trifluoromethylbenzoyl)piperazin-1-yl]-pyridazine-3-carboxylic acid (2-oxo-2,3-dihydro-pyrimidin-4-yl)amide;
6-[4-(2-Trifluoromethylbenzoyl)piperazin-1-yl]-pyridazine-3-carboxylic acid (6-oxo-1,6-dihydro-pyrimidin-2-yl)amide;
6-[4-(2-Trifluoromethylbenzoyl)piperazin-1-yl]-pyridazine-3-carboxylic acid [1,3,4]thiadiazol-2-ylamide;
6-[4-(2-Trifluoromethylbenzoyl)piperazin-1-yl]-pyridazine-3-carboxylic acid thiazol-2-ylamide;
6-[4-(2-Trifluoromethylbenzoyl)piperazin-1-yl]-pyridazine-3-carboxylic acid pyridin-2-ylamide;
6-[4-(2-Trifluoromethylbenzoyl)piperazin-1-yl]-pyridazine-3-carboxylic acid pyridazin-3-ylamide;
6-[4-(2-Trifluoromethylbenzoyl)piperazin-1-yl]-pyridazine-3-carboxylic acid pyridin-3-ylamide;
6-[4-(2-Trifluoromethylbenzoyl)piperazin-1-yl]-pyridazine-3-carboxylic acid pyridin-4-ylamide;
6-[4-(2-Trifluoromethylbenzoy-l)piperazin-1-yl]-pyridazine-3-carboxylic acid (6-oxo-1,6-dihydro-[1,3,5]triazin-2-yl)amide;
6-[4-(2-Trifluoromethylbenzoyl)piperazin-1-yl]-pyridazine-3-carboxylic acid (5-fluoropyridin-2-yl)amide;
6-[4-(2-Trifluoromethylbenzoyl)-piperazin-1-yl]-pyridazine-3-carboxylic acid (5-cyanopyridin-2-yl)amide;
6-[4-(2-Trifluoromethylbenzoyl)-piperazin-1-yl]-pyridazine-3-carboxylic acid (4,6-dimethyl-pyrimidin-2-yl)amide;
6-[4-(2-Trifluoromethylbenzoyl)-piperazin-1-yl]-pyridazine-3-carboxylic acid (2-chloropyridin-4-yl)amide;
6-[4-(2-Trifluoromethylbenzoyl)-piperazin-1-yl]-pyridazine-3-carboxylic acid (1H-indol-6-yl)amide;
6-[4-(2-Trifluoromethylbenzoyl)-piperazin-1-yl]-pyridazine-3-carboxylic acid (1H-indol-4-yl)amide;
6-[4-(2-Trifluoromethylbenzoyl)-piperazin-1-yl]-pyridazine-3-carboxylic acid (1H-indazol-5-yl)amide;
6-[4-(2-Trifluoromethylbenzoyl)-piperazin-1-yl]-pyridazine-3-carboxylic acid (1H-indazol-6-yl)amide;
6-[4-(2-Trifluoromethyl benzoyl)-piperazin-1-yl]-pyridazine-3-carboxylic acid (4-methylthiazol-2-yl)amide;
6-[4-(2-Trifluoromethylbenzoyl)-piperazin-1-yl]-pyridazine-3-carboxylic acid (5-methylthiazol-2-yl)amide;
6-[4-(2-Trifluoromethylbenzoyl)-piperazin-1-yl]-pyridazine-3-carboxylic acid (5-thioxo-4,5-dihydro-1H-[1,2,4]triazol-3-yl)amide;
6-[4-(2-Trifluoromethylbenzoyl)piperazin-1-yl]-pyridazine-3-carboxylic acid (1H-benzoimidazol-2-yl)amide;
6-[4-(2-Trifluoromethyl benzoyl)piperazin-1-yl]-pyridazine-3-carboxylic acid (6-methylpyridazin-3-yl)amide;
6-[4-(2-Trifluoromethylbenzoyl)piperazin-1-yl]-pyridazine-3-carboxylic acid (6-methoxypyridazin-3-yl)amide;
6-[4-(2-Trifluoromethylbenzoyl)-piperazin-1-yl]-pyridazine-3-carboxylic acid (6-chloropyridazin-3-yl)amide;
6-[4-(2-Trifluoromethyl benzoyl)piperazin-1-yl]-pyridazine-3-carboxylic acid (4-chlorophenyl)amide;
6-[4-(2-Trifluoromethylbenzoyl)piperazin-1-yl]-pyridazine-3-carboxylic acid (4-carbamoyl-phenyl)amide;
6-[4-(2-Trifluoromethylbenzoyl)piperazin-1-yl]-pyridazine-3-carboxylic acid (3-carbamoyl-phenyl)amide;
6-[4-(2-Trifluoromethylbenzoyl)piperazin-1-yl]-pyridazine-3-carboxylic acid m-tolylamide;
6-[4-(2-Trifluoromethylbenzoyl)piperazin-1-yl]-pyridazine-3-carboxylic acid p-tolylamide;
6-[4-(2-Trifluoromethylbenzoyl)piperazin-1-yl]-pyridazine-3-carboxylic acid o-tolylamide;
6-[4-(2-Trifluoromethylbenzoyl)piperazin-1-yl]-pyridazine-3-carboxylic acid (2-propylphenyl)amide;
6-[4-(2-Trifluoromethylbenzoyl)piperazin-1-yl]-pyridazine-3-carboxylic acid (4-propylphenyl)amide;
6-[4-(2-Trifluoromethylbenzoyl)piperazin-1-yl]-pyridazine-3-carboxylic acid (4-isopropylphenyl)amide;
6-[4-(2-Trifluoromethylbenzoyl)piperazin-1-yl]-pyridazine-3-carboxylic acid (2-isopropylphenyl)amide;
6-[4-(2-Trifluoromethylbenzoyl)piperazin-1-yl]-pyridazine-3-carboxylic acid (2-chlorophenyl)amide;
6-[4-(2-Trifluoromethylbenzoyl)piperazin-1-yl]-pyridazine-3-carboxylic acid (2-cyano-3-fluorophenyl)amide;
6-[4-(2-Trifluoromethylbenzoyl)piperazin-1-yl]-pyridazine-3-carboxylic acid (2,4-dimethylphenyl)amide;
6-[4-(2-Trifluoromethylbenzoyl)piperazin-1-yl]-pyridazine-3-carboxylic acid (2,5-dimethylphenyl)amide;
6-[4-(2-Trifluoromethylbenzoyl)piperazin-1-yl]-pyridazine-3-carboxylic acid (2,6-dimethylphenyl)amide;
6-[4-(2-Trifluoromethylbenzoyl)piperazin-1-yl]-pyridazine-3-carboxylic acid (2,3-dimethylphenyl)amide;
6-[4-(2-Trifluoromethylbenzoyl)piperazin-1-yl]-pyridazine-3-carboxylic acid (3,5-dimethylphenyl)amide;
6-[4-(2-Trifluoromethylbenzoyl)piperazin-1-yl]-pyridazine-3-carboxylic acid (3,4-dimethylphenyl)amide;
6-[4-(2-Trifluoromethylbenzoyl)piperazin-1-yl]-pyridazine-3-carboxylic acid (4-ethyl phenyl)amide;
6-[4-(2-Trifluoromethylbenzoyl)piperazin-1-yl]-pyridazine-3-carboxylic acid (2-ethylphenyl)amide;
6-[4-(2-Trifluoromethylbenzoyl)piperazin-1-yl]-pyridazine-3-carboxylic acid (3-fluoro-2-methylphenyl)amide;
6-[4-(2-Trifluoromethyl benzoyl)piperazin-1-yl]-pyridazine-3-carboxylic acid (2-fluoro-4-methylphenyl)amide;
6-[4-(2-Trifluoromethylbenzoyl)piperazin-1-yl]-pyridazine-3-carboxylic acid (4-fluoro-2-methylphenyl)amide;
6-[4-(2-Trifluoromethylbenzoyl)piperazin-1-yl]-pyridazine-3-carboxylic acid (2-fluoro-5-methylphenyl)amide;
6-[4-(2-Trifluoromethylbenzoyl)piperazin-1-yl]-pyridazine-3-carboxylic acid (3-fluoro-5-methylphenyl)amide;
6-[4-(2-Trifluoromethylbenzoyl)piperazin-1-yl]-pyridazine-3-carboxylic acid (3-fluorophenyl)amide;
6-[4-(2-Trifluoromethylbenzoyl)piperazin-1-yl]-pyridazine-3-carboxylic acid (2-fluorophenyl)amide;
6-[4-(2-Trifluoromethylbenzoyl)piperazin-1-yl]-pyridazine-3-carboxylic acid (4-fluorophenyl)amide;
6-[4-(2-Trifluoromethylbenzoyl)piperazin-1-yl-pyridazine-3-carboxylic acid (2,4-difluorophenyl)amide;
6-[4-(2-Trifluoromethylbenzoyl)piperazin-1-yl]-pyridazine-3-carboxylic acid (2,5-difluorophenyl)amide;
6-[4-(2-Trifluoromethylbenzoyl)piperazin-1-yl]-pyridazine-3-carboxylic acid (3,4-difluorophenyl)amide;
6-[4-(2-Trifluoromethylbenzoyl)piperazin-1-yl]-pyridazine-3-carboxylic acid (2,3-difluorophenyl)amide;
6-[4-(2-Trifluoromethylbenzoyl)piperazin-1-yl]-pyridazine-3-carboxylic acid (2,6-difluorophenyl)amide;
6-[4-(2-Trifluoromethylbenzoyl)piperazin-1-yl]-pyridazine-3-carboxylic acid (4-cyanophenyl)amide;
6-[4-(2-Trifluoromethylbenzoyl)piperazin-1-yl]-pyridazine-3-carboxylic acid (2-cyanophenyl)amide;
6-[4-(2-Trifluoromethyl benzoyl)piperazin-1-yl]-pyridazine-3-carboxylic acid (3-cyanophenyl)amide;
6-[4-(2-Trifluoromethylbenzoyl)piperazin-1-yl]-pyridazine-3-carboxylic acid (3-chlorophenyl)amide;
6-[4-(2-Trifluoromethylbenzoyl)piperazin-1-yl]-pyridazine-3-carboxylic acid (3-chloro-2-methylphenyl)amide;
6-[4-(2-Trifluoromethylbenzoyl)piperazin-1-yl]-pyridazine-3-carboxylic acid (2-chloro-3-methylphenyl)amide;
6-[4-(2-Trifluoromethylbenzoyl)piperazin-1-yl]-pyridazine-3-carboxylic acid (2,5-dichlorophenyl)amide;
6-[4-(2-Trifluoromethylbenzoyl)piperazin-1-yl]-pyridazine-3-carboxylic acid (2-chloro-5-methylphenyl)amide;
6-[4-(2-Trifluoromethyl benzoyl)piperazin-1-yl]-pyridazine-3-carboxylic acid (2-chloro-6-methylphenyl)amide;
6-[4-(2-Trifluoromethylbenzoyl)piperazin-1-yl]-pyridazine-3-carboxylic acid (4-chloro-2-methylphenyl)amide;
6-[4-(2-Trifluoromethylbenzoyl)piperazin-1-yl]-pyridazine-3-carboxylic acid (4-chloro-3-methylphenyl)amide;
6-[4-(2-Trifluoromethylbenzoyl)piperazin-1-yl]-pyridazine-3-carboxylic acid (3-chloro-4-methylphenyl)amide;
6-[4-(2-Trifluoromethylbenzoyl)piperazin-1-yl]-pyridazine-3-carboxylic acid (2-chloro-4-methylphenyl)amide;
6-[4-(2-Trifluoromethylbenzoyl)piperazin-1-yl]-pyridazine-3-carboxylic acid (2-chloro-5-fluorophenyl)amide;
6-[4-(2-Trifluoromethylbenzoyl)piperazin-1-yl]-pyridazine-3-carboxylic acid (5-chloro-2-fluorophenyl)amide;
6-[4-(2-Trifluoromethylbenzoyl)piperazin-1-yl]-pyridazine-3-carboxylic acid (2,5-difluorophenyl)amide;
6-[4-(2-Trifluoromethylbenzoyl)piperazin-1-yl]-pyridazine-3-carboxylic acid (2,6-dichlorophenyl)amide;
6-[4-(2-Trifluoromethylbenzoyl)piperazin-1-yl]-pyridazine-3-carboxylic acid (2-trifluoromethylphenyl)amide;
6-[4-(2-Trifluoromethylbenzoyl)piperazin-1-yl]-pyridazine-3-carboxylic acid (4-trifluoromethylphenyl)amide;
6-[4-(2-Trifluoromethylbenzoyl)-piperazin-1-yl]-pyridazine-3-carboxylic acid (3-trifluoromethylphenyl)amide;
6-[4-(2-Trifluoromethylbenzoyl)piperazin-1-yl]-pyridazine-3-carboxylic acid phenylamide;
6-[4-(2-Trifluoromethylbenzoyl)piperazin-1-yl]-pyridazine-3-carboxylic acid (5-chloro-2-methoxyphenyl)amide;
6-[4-(2-Trifluoromethylbenzoyl)piperazin-1-yl]-pyridazine-3-carboxylic acid (2,5-dimethoxyphenyl)amide;
6-[4-(2-Trifluoromethylbenzoyl)piperazin-1-yl]-pyridazine-3-carboxylic acid (2-chloro-4-methoxyphenyl)amide;
6-[4-(2-Trifluoromethylbenzoyl)-piperazin-1-yl]-pyridazine-3-carboxylic acid (4-methoxyphenyl)amide;
6-[4-(2-Trifluoromethylbenzoyl)piperazin-1-yl]-pyridazine-3-carboxylic acid (2-methoxyphenyl)amide;
6-[4-(2-Trifluoromethylbenzoyl)piperazin-1-yl]-pyridazine-3-carboxylic acid (3-methoxyphenyl)amide;
4-({6-[4-(2-Trifluoromethylbenzoyl)piperazin-1-yl]-pyridazine-3-carbonyl}amino)-benzoic acid methyl ester;
4-({6-[4-(2-Trifluoromethylbenzoyl)piperazin-1-yl]-pyridazine-3-carbonyl}amino)-benzoic acid;
2-({6-[4-(2-Trifluoromethylbenzoyl)piperazin-1-yl]-pyridazine-3-carbonyl}amino)-benzoic acid methyl ester;
2-({6-[4-(2-Trifluoromethylbenzoyl)piperazin-1-yl]-pyridazine-3-carbonyl}amino)-benzoic acid;
6-[4-(2-Trifluoromethylbenzoyl)piperazin-1-yl]-pyridazine-3-carboxylic acid (3,4-dichlorophenyl)amide;
6-[4-(2-Trifluoromethylbenzoyl)piperazin-1-yl]-pyridazine-3-carboxylic acid [2-(2,4-fluorophenyl)ethyl]amide;
6-[4-(2-Trifluoromethylbenz-oyl)piperazin-1-yl]-pyridazine-3-carboxylic acid [2-(2-fluorophenyl)ethyl]amide;
6-[4-(2-Trifluoromethylbenzoyl)piperazin-1-yl]-pyridazine-3-carboxyli-c acid [2-(4-chlorophenyl)ethyl]amide;
6-[4-(2-Trifluoromethylbenzoyl)piperazin-1-yl]-pyridazine-3-carboxylic acid [2-(3-chlorophenyl)ethyl]amide;
6-[4-(2-Trifluoromethylbenzoyl)piperazin-1-yl]-pyridazine-3-carboxylic acid (2-phenylpropyl)amide;
6-[4-(2-Trifluoromethylbenzoyl)piperazin-1-yl]-pyridazine-3-carboxylic acid (2-biphenyl-4-ylethyl)amide;
(R)-6-[4-(2-Trifluoromethylbenzoyl)piperazin-1-yl]-pyridazine-3-carboxylic acid (2-hydroxy-2-phenylethyl)amide;
(S)-6-[4-(2-Trifluoromethylbenzoyl)-piperazin-1-yl]-pyridazine-3-carboxylic acid (2-hydroxy-2-phenylethyl)amide;
Acetic acid 1-phenyl-2-({6-[4-(2-trifluoromethyl-benzoyl)-piperazin-1-yl]-pyridazine-3-carbonyl}amino)ethyl ester;
6-[4-(2-Trifluoromethylbenzoyl)piperazin-1-yl]-pyridazine-3-carboxylic acid [3-(4-fluorophenyl)propyl]amide;
6-[4-(2-Trifluoromethylbenzoyl)piperazin-1-yl]-pyridazine-3-carboxylic acid (2,2-difluoro-2-phenylethyl)amide;
6-[4-(2-Trifluoromethylbenzoyl)piperazin-1-yl]-pyridazine-3-carboxylic acid [2-(3-fluorophenyl)-2-hydroxyethyl]amide;
6-[4-(2-Trifluoromethylbenzoyl)piperazin-1-yl]-pyridazine-3-carboxylic acid (4-hydroxybutyl)amide;
6-[4-(2-Trifluoromethylbenzoyl)piperazin-1-yl]-pyridazine-3-carboxylic acid (3-hydroxy-4,4-dimethylpentyl)amide;
6-[4-(2-Trifluoromethylbenzoyl)piperazin-1-yl]-pyridazine-3-carboxylic acid (3-hydroxy-3-methylbutyl)amide;
6-[4-(2-Trifluoromethylbenzoyl)piperazin-1-yl]-pyridazine-3-carboxylic acid (2-hydroxy-3,3-dimethylbutyl)amide;
6-[4-(5-Fluoro-2-trifluoromethylbenzoyl)piperazin-1-yl]-pyridazine-3-carboxylic acid (2-hydroxy-3,3-dimethylbutyl)amide;
6-[4-(2-Nitrobenzoyl)piperazin-1-yl]-pyridazine-3-carboxylic acid (3-methylbutyl)amide;
6-[4-(2-Chlorobenzoyl)piperazin-1-yl]-pyridazine-3-carboxylic acid (3-methylbutyl)amide;
6-[4-(2,4-Dichlorobenzoyl)piperazin-1-yl]-pyridazine-3-carboxylic acid (3-methyl butyl)amide;
6-[4-(2-Aminobenzoyl)piperazin-1-yl]-pyridazine-3-carboxylic acid (3-methylbutyl)amide;
6-[4-(2-Trifluoromethylbenzoyl)piperazin-1-yl]-pyridazine-3-carboxylic acid [2-(4-chlorophenoxy)ethyl]amide;
6-[4-(2-Trifluoromethylbenzoyl)piperazin-1-yl]-pyridazine-3-carboxylic acid [2-(4-fluorophenoxy)ethyl]amide;
6-[4-(2-Trifluoromethyl benzoyl)piperazin-1-yl]-pyridazine-3-carboxylic acid (3,3-dimethylbutyl)amide;
6-[4-(2-Trifluoromethylbenzoyl)piperazin-1-yl]-pyridazine-3-carboxylic acid pentylamide;
4-({6-[4-(2-Trifluoromethylbenzoyl)piperazin-1-yl]-pyridazine-3-carbonyl}amino)butyric acid ethyl ester;
6-[4-(5-Fluoro-2-trifluoromethylbenzoyl)piperazin-1-yl]-pyridazine-3-carboxylic acid pentylamide;
6-[4-(2-Trifluoromethyl benzoyl)piperazin-1-yl]-pyridazine-3-carboxylic acid (4-methylpentyl)amide;
6-[4-(2-Fluoro-6-trifluoromethylbenzoyl)piperazin-1-yl]-pyridazine-3-carboxylic acid (3-methylbutyl)amide;
6-[4-(2,6-Difluorobenzoyl)piperazin-1-yl]-pyridazine-3-carboxylic acid (3-methylbutyl)amide;
6-[4-(2-Trifluoromethylbenzoyl)piperazin-1-yl]-pyridazine-3-carboxylic acid (2-oxo-2-phenylethyl)amide;
Acetic acid 1,1-dimethyl-3-({6-[4-(2-trifluoromethyl-benzoyl)piperazin-1-yl]-pyridazine-3-carbonyl}amino)propyl ester;
6-[4-(2-Trifluoromethylbenzoyl)piperazin-1-yl]-pyridazine-3-carboxylic acid (2-phenoxyethyl)amide;
6-[4-(2-Trifluoromethylbenzoyl)piperazin-1-yl]-pyridazine-3-carboxylic acid hexylamide;
6-[4-(2-Trifluoromethylbenzoyl)piperazin-1-yl]-pyridazine-3-carboxylic acid (4-methylpentyl)amide;
6-[4-(5-Fluoro-2-trifluoromethylbenzoyl)piperazin-1-yl]-pyridazine-3-carboxylic acid (4-methylpentyl)amide;
6-[2,5-Dimethyl-4-(2-trifluoromethylbenzoyl)piperazin-1-yl]-pyridazine-3-carboxylic acid pentylamide;
6-[4-(2-Trifluoromethylbenzoyl)-piperazin-1-yl]-pyridazine-3-carboxylic acid heptylamide;
6-[4-(2-Sulfamoylbenzoyl)-piperazin-1-yl]-pyridazine-3-carboxylic acid (3-methylbutyl)-amide;
6-[4-(5-Chloro-2-trifluoromethyl-benzoyl)-piperazin-1-yl]-pyridazine-3-carboxylic acid hexylamide;
6-[4-(2-Trifluoromethylbenzoyl)-piperazin-1-yl]-pyridazine-3-carboxylic acid (2-cyclopropyl-2-oxoethyl)amide;
4-Trifluoromethyl-6-[4-(2-trifluoromethylbenzoyl)-piperazin-1-yl]-pyridazine-3-carboxylic acid (3-methyl-butyl)amide;
6-[4-(5-Fluoro-2-trifluoromethylbenzoyl)piperazin-1-yl]-pyridazine-3-carboxylic acid pentyl-4-enylamide;
6-(4-Benzoylpiperazin-1-yl)-pyridazine-3-carboxylic acid (2-cyclopropylethyl)amide;
6-[4-(2-Chloro-5-fluorobenzoyl)piperazin-1-yl]-pyridazine-3-carboxylic acid (2-cyclopropylethyl)amide;
6-[4-(5-Chloro-2-trifluoromethylbenzoyl)piperazin-1-yl]-pyridazine-3-carboxylic acid (2-cyclopropylethyl)amide;
6-[4-(2,6-Difluorobenzoyl)piperazin-1-yl]-pyridazine-3-carboxylic acid (2-cyclopropylethyl)amide;
6-[4-(2,5-Bis-trifluoromethylbenzoyl)piperazin-1-yl]-pyridazine-3-carboxylic acid (2-cyclopropylethyl)amide;
6-[4-(2,4-Bis-trifluoromethylbenzoyl)piperazin-1-yl]-pyridazine-3-carboxylic acid (2-cyclopropylethyl)amide;
6-[4-(2,5-Difluorobenzoyl)piperazin-1-yl]-pyridazine-3-carboxylic acid (2-cyclopropylethyl)amide;
6-[4-(5-Fluoro-2-trifluoromethylbenzoyl)piperazin-1-yl]-pyridazine-3-carboxylic acid (3-cyclopropylpropyl)amide;
6-[4-(2-Fluorobenzoyl)piperazin-1-yl]-pyridazine-3-carboxylic acid (2-cyclopropylethyl)amide;
6-[4-(3-Fluoro-2-trifluoromethylbenzoyl)piperazin-1-yl]-pyridazine-3-carboxylic acid (2-cyclopropylethyl)amide;
6-[4-(4-Fluoro-2-trifluoromethylbenzoyl)piperazin-1-yl]-pyridazine-3-carboxylic acid (3-cyclopropylpropyl)amide;
6-[4-(2-Trifluoromethylbenzoyl)piperazin-1-yl]-pyridazine-3-carboxylic acid (2-methylcyclopropylmethyl)amide;
6-[4-(5-Fluoro-2-methoxybenzoyl)piperazin-1-yl]-pyridazine-3-carboxylic acid (2-cyclopropylethyl)amide;
6-[4-(2-Dimethylaminobenzoyl)piperazin-1-yl]-pyridazine-3-carboxylic acid (2-cyclopropylethyl)amide;
6-[4-(2-Chloro-5-dimethylaminobenzoyl)piperazin-1-yl]-pyridazine-3-carboxylic acid (2-cyclopropylethyl)amide;
6-[4-(2,5-Dimethylbenzoyl)piperazin-1-yl]-pyridazine-3-carboxylic acid (2-cyclopropylethyl)amide;
6-[4-(2,5-Dichlorobenzoyl)piperazin-1-yl]-pyridazine-3-carboxylic acid (2-cyclopropylethyl)amide;
6-[4-(2-Trifluoromethylbenzoyl)piperazin-1-yl]-pyridazine-3-carboxylic acid cyclobutylmethylamide;
Acetic acid 2-{4-[6-(2-cyclopropylethylcarbamoyl)-pyridazin-3-yl]-piperazine-1-carbonyl}phenyl ester;
6-[4-(2-Trifluoromethylbenzoyl)piperazin-1-yl]-pyridazine-3-carboxylic acid (2-cyclopropyl-2-hydroxyethyl)amide;
6-[4-(2-Trifluoromethylbenzoyl)piperazin-1-yl]-pyridazine-3-carboxylic acid (2-phenylcyclopropylmethyl)amide;
6-[4-(2-Trifluoromethylbenzoyl)piperazin-1-yl]-pyridazine-3-carboxylic acid (3-cyclopropylpropyl)amide;
6-[4-(2-Cyanobenzoyl)piperazin-1-yl]-pyridazine-3-carboxylic acid (2-cyclopropylethyl)amide;
6-{4-[2-(2-Trifluoromethyl phenyl)acetyl]-piperazin-1-yl}pyridazine-3-carboxylic acid (2-cyclopropylethyl)amide;
6-[4-(4-Fluoro-2-trifluoromethylbenzoyl)piperazin-1-yl]-pyridazine-3-carboxylic acid (2-cyclopropylethyl)amide;
6-[4-(5-Chloro-2-trifluoromethylbenzoyl)piperazin-1-yl]-pyridazine-3-carboxylic acid (3-cyclopropylpropyl)amide;
6-[3,5-Dimethyl-4-(2-trifluoromethylbenzoyl)piperazin-1-yl]-pyridazine-3-carboxylic acid (2-cyclopropylethyl)amide;
2-{4-[6-(2-Cyclopropylethylcarbamoyl)pyridazin-3-yl]-piperazine-1-carbonyl}benzoic acid methyl ester;
6-[4-(2-Trifluoromethyl-benzoyl)-piperazin-1-yl]-pyridazine-3-carboxylic acid (2-cyclobutylethyl)amide;
2-{4-[6-(2-Cyclopropyl-ethylcarbamoyl)-pyridazin-3-yl]-piperazine-1-carbonyl}-benzoic acid;
6-[4-(5-Chloro-2-trifluoromethyl-benzoyl)-piperazin-1-yl]-pyridazine-3-carboxylic acid (2-cyclobutylethyl)amide;
6-[4-(5-Fluoro-2-trifluoromethylbenzoyl)-piperazin-1-yl]-pyridazine-3-carboxylic acid (2-cyclobutylethyl)amide;
6-[4-(2-Trifluoromethylbenzoyl)-piperazin-1-yl]-pyridazine-3-carboxylic acid (3-cyclobutyl-propyl)amide;
6-[4-(5-Fluoro-2-trifluoromethylbenzoyl)-piperazin-1-yl]-pyridazine-3-carboxylic acid (2-cyclopropylethyl)amide;
6-[4-(2-Trifluoromethyl-thiobenzoyl)-piperazin-1-yl]-pyridazine-3-carboxylic acid (2-cyclopropylethyl)amide;
6-[4-(2-Trifluoromethyl benzoyl)-piperazin-1-yl]-pyridazine-3-carboxylic acid (4-cyclopropylbutyl)amide;
6-[4-(2-Trifluoromethylbenzoyl)-piperazin-1-yl]-pyridazine-3-carboxylic acid (2,2-dimethyl-cyclopropylmethyl)amide;
6-[4-(Pyridine-2-carbonyl)piperazin-1-yl]-pyridazine-3-carboxylic acid (2-cyclopropylethyl)amide;
6-[4-(2-Ttrifluoromethylfuran-3-carbonyl)piperazin-1-yl]-pyridazine-3-carboxylic acid (2-cyclopropylethyl)amide;
6-[4-(2-Chloro-4-trifluoromethylpyrimidine-5-carbonyl)piperazin-1-yl]-pyridazine-3-carboxylic acid (2-cyclopropylethyl)amide;
6-[4-(5-Methyl-2-trifluoromethylfuran-3-carbonyl)piperazin-1-yl]-pyridazine-3-carboxylic acid (2-cyclopropylethyl)amide;
6-[4-(2-Chloropyridine-3-carbonyl)piperazin-1-yl]-pyridazine-3-carboxylic acid (2-cyclopropylethyl)amide;
6-[4-(2-Methyl-5-trifluoromethyloxazole-4-carbonyl)piperazin-1-yl]-pyridazine-3-carboxylic acid (2-cyclopropylethyl)amide;
6-[4-(2,6-Dichloropyridine-3-carbonyl)piperazin-1-yl]-pyridazine-3-carboxylic acid (2-cyclopropylethyl)amide;
6-[4-(Pyrrolidine-1-carbonyl)piperazin-1-yl]-pyridazine-3-carboxylic acid (2-cyclopropylethyl)amide;
6-[4-(1-Methyl-1H-pyrrole-2-carbonyl)piperazin-1-yl]-pyridazine-3-carboxylic acid (2-cyclopropylethyl)amide;
6-[4-(Tetrahydrofuran-2-carbonyl)piperazin-1-yl]-pyridazine-3-carboxylic acid (2-cyclopropylethyl)amide;
6-{4-[2-(2-Trifluoromethylphenyl)acetyl]piperazin-1-yl}-pyridazine-3-carboxylic acid (2-cyclopropylethyl)amide;
4-[6-(3-Methylbutylcarbamoyl)pyridazin-3-yl]-piperazine-1-carboxylic acid t-butyl ester;
4-[6-(2-Cyclopropylethylcarbamoyl)pyridazin-3-yl]-piperazine-1-carboxylic acid t-butyl ester;
6-[4-(4,4,4-Trifluoro-3-hydroxy-3-trifluoromethylbutyryl)piperazin-1-yl]-pyridazine-3-carboxylic acid (2-cyclopropylethyl)amide;
6-[4-(4,4,4-Trifluoro-3-hydroxy-3-methylbutyryl)piperazin-1-yl]-pyridazine-3-carboxylic acid (2-cyclopropylethyl)amide;
and 6-[4-(3,3,3-Trifluoro-2-hydroxy-2-methylpropionyl)piperazin-1-yl]-pyridazine-3-carboxylic acid (2-cyclopropylethyl)amide;
6-[4-(1-Hydroxycyclopropanecarbonyl)piperazin-1-yl]-pyridazine-3-carboxylic acid (2-cyclopropylethyl)amide;
6-(4-Cyclobutanecarbonylpiperazin-1-yl)pyridazine-3-carboxylic acid (2-cyclopropylethyl)amide;
6-[4-(2-Trifluoromethylcyclopropanecarbonyl)piperazin-1-yl]-pyridazine-3-carboxylic acid (2-cyclopropylethyl)amide;
6-(4-Cyclohexanecarbonylpiperazin-1-yl)pyridazine-3-carboxylic acid (2-cyclopropylethyl)amide;
6-[4-(2-Methylcyclohexanecarbonyl)piperazin-1-yl]-pyridazine-3-carboxylic acid (2-cyclopropylethyl)amide;
6-[4-(3-Methylcyclohexanecarbonyl)piperazin-1-yl]-pyridazine-3-carboxylic acid (2-cyclopropylethyl)amide;
6-[4-(4-Methylcyclohexanecarbonyl)piperazin-1-yl]-pyridazine-3-carboxylic acid (2-cyclopropylethyl)amide;
6-[4-(2-Methylcyclopropanecarbonyl)piperazin-1-yl]-pyridazine-3-carboxylic acid (2-cyclopropylethyl)amide;
6-[4-(2,2,3,3-Tetramethylcyclopropanecarbonyl)piperazin-1-yl]pyridazine-3-carboxylic acid (2-cyclopropylethyl)amide;
6-[4-(2-Ethylbutyryl)piperazin-1-yl]-pyridazine-3-carboxylic acid (2-cyclopropylethyl)amide;
6-[4-(3,3,3-Trifluoro-2-methyl-2-trifluoromethylpropionyl)piperazin-1-yl]-pyridazine-3-carboxylic acid (2-cyclopropylethyl)amide;
6-[4-(2,2-Dimethylpropionyl)piperazin-1-yl]-pyridazine-3-carboxylic acid (2-cyclopropylethyl)amide;
6-[4-(2,2-Dimethylbutyryl)piperazin-1-yl]-pyridazine-3-carboxylic acid (2-cyclopropylethyl)amide;
6-[4-(2,2-Dimethylpentanoyl)piperazin-1-yl]-pyridazine-3-carboxylic acid (2-cyclopropylethyl)amide;
6-[4-(4,4,4-Trifluorobut-2-enoyl)piperazin-1-yl]-pyridazine-3-carboxylic acid (2-cyclopropylethyl)amide; and
6-[4-(4,4,4-Trifluoro-3-trifluoromethylbut-2-enoyl)-piperazin-1-yl]-pyridazine-3-carboxylic acid (2-cyclopropyl-ethyl)amide;
or a pharmaceutically acceptable salt thereof.

18. A pharmaceutical composition according to claim 11, wherein an inhibitor of protein tyrosine phosphatase (PTPase) is a compound of the formula 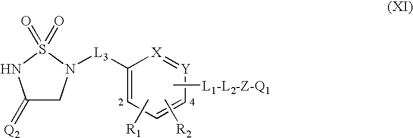
wherein
R₁ is hydrogen, halogen, hydroxy, alkoxy, carboxy, cyano, nitro, trifluoromethyl, alkynyl, alkylthio, heteroaralkyl, heteroaralkoxy or heteroaryloxy provided that R₁ is located at the 2-position when L₃ is —(CHR)ₛ— in which s is zero; or
R₁ is optionally substituted alkyl, alkenyl, optionally substituted amino, aralkyl, aralkoxy, aralkylthio, aryloxy, arylthio or cycloalkyl provided that a monocyclic aryl group which is substituted at the para position with a methylene or ethylene bridged nitrogen containing heterocycle does not constitute part of R₁ when
(i) R₁ is located at the 2-position and L₃ is —(CHR)ₛ— in which s is zero;
(ii) X and Y each are CH; and
(iii) Q₂ is oxygen; or
C—R₁ may be replaced with nitrogen or N→O; or
R₁ and R₂ combined together with the carbon atoms to which R₁ and R₂ are attached form an optionally substituted fused 5- to 6-membered aromatic or heteroaromatic ring provided that R₁ and R₂ are attached to carbon atoms adjacent to each other; or
R₂ is hydrogen, halogen, hydroxy, alkoxy, cyano, trifluoromethyl, nitro, optionally substituted amino, optionally substituted alkyl, alkylthio, aralkyl, heteroaralkyl, aralkoxy, heteroaralkoxy, aralkylthio, aryloxy, heteroaryloxy, arylthio or cycloalkyl; or
R₂ is —C(O)R₃ wherein
R₃ is hydroxy or optionally substituted alkoxy; or
R₃ is —NR₄R₅ in which R₄ and R₅ are independently hydrogen, optionally substituted alkyl, alkenyl, alkynyl, cycloalkyl, aryl, heterocyclyl, aralkyl or heteroaralkyl;
L₁ is a single bond; or
L₁ is carbon which combined together with R₂ and the carbon atoms to which L₁ and R₂ are attached form an optionally substituted fused 5- or 6-membered aromatic or heteroaromatic ring provided that L₁ and R₂ are attached to carbon atoms adjacent to each other; or
L₁ is CH or nitrogen which taken together with R₂ and the carbon atoms to which L₁ and R₂ are attached form a fused 5- to 7-membered ring which may be interrupted with one or two heteroatoms selected from oxygen, nitrogen and sulfur provided that L₁ and R₂ are attached to carbon atoms adjacent to each other; or
L₁ is CH, oxygen, sulfur or nitrogen and L₂ is carbon which combined together with L₁, R₂ and the carbon atoms to which L₁ and R₂ are attached form an optionally substituted fused 5- or 6-membered aromatic or heteroaromatic ring provided that L₁ and R₂ are attached to carbon atoms adjacent to each other; or
L₁ is —CH₂—, oxygen, sulfur or —NR₆— and L₂ is CH which taken together with L₁, R₂ and the carbon atoms to which L₁ and R₂ are attached form a fused 5- to 7-membered ring which may be interrupted with one or two heteroatoms selected from oxygen, nitrogen and sulfur wherein
R₆ is hydrogen, optionally substituted alkyl, aralkyl, heteroaralkyl, alkoxycarbonyl, aryloxycarbonyl, carbamoyl, sulfonyl or acyl provided that L₁ and R₂ are attached to carbon atoms adjacent to each other;
L₂ is —(CHR₇)ₙ— wherein
R₇ is hydrogen, hydroxy, alkoxy, carboxy, optionally substituted alkyl, cycloalkyl, aryl or heteroaryl;
n is zero or an integer from 1 to 4;
Z is —(CHR₈)ₘ—, —(CH₂)ₘO(CHR₈)ᵣ—, —(CH₂)ₘS(CHR₈)ᵣ— or —(CH₂)ₘNR₉(CHR₈)ᵣ— wherein
R₈ is hydrogen, optionally substituted alkyl, cycloalkyl, aryl or heterocyclyl;
R₉ is hydrogen, optionally substituted alkyl, cycloalkyl, aryl, heterocyclyl, aralkyl, heteroaralkyl, alkoxycarbonyl, aryloxycarbonyl, heteroaryloxycarbonyl, carbamoyl, sulfonyl, acyl or acylamino;
m and r are independently zero or an integer of 1 or 2;
Q₁ is hydrogen, optionally substituted alkyl, cycloalkyl, aryl or heterocyclyl provided that
(i) Q₁ is not 2-phenyloxazol-4-yl when
R₁ and R₂ are hydrogen;
X and Y each are CH;
L₁ is a single bond located at the 4-position;
L₂ is —(CHR₇)ₙ— wherein n is zero;
L₃ is —(CHR)ₛ— wherein s is zero;
Z is —(CH₂)ₘO(CHR₈)ᵣ— wherein R₈ is hydrogen, m is zero and r is 2; and
Q₂ is oxygen; or
(ii) Q₁ is not hydrogen when
R₁ and R₂ are hydrogen;
X and Y each are CH;
L₁ is a single bond;
L₂ is —(CHR₇)ₙ— wherein n is zero;
L₃ is —(CHR)ₛ— wherein R is hydrogen and s is 1;
Z is —(CHR₈)ₘ— wherein m is zero; and
Q₂ is oxygen; or
Q₁ is —C(O)NR₄R₅ₐ, —C(O)R₁₀, —C(O)OR₁₀ or —S(O)_{q}R₁₀ wherein R₄ₐ and R₅ₐ are as defined for R₄ and R₅; R₁₀ is optionally substituted alkyl, cycloalkyl, aryl, heterocyclyl, aralkyl or heteroaralkyl; q is an integer of 1 or 2; or
Q₁ is a radical of the formula 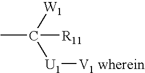
W₁ is aryl, heteroaryl, aralkyl or heteroaralkyl; or
W₁ is —C(O)R₃ₐ in which R₃ₐ is hydroxy or optionally substituted alkoxy; or
R₃ₐ is —NR₄ₐR₅ₐ in which R₄ₐ and R₅ₐ are as defined for R₄ and R₅;
R₁₁ is hydrogen, alkyl or aryl;
U₁ is —C(O)—, —S(O)₂— or —(CH₂)ᵣ— in which r is as defined for Z;
V₁ is hydroxy, alkoxy, aryl, heteroaryl, optionally substituted alkyl or cycloalkyl; or
V₁ is —NR_{4b}R_{5b} in which R_{4b} and R_{5b} are as defined for R₄ and R₅ provided that
(i) L₂ is —(CHR₇)ₙ— in which n is an integer of 1 or 2; and
(ii) Z is —(CHR₈)ₘ— in which m is zero; or
Q₁ is a radical of the formula 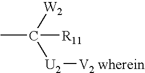
W₂ is —C(O)R₃ₐ in which R₃ₐ is hydroxy or optionally substituted alkoxy; or
R₃ₐ is —NR₄ₐR₅ₐ in which R₄ₐ and R₅ₐ are as defined for R₄ and R₅;
R₁₁ is hydrogen, alkyl or aryl;
U₂ is —(CH₂)ₚ— in which p is zero or 1;
V₂ is —NR_{4b}C(O)R_{5b}, —NR_{4b}C(O)OR_{5b}, —NR_{4b}C(O)NR_{4c}R_{5b} or —NR_{4b}S(O)₂R_{5b} in which
R_{4b} and R_{4c} are as defined for R₄, and R_{5b} has a meaning as defined for R₅ provided that
(i) L₂ is —(CHR₇)ₙ— in which n is an integer of 1 or 2; and
(ii) Z is —(CHR₈)ₘ— in which m is zero; or
Q₁ is a radical of the formula 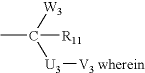
W₃ is —C(O)R₃ₐ in which R₃ₐ is hydroxy or optionally substituted alkoxy; or
R₃ₐ is —NR₄ₐR₅ₐ in which R₄ₐ and R₅ₐ are as defined for R₄ and R₅;
R₁₁ is hydrogen, alkyl or aryl;
U₃ is —(CH₂)ₚ— in which p is zero or 1;
V₃ is —NHC(O)CHR_{4b}NHC(O)R₁₂ wherein R_{4b} is as defined for R₄; R₁₂ is hydrogen, aryl, heterocyclyl, aralkyl, heteroaralkyl, optionally substituted alkyl, alkoxy or cycloalkyl; or
R₁₂ is —NR_{4c}R_{5b}, in which R_{4c} and R_{5b} are as defined for R₄ and R₅ provided that
(i) L₂ is —(CHR₇)ₙ— in which n is an integer of 1 or 2; and
(ii) Z is —(CHR₈)ₘ— in which m is zero;
L₃ is —(CHR)ₛ— wherein
R is hydrogen, carboxy, optionally substituted alkyl, cycloalkyl, aryl or heteroaryl;
s is zero or an integer from 1 to 3;
Q₂ is oxygen, sulfur or NR₁₃ wherein
R₁₃ is hydrogen, hydroxy or lower alkyl;
X and Y are independently CH or nitrogen; or
—X═Y— is sulfur, oxygen or —NR₁₄— wherein
R₁₄ is hydrogen, optionally substituted alkyl, alkoxycarbonyl, acyl, aryloxycarbonyl, heteroaryloxycarbonyl, carbamoyl or sulfonyl;
or a pharmaceutically acceptable salt thereof; or a prodrug derivative thereof.

19. A pharmaceutical composition according to claim 11 for the prevention of, delay the onset of or treatment of a disease or a condition modulated by the inhibition of renin activity and/or insulin secretion enhancer and/or insulin sensitizer.

20. A pharmaceutical composition according to claim 19, wherein a disease or a condition modulated by the inhibition of renin activity and/or insulin secretion enhancer and/or insulin sensitizer is selected from the group consisting of hypertension, congestive heart failure, diabetes, especially type 2 diabetes mellitus, mature onset diabetes of the young (MODY), diabetic retinopathy, macular degeneration, diabetic nephropathy, hypertensive or non-hypertensive nephropathy, IgA nephropathy, glomerulosclerosis, chronic renal failure, diabetic neuropathy, metabolic syndrome, cardiac syndrome X, atherosclerosis, coronary heart disease, angina pectoris, myocardial infarction, stroke, coronary and vascular restenosis, hyperglycemia, hyperinsulinaemia, hyperlipidaemia, hypertryglyceridemia, insulin resistance, impaired glucose metabolism (IGM), conditions of impaired glucose tolerance (IGT), IGM and/or IGT or increased inflammation in women with polycystic ovary syndrome or women with prior gestational diabetes, conditions of impaired fasting plasma glucose, obesity, diabetic retinopathy, macular degeneration, cataracts, foot ulcerations, endothelial dysfunction, impaired vascular compliance and obstructive sleep apnea. Preferably, the said combination may be used for the treatment of hypertension, including ISH, as well as congestive heart failure, metabolic syndrome, endothelial dysfunction, impaired vascular compliance, IGT, diabetes especially type II diabetes mellitus, hypertensive or non-hypertensive nephropathy, IgA nephropathy, as well as retardation or prolongation of the progression of prediabetes to diabetes.

21. A method for the prevention of, delay the onset of or treatment of a disease or a condition modulated by the inhibition of renin activity and/or insulin secretion enhancer and/or insulin sensitizer, which method comprises administering to a warm-blooded animal, including man, in need thereof, a therapeutically effective amount of a combination comprising a renin inhibitor, or a pharmaceutically acceptable salt thereof, and at least one therapeutic agent selected from the group consisting of
(a) an insulin secretion enhancer, or a pharmaceutically acceptable salt thereof; and
(b) an insulin sensitizer, or a pharmaceutically acceptable salt thereof.

22. A method according to claim 21, wherein a disease or a condition modulated by the inhibition of renin activity and/or insulin secretion enhancer and/or insulin sensitizer is selected from the group consisting of hypertension, congestive heart failure, diabetes, especially type 2 diabetes mellitus, mature onset diabetes of the young (MODY), diabetic retinopathy, macular degeneration, diabetic nephropathy, hypertensive or non-hypertensive nephropathy, IgA nephropathy, glomerulosclerosis, chronic renal failure, diabetic neuropathy, metabolic syndrome, cardiac syndrome X, atherosclerosis, coronary heart disease, angina pectoris, myocardial infarction, stroke, coronary and vascular restenosis, hyperglycemia, hyperinsulinaemia, hyperlipidaemia, hypertryglyceridemia, insulin resistance, impaired glucose metabolism (IGM), conditions of impaired glucose tolerance (IGT), IGM and/or IGT or increased inflammation in women with polycystic ovary syndrome or women with prior gestational diabetes, conditions of impaired fasting plasma glucose, obesity, diabetic retinopathy, macular degeneration, cataracts, foot ulcerations, endothelial dysfunction, impaired vascular compliance and obstructive sleep apnea. Preferably, the said combination may be used for the treatment of hypertension, including ISH, as well as congestive heart failure, metabolic syndrome, endothelial dysfunction, impaired vascular compliance, IGT, diabetes especially type II diabetes mellitus, hypertensive or non-hypertensive nephropathy, IgA nephropathy, as well as retardation or prolongation of the progression of prediabetes to diabetes.

23. A method according to claim 22, wherein a renin inhibitor is selected from the group consisting of RO 66-1132, RO 66-1168 and a compound of the formula 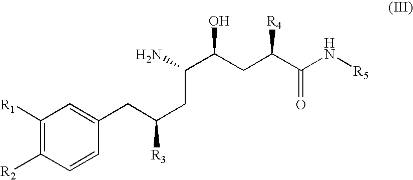
wherein R₁ is halogen, C₁₋₆halogenalkyl, C₁₋₆alkoxy-C₁₋₆alkyloxy or C₁₋₆alkoxy-C₁₋₆alkyl; R₂ is halogen, C₁₋₄alkyl or C₁₋₄alkoxy; R₃ and R₄ are independently branched C₃₋₆alkyl; and R₅ is cycloalkyl, C₁₋₆alkyl, C₁₋₆hydroxyalkyl, C₁₋₆alkoxy-C₁₋₆alkyl, C₁₋₆alkanoyloxy-C₁₋₆alkyl, C₁₋₆aminoalkyl, C₁₋₆alkylamino-C₁₋₆alkyl, C₁₋₆dialkylamino-C₁₋₆alkyl, C₁₋₆alkanoylamino-C₁₋₆alkyl, HO(O)C—C₁₋₆alkyl, C₁₋₆alkyl-O—(O)C—C₁₋₆alkyl, H₂N—C(O)—C₁₋₆alkyl, C₁₋₆alkyl-HN—C(O)—C₁₋₆alkyl or (C₁₋₆alkyl)₂N—C(O)—C₁₋₆alkyl; or a pharmaceutically acceptable salt thereof.

24. A method according to claim 23, wherein a renin inhibitor is a compound of formula (III) having the formula 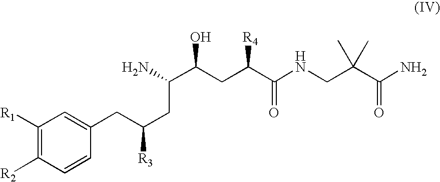
wherein R₁ is 3-methoxypropyloxy; R₂ is methoxy; and R₃ and R₄ are isopropyl; or a pharmaceutically acceptable salt thereof.

25. A method according to claim 24, wherein the compound of formula (IV) is in the form of the hemi-fumarate salt thereof.

26. A method according to claim 22, wherein a glucokinase activator is selected from the group consisting of 6-[(3-isobutoxy-5-isopropoxybenzoyl)amino]nicotinic acid (GKA1), 5-({3-isopropoxy-5-[2-(3-thienyl)ethoxy]benzoyl}amino)-1,3,4-thiadiazole-2-carboxylicacid (GKA2), 2-(S)-Cyclohexyl-1-(R)-(4-methanesulfonyl-phenyl)-cyclopropanecarboxylic acid thiazol-2-ylamide (LY2121260) and RO-28-1675 or a pharmaceutically acceptable salt thereof.

27. A method according to claim 22, wherein a glucokinase activator is a compound of the formula
R—NH-Q (IX)
wherein
(i) Q is a 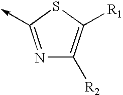
radical in which R₁ and R₂ are independently hydrogen or halogen; or
Q is a 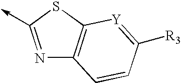
radical in which R₃ is hydrogen, halogen, alkyl, cycloalkyl, aryl, alkoxy, cycloalkoxy, aryloxy, alkylthio, cycloalkylthio, arylthio, acyl, sulfonyl, alkylamino, cycloalkylamino, arylamino, acylamino, sulfonamido or alkoxycarbonyl; Y is CH or nitrogen; and
R is a radical of the formula 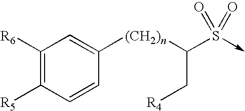
wherein
R₄ is C₂₋₄alkyl, C₃₋₇cycloalkyl or C₅₋₇heterocycloalkyl;
R₅ and R₆ are independently hydrogen, halogen, cyano, R₇, —C(O)R₇ or —S(O)₂R₇ wherein
R₇ is —(CR₈R₉)ₘ—W—R₁₀ in which
R₈ and R₉ are independently hydrogen or lower alkyl;
W is a bond, O, S or —NR₁₁ in which
R₁₁ is hydrogen or lower alkyl;
R₁₀ is hydrogen, alkyl, cycloalkyl, aryl or heterocyclyl; or R₁₀ and R₁₁, combined, are alkylene which together with the nitrogen atom to which they are attached form a 5- to 7-membered ring;
m is zero or an integer from 1 to 5;
n is zero or an integer of 1 or 2;
or an optical isomer thereof; or a pharmaceutically acceptable salt thereof; or
(ii) Q is a 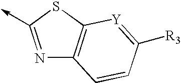
radical in which R₃ is hydrogen, halogen, alkyl, cycloalkyl, aryl, alkoxy, cycloalkoxy, aryloxy, alkylthio, cycloalkylthio, arylthio, acyl, sulfonyl, alkylamino, cycloalkylamino, arylamino, acylamino, sulfonamido or alkoxycarbonyl; and
R is a radical of the formula 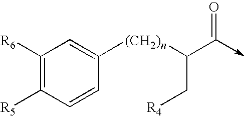
wherein
R₄ is C₂₋₄alkyl, C₃₋₇cycloalkyl or C₅₋₇heterocycloalkyl;
R₅ and R₆ are independently hydrogen, halogen, cyano, R₇, —C(O)R₇ or —S(O)₂R₇ wherein
R₇ is —(CR₈R₉)ₘ—W—R₁₀ in which
R₈ and R₉ are independently hydrogen or lower alkyl;
W is a bond, O, S or —NR₁₁ in which
R₁₁ is hydrogen or lower alkyl;
R₁₀ is hydrogen, alkyl, cycloalkyl, aryl or heterocyclyl; or R₁₀ and R₁₁, combined, are alkylene which together with the nitrogen atom to which they are attached form a 5- to 7-membered ring;
m is zero or an integer from 1 to 5;
n is zero or an integer of 1 or 2;
or an optical isomer thereof; or a pharmaceutically acceptable salt thereof; or
(iii) Q is a 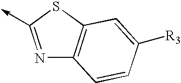
radical in which R₃ is hydrogen, halogen, alkyl, cycloalkyl, aryl, alkoxy, cycloalkoxy, aryloxy, alkylthio, cycloalkylthio, arylthio, acyl, sulfonyl, alkylamino, cycloalkylamino, arylamino, acylamino, sulfonamido or alkoxycarbonyl; and
R is a radical of the formula 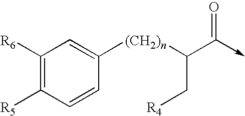
wherein
R₄ is C₂₋₄alkyl, C₃₋₇cycloalkyl or C₅₋₇heterocycloalkyl;
R₅ and R₆ are independently hydrogen, halogen, cyano, R₇, —C(O)R₇ or —S(O)₂R₇ wherein
R₇ is —(CR₈R₉)ₘ—W—R₁₀ in which
R₈ and R₉ are independently hydrogen or lower alkyl;
W is a bond, O, S or —NR₁₁ in which
R₁₁ is hydrogen or lower alkyl;
R₁₀ is hydrogen, alkyl, cycloalkyl, aryl or heterocyclyl; or R₁₀ and R₁₁, combined, are alkylene which together with the nitrogen atom to which they are attached form a 5- to 7-membered ring;
m is zero or an integer from 1 to 5;
n is zero or an integer of 1 or 2;
or an optical isomer thereof; or a pharmaceutically acceptable salt thereof; or
(iv) Q is a 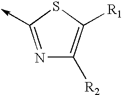
radical, wherein R₁ and R₂ are independently hydrogen or halogen; and
R is a radical of the formula 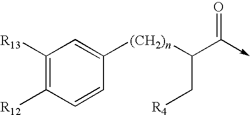
wherein
R₄ is C₂₋₄alkyl, C₃₋₇cycloalkyl or C₅₋₇heterocycloalkyl;
R₁₂ and R₁₃ are independently hydrogen, halogen, cyano, R₁₄, —C(O)R₁₄, or —S(O)₂R₁₄ wherein
R₁₄ is —(CR₈R₉)ₘ—W—R₁₅ in which
R₈ and R₉ are independently hydrogen or lower alkyl;
W is a bond, O, S or —NR₁₁ in which
R₁₁ is hydrogen or lower alkyl;
R₁₅ is cycloalkyl, aryl or heterocyclyl; or R₁₅ and R₁₁, combined, are alkylene which together with the nitrogen atom to which they are attached form a 5- to 7-membered ring;
m is zero or an integer from 1 to 5;
n is zero or an integer of 1 or 2;
or an optical isomer thereof; or a pharmaceutically acceptable salt thereof.

28. A method according to claim 22, wherein an inhibitor of stearoyl-CoA desaturase (SCD-1) is selected from the group consisting of
1-Pentyl-3-{6-[4-(2-trifluoromethylbenzoyl)piperazin-1-yl]pyridazin-3-yl}urea;
1-Benzyl-3-{6-[4-(2-trifluoromethylbenzoyl)piperazin-1-yl]pyridazin-3-yl}urea;
1-(4-Fluorophenyl)-3-{6-[4-(2-trifluoromethylbenzoyl)piperazin-1-yl]-pyridazin-3-yl}urea;
1-(2-Fluorophenyl)-3-{6-[4-(2-trifluoromethylbenzoyl)piperazin-1-yl]pyridazin-3-yl}urea;
1-[1-(4-Fluorophenyl)ethyl]-3-{6-[4-(2-trifluoromethylbenzoyl)-piperazin-1-yl]pyridazin-3-yl}urea;
1-[1-(4-Fluorophenyl)ethyl]-3-{6-[4-(2-trifluoromethylbenzoyl)piperazin-1-yl]pyridazin-3-yl}urea;
1-[3-(4-Fluorophenyl)propyl-3-{6-[4-(2-trifluoromethylbenzoyl)piperazin-1-yl]pyridazin-3-yl}urea;
1-Phenethyl-3-{6-[4-(2-trifluoromethylbenzoyl)piperazin-1-yl]-pyridazin-3-yl}urea;
1-(4-Fluorobenzyl)-3-{6-[4-(2-trifluoromethylbenzoyl)piperazin-1-yl]-pyridazin-3-yl}urea;
1-(3,4-Dichlorobenzyl)-3-{6-[4-(2-trifluoromethylbenzoyl)piperazin-1-yl]-pyridazin-3-yl}urea;
1-(2-Phenylcyclopropyl)-3-{6-[4-(2-trifluoromethylbenzoyl)piperazin-1-yl]-pyridazin-3-yl}urea;
1-Cyclopentyl-3-{6-[4-(2-trifluoromethylbenzoyl)piperazin-1-yl]-pyridazin-3-yl}urea;
1-(3-Cyclopropylpropyl)-3-{6-[4-(2-trifluoromethylbenzoyl)piperazin-1-yl]-pyridazin-3-yl}urea;
1-Cyclopropylmethyl-3-{6-[4-(2-trifluoromethylbenzoyl)piperazin-1-yl]-pyridazin-3-yl}urea;
1-(2-Cyclopropylethyl)-3-{6-[4-(2-trifluoromethylbenzoyl)-piperazin-1-yl]-pyridazin-3-yl}urea;
1-(2-Cyclopropylethyl)-3-{6-[4-(2-fluoro-6-trifluoromethylbenzoyl)piperazin-1-yl]-pyridazin-3-yl}urea;
1-(2-Cyclopropylethyl)-3-{6-[4-(5-fluoro-2-trifluoromethylbenzoyl)piperazin-1-yl]-pyridazin-3-yl}urea;
1-Cyclohexyl-3-{6-[4-(2-trifluoromethylbenzoy-l)piperazin-1-yl]-pyridazin-3-yl}urea;
1-(2-Cyclopropylethyl)-3-{6-[4-(2,6-difluorobenzoyl)piperazin-1-yl]-pyridazin-3-yl}urea;
1-(3-Cyclopropylpropyl)-3-{6-[4-(5-fluoro-2-trifluoromethylbenzoyl)piperazin-1-yl]-pyridazin-3-yl}urea;
4-Phenyl-N-{6-[4-(2-trifluoromethylbenzoyl)piperazin-1-yl]-pyridazin-3-yl}butyramide;
4-Methylpentanoic acid {6-[4-(2-trifluoromethylbenzoyl)-piperazin-1-yl]-pyridazin-3-yl}amide;
3-Cyclopentyl-N-{6-[4-(2-trifluoromethylbenzoyl)piperazin-1-yl]-pyridazin-3-yl}propionamide;
6-[4-(2-Trifluoromethylbenzoyl)piperazin-1-yl]-pyridazine-3-carboxylic acid phenethylamide;
6-[4-(2-Trifluoromethylbenzoyl)piperazin-1-yl]-pyridazine-3-carboxylic acid [2-(4-methoxyphenyl)ethyl]amide;
6-[4-(2-Trifluoromethylbenzoyl)piperazin-1-yl]-pyridazine-3-carboxylic acid [2-(3-fluorophenyl)ethyl]amide;
6-[4-(2-Trifluoromethylbenzoyl)piperazin-1-yl]-pyridazine-3-carboxylic acid (3-phenylpropyl)amide;
6-[4-(2-Trifluoromethylbenzoyl)piperazin-1-yl]-pyridazine-3-carboxylic acid [2-(4-fluorophenyl)ethyl]amide;
4-Methyl-2-({6-[4-(2-trifluoromethylbenzoyl)piperazin-1-yl]-pyridazine-3-carbonyl}amino)pentanoic acid methyl ester;
6-[4-(2-Trifluoromethylbenzoyl)piperazin-1-yl]-pyridazine-3-carboxylic acid (3-methyl butyl)amide;
6-[4-(5-Fluoro-2-trifluoromethylbenzoyl)piperazin-1-yl]-pyridazine-3-carboxylic acid (3-methylbutyl)amide;
6-[4-(4-Fluoro-2-trifluoromethylbenzoyl)piperazin-1-yl]-pyridazine-3-carboxylic acid (3-methylbutyl)amide;
4-Methyl-2-({6-[4-(2-trifluoromethylbenzoyl)piperazin-1-yl]-pyridazine-3-carbonyl}amino)pentanoic acid;
6-[4-(2-Trifluoromethylbenzoyl)piperazin-1-yl]-pyridazine-3-carboxylic acid (2-cyclopropylethyl)amide;
6-[4-(5-Fluoro-2-trifluoromethylbenzoyl)piperazin-1-yl]-pyridazine-3-carboxylic acid (2-cyclopropylethyl)amide;
6-[4-(2-Trifluoromethylbenzoyl)piperazin-1-yl]-pyridazine-3-carboxylic acid cyclopropylmethylamide;
4-(4-Methoxyphenyl)-N-{6-[4-(2-trifluoromethylbenzoyl)-piperazin-1-yl]-pyridazin-3-yl}butyramide;
3-(4-Fluorophenyl)-N-{6-[4-(2-trifluoromethylbenzoyl)-piperazin-1-yl]-pyridazin-3-yl}propionamide;
4-Cyclohexyl-N-{6-[4-(2-trifluoromethylbenzoyl)piperazin-1-yl]-pyridazin-3-yl}butyramide;
2,2,3,3-Tetramethylcyclopropanecarboxylic acid {6-[4-(2-trifluoromethylbenzoyl)piperazin-1-yl]pyridazin-3-yl}amide;
Cyclopropanecarboxylic acid {6-[4-(2-trifluoromethylbenzoyl)piperazin-1-yl]-pyridazin-3-yl}amide;
1-Trifluoromethylcyclopropanecarboxylic acid {6-[4-(2-trifluoromethylbenzoyl)piperazin-1-yl]-pyridazin-3-yl}amide;
2-Phenylcyclopropanecarboxylic acid {6-[4-(2-trifluoromethylbenzoyl)piperazin-1-yl]-pyridazin-3-yl}amide;
6-[4-(2-Trifluoromethylbenzoyl)piperazin-1-yl]-pyridazine-3-carboxylic acid indan-1-ylamide;
6-[4-(2-Trifluoromethylbenzoyl)piperazin-1-yl]-pyridazine-3-carboxylic acid (2-oxo-1,3-diaza-bicyclo[3.1.0]hex-3-en-4-yl)amide;
6-[4-(2-Trifluoromethylbenzoyl)piperazin-1-yl]-pyridazine-3-carboxylic acid (5-oxo-4,5-dihydro-1H-pyrazol-3-yl)amide;
6-[4-(2-Trifluoromethylbenzoyl)piperazin-1-yl]-pyridazine-3-carboxylic acid indan-5-ylamide;
5-[1,2]Dithiolan-3-yl-pentanoic acid {6-[4-(2-trifluoromethyl-benzoyl)-piperazin-1-yl]-pyridazin-3-yl}amide;
6-[4-(2-Trifluoromethyl-benzoyl)-piperazin-1-yl]-pyridazine-3-carboxylic acid (2-thiophen-2-yl-ethyl)amide;
6-[4-(2-Trifluoromethylbenzoyl)piperazin-1-yl]-pyridazine-3-carboxylic acid (2-benzo[1,3]dioxol-5-yl-ethyl)amide;
6-[4-(2-Trifluoromethylbenzoyl)piperazin-1-yl]-pyridazine-3-carboxylic acid (2,2-difluoro-2-pyridin-2-ylethyl)amide;
6-[4-(2-Trifluoromethylbenzoyl)piperazin-1-yl]-pyridazine-3-carboxylic acid (2-pyridin-2-ylethyl)amide;
6-[4-(2-Trifluoromethylbenzoyl)piperazin-1-yl]-pyridazine-3-carboxylic acid (pyridin-2-yl-methyl)amide;
6-[4-(2-Trifluoromethylbenzoyl)piperazin-1-yl]-pyridazine-3-carboxylic acid (5-chloropyridin-2-yl)amide;
6-[4-(2-Trifluoromethylbenzoyl)piperazin-1-yl]-pyridazine-3-carboxylic acid (5-trifluoromethylpyridin-2-yl)-amide;
6-[4-(2-Trifluoromethylbenzoyl)piperazin-1-yl]-pyridazine-3-carboxylic acid (7H-purin-6-yl)amide;
6-[4-(2-Trifluoromethylbenzoyl)piperazin-1-yl]-pyridazine-3-carboxylic acid pyrazin-2-ylamide;
6-[4-(2-Trifluoromethylbenzoyl)piperazin-1-yl]-pyridazine-3-carboxylic acid (1H-tetrazol-5-yl)amide;
6-[4-(2-Trifluoromethylbenzoyl)piperazin-1-yl]-pyridazine-3-carboxylic acid (2H-[1,2,4]triazol-3-yl)amide;
6-[4-(2-Trifluoromethylbenzoyl)piperazin-1-yl]-pyridazine-3-carboxylic acid (3-methylisoxazol-5-yl)amide;
6-[4-(2-Trifluoromethylbenzoyl)piperazin-1-yl]-pyridazine-3-carboxylic acid (5-methylisoxazol-3-yl)amide;
6-[4-(2-Trifluoromethylbenzoyl)piperazin-1-yl]-pyridazine-3-carboxylic acid (1H-pyrazol-3-yl)amide;
6-[4-(2-Trifluoromethylbenzoyl)piperazin-1-yl]-pyridazine-3-carboxylic acid (5-methyl-1H-pyrazol-3-yl)amide;
6-[4-(2-Trifluoromethylbenzoyl)piperazin-1-yl]-pyridazine-3-carboxylic acid pyrimidin-2-ylamide;
6-[4-(2-Trifluoromethylbenzoyl)piperazin-1-yl]-pyridazine-3-carboxylic acid pyrazin-2-ylamide;
6-[4-(2-Trifluoromethylben-zoyl)piperazin-1-yl]-pyridazine-3-carboxylic acid (4-methylpyrimidin-2-yl)amide;
6-[4-(2-Trifluoromethylbenzoyl)piperazin-1-yl]-pyridazine-3-carboxylic acid (2-oxo-2,3-dihydro-pyrimidin-4-yl)amide;
6-[4-(2-Trifluoromethylbenzoyl)piperazin-1-yl]-pyridazine-3-carboxylic acid (6-oxo-1,6-dihydro-pyrimidin-2-yl)amide;
6-[4-(2-Trifluoromethylbenzoyl)piperazin-1-yl]-pyridazine-3-carboxylic acid [1,3,4]thiadiazol-2-ylamide;
6-[4-(2-Trifluoromethylbenzoyl)piperazin-1-yl]-pyridazine-3-carboxylic acid thiazol-2-ylamide;
6-[4-(2-Trifluoromethylbenzoyl)piperazin-1-yl]-pyridazine-3-carboxylic acid pyridin-2-ylamide;
6-[4-(2-Trifluoromethylbenzoyl)piperazin-1-yl]-pyridazine-3-carboxylic acid pyridazin-3-ylamide;
6-[4-(2-Trifluoromethylbenzoyl)piperazin-1-yl]-pyridazine-3-carboxylic acid pyridin-3-ylamide;
6-[4-(2-Trifluoromethylbenzoyl)piperazin-1-yl]-pyridazine-3-carboxylic acid pyridin-4-ylamide;
6-[4-(2-Trifluoromethylbenzoy-l)piperazin-1-yl]-pyridazine-3-carboxylic acid (6-oxo-1,6-dihydro-[1,3,5]triazin-2-yl)amide;
6-[4-(2-Trifluoromethylbenzoyl)piperazin-1-yl]-pyridazine-3-carboxylic acid (5-fluoropyridin-2-yl)amide;
6-[4-(2-Trifluoromethylbenzoyl)-piperazin-1-yl]-pyridazine-3-carboxylic acid (5-cyanopyridin-2-yl)amide;
6-[4-(2-Trifluoromethylbenzoyl)-piperazin-1-yl]-pyridazine-3-carboxylic acid (4,6-dimethyl-pyrimidin-2-yl)amide;
6-[4-(2-Trifluoromethylbenzoyl)-piperazin-1-yl]-pyridazine-3-carboxylic acid (2-chloropyridin-4-yl)amide;
6-[4-(2-Trifluoromethylbenzoyl)-piperazin-1-yl]-pyridazine-3-carboxylic acid (1H-indol-6-yl)amide;
6-[4-(2-Trifluoromethylbenzoyl)-piperazin-1-yl]-pyridazine-3-carboxylic acid (1H-indol-4-yl)amide;
6-[4-(2-Trifluoromethylbenzoyl)-piperazin-1-yl]-pyridazine-3-carboxylic acid (1H-indazol-5-yl)amide;
6-[4-(2-Trifluoromethylbenzoyl)-piperazin-1-yl]-pyridazine-3-carboxylic acid (1H-indazol-6-yl)amide;
6-[4-(2-Trifluoromethylbenzoyl)-piperazin-1-yl]-pyridazine-3-carboxylic acid (4-methylthiazol-2-yl)amide;
6-[4-(2-Trifluoromethylbenzoyl)-piperazin-1-yl]-pyridazine-3-carboxylic acid (5-methylthiazol-2-yl)amide;
6-[4-(2-Trifluoromethylbenzoyl)-piperazin-1-yl]-pyridazine-3-carboxylic acid (5-thioxo-4,5-dihydro-1H-[1,2,4]triazol-3-yl)amide;
6-[4-(2-Trifluoromethylbenzoyl)piperazin-1-yl]-pyridazine-3-carboxylic acid (1H-benzoimidazol-2-yl)amide;
6-[4-(2-Trifluoromethylbenzoyl)piperazin-1-yl]-pyridazine-3-carboxylic acid (6-methylpyridazin-3-yl)amide;
6-[4-(2-Trifluoromethylbenzoyl)piperazin-1-yl]-pyridazine-3-carboxylic acid (6-methoxypyridazin-3-yl)amide;
6-[4-(2-Trifluoromethylbenzoyl)-piperazin-1-yl]-pyridazine-3-carboxylic acid (6-chloropyridazin-3-yl)amide;
6-[4-(2-Trifluoromethylbenzoyl)piperazin-1-yl]-pyridazine-3-carboxylic acid (4-chlorophenyl)amide;
6-[4-(2-Trifluoromethylbenzoyl)piperazin-1-yl]-pyridazine-3-carboxylic acid (4-carbamoyl-phenyl)amide;
6-[4-(2-Trifluoromethylbenzoyl)piperazin-1-yl]-pyridazine-3-carboxylic acid (3-carbamoyl-phenyl)amide;
6-[4-(2-Trifluoromethylbenzoyl)piperazin-1-yl]-pyridazine-3-carboxylic acid m-tolylamide;
6-[4-(2-Trifluoromethylbenzoyl)piperazin-1-yl]-pyridazine-3-carboxylic acid p-tolylamide;
6-[4-(2-Trifluoromethylbenzoyl)piperazin-1-yl]-pyridazine-3-carboxylic acid o-tolylamide;
6-[4-(2-Trifluoromethylbenzoyl)piperazin-1-yl]-pyridazine-3-carboxylic acid (2-propylphenyl)amide;
6-[4-(2-Trifluoromethylbenzoyl)piperazin-1-yl]-pyridazine-3-carboxylic acid (4-propylphenyl)amide;
6-[4-(2-Trifluoromethylbenzoyl)piperazin-1-yl]-pyridazine-3-carboxylic acid (4-isopropylphenyl)amide;
6-[4-(2-Trifluoromethylbenzoyl)piperazin-1-yl]-pyridazine-3-carboxylic acid (2-isopropylphenyl)amide;
6-[4-(2-Trifluoromethyl benzoyl)piperazin-1-yl]-pyridazine-3-carboxylic acid (2-chlorophenyl)amide;
6-[4-(2-Trifluoromethylbenzoyl)piperazin-1-yl]-pyridazine-3-carboxylic acid (2-cyano-3-fluorophenyl)amide;
6-[4-(2-Trifluoromethylbenzoyl)piperazin-1-yl]-pyridazine-3-carboxylic acid (2,4-dimethylphenyl)amide;
6-[4-(2-Trifluoromethyl benzoyl)piperazin-1-yl]-pyridazine-3-carboxylic acid (2,5-dimethylphenyl)amide;
6-[4-(2-Trifluoromethylbenzoyl)piperazin-1-yl]-pyridazine-3-carboxylic acid (2,6-dimethylphenyl)amide;
6-[4-(2-Trifluoromethylbenzoyl)piperazin-1-yl]-pyridazine-3-carboxylic acid (2,3-dimethylphenyl)amide;
6-[4-(2-Trifluoromethylbenzoyl)piperazin-1-yl]-pyridazine-3-carboxylic acid (3,5-dimethylphenyl)amide;
6-[4-(2-Trifluoromethylbenzoyl)piperazin-1-yl]-pyridazine-3-carboxylic acid (3,4-dimethylphenyl)amide;
6-[4-(2-Trifluoromethylbenzoyl)piperazin-1-yl]-pyridazine-3-carboxylic acid (4-ethyl phenyl)amide;
6-[4-(2-Trifluoromethylbenzoyl)piperazin-1-yl]-pyridazine-3-carboxylic acid (2-ethylphenyl)amide;
6-[4-(2-Trifluoromethylbenzoyl)piperazin-1-yl]-pyridazine-3-carboxylic acid (3-fluoro-2-methylphenyl)amide;
6-[4-(2-Trifluoromethylbenzoyl)piperazin-1-yl]-pyridazine-3-carboxylic acid (2-fluoro-4-methylphenyl)amide;
6-[4-(2-Trifluoromethylbenzoyl)piperazin-1-yl]-pyridazine-3-carboxylic acid (4-fluoro-2-methylphenyl)amide;
6-[4-(2-Trifluoromethylbenzoyl)piperazin-1-yl]-pyridazine-3-carboxylic acid (2-fluoro-5-methylphenyl)amide;
6-[4-(2-Trifluoromethylbenzoyl)piperazin-1-yl]-pyridazine-3-carboxylic acid (3-fluoro-5-methylphenyl)amide;
6-[4-(2-Trifluoromethylbenzoyl)piperazin-1-yl]-pyridazine-3-carboxylic acid (3-fluorophenyl)amide;
6-[4-(2-Trifluoromethylbenzoyl)piperazin-1-yl]-pyridazine-3-carboxylic acid (2-fluorophenyl)amide;
6-[4-(2-Trifluoromethylbenzoyl)piperazin-1-yl]-pyridazine-3-carboxylic acid (4-fluorophenyl)amide;
6-[4-(2-Trifluoromethylbenzoyl)piperazin-1-yl-pyridazine-3-carboxylic acid (2,4-difluorophenyl)amide;
6-[4-(2-Trifluoromethylbenzoyl)piperazin-1-yl]-pyridazine-3-carboxylic acid (2,5-difluorophenyl)amide;
6-[4-(2-Trifluoromethylbenzoyl)piperazin-1-yl]-pyridazine-3-carboxylic acid (3,4-difluorophenyl)amide;
6-[4-(2-Trifluoromethylbenzoyl)piperazin-1-yl]-pyridazine-3-carboxylic acid (2,3-difluorophenyl)amide;
6-[4-(2-Trifluoromethylbenzoyl)piperazin-1-yl]-pyridazine-3-carboxylic acid (2,6-difluorophenyl)amide;
6-[4-(2-Trifluoromethylbenzoyl)piperazin-1-yl]-pyridazine-3-carboxylic acid (4-cyanophenyl)amide;
6-[4-(2-Trifluoromethylbenzoyl)piperazin-1-yl]-pyridazine-3-carboxylic acid (2-cyanophenyl)amide;
6-[4-(2-Trifluoromethylbenzoyl)piperazin-1-yl]-pyridazine-3-carboxylic acid (3-cyanophenyl)amide;
6-[4-(2-Trifluoromethylbenzoyl)piperazin-1-yl]-pyridazine-3-carboxylic acid (3-chlorophenyl)amide;
6-[4-(2-Trifluoromethylbenzoyl)piperazin-1-yl]-pyridazine-3-carboxylic acid (3-chloro-2-methylphenyl)amide;
6-[4-(2-Trifluoromethylbenzoyl)piperazin-1-yl]-pyridazine-3-carboxylic acid (2-chloro-3-methylphenyl)amide;
6-[4-(2-Trifluoromethylbenzoyl)piperazin-1-yl]-pyridazine-3-carboxylic acid (2,5-dichlorophenyl)amide;
6-[4-(2-Trifluoromethylbenzoyl)piperazin-1-yl]-pyridazine-3-carboxylic acid (2-chloro-5-methylphenyl)amide;
6-[4-(2-Trifluoromethylbenzoyl)piperazin-1-yl]-pyridazine-3-carboxylic acid (2-chloro-6-methylphenyl)amide;
6-[4-(2-Trifluoromethylbenzoyl)piperazin-1-yl]-pyridazine-3-carboxylic acid (4-chloro-2-methylphenyl)amide;
6-[4-(2-Trifluoromethylbenzoyl)piperazin-1-yl]-pyridazine-3-carboxylic acid (4-chloro-3-methylphenyl)amide;
6-[4-(2-Trifluoromethylbenzoyl)piperazin-1-yl]-pyridazine-3-carboxylic acid (3-chloro-4-methylphenyl)amide;
6-[4-(2-Trifluoromethylbenzoyl)piperazin-1-yl]-pyridazine-3-carboxylic acid (2-chloro-4-methylphenyl)amide;
6-[4-(2-Trifluoromethylbenzoyl)piperazin-1-yl]-pyridazine-3-carboxylic acid (2-chloro-5-fluorophenyl)amide;
6-[4-(2-Trifluoromethylbenzoyl)piperazin-1-yl]-pyridazine-3-carboxylic acid (5-chloro-2-fluorophenyl)amide;
6-[4-(2-Trifluoromethylbenzoyl)piperazin-1-yl]-pyridazine-3-carboxylic acid (2,5-difluorophenyl)amide;
6-[4-(2-Trifluoromethylbenzoyl)piperazin-1-yl]-pyridazine-3-carboxylic acid (2,6-dichlorophenyl)amide;
6-[4-(2-Trifluoromethylbenzoyl)piperazin-1-yl]-pyridazine-3-carboxylic acid (2-trifluoromethylphenyl)amide;
6-[4-(2-Trifluoromethylbenzoyl)piperazin-1-yl]-pyridazine-3-carboxylic acid (4-trifluoromethylphenyl)amide;
6-[4-(2-Trifluoromethylbenzoyl)-piperazin-1-yl]-pyridazine-3-carboxylic acid (3-trifluoromethylphenyl)amide;
6-[4-(2-Trifluoromethylbenzoyl)piperazin-1-yl]-pyridazine-3-carboxylic acid phenylamide;
6-[4-(2-Trifluoromethylbenzoyl)piperazin-1-yl]-pyridazine-3-carboxylic acid (5-chloro-2-methoxyphenyl)amide;
6-[4-(2-Trifluoromethylbenzoyl)piperazin-1-yl]-pyridazine-3-carboxylic acid (2,5-dimethoxyphenyl)amide;
6-[4-(2-Trifluoromethylbenzoyl)piperazin-1-yl]-pyridazine-3-carboxylic acid (2-chloro-4-methoxyphenyl)amide;
6-[4-(2-Trifluoromethylbenzoyl)-piperazin-1-yl]-pyridazine-3-carboxylic acid (4-methoxyphenyl)amide;
6-[4-(2-Trifluoromethylbenzoyl)piperazin-1-yl]-pyridazine-3-carboxylic acid (2-methoxyphenyl)amide;
6-[4-(2-Trifluoromethylbenzoyl)piperazin-1-yl]-pyridazine-3-carboxylic acid (3-methoxyphenyl)amide;
4-({6-[4-(2-Trifluoromethylbenzoyl)piperazin-1-yl]-pyridazine-3-carbonyl}amino)-benzoic acid methyl ester;
4-({6-[4-(2-Trifluoromethylbenzoyl)piperazin-1-yl]-pyridazine-3-carbonyl}amino)-benzoic acid;
2-({6-[4-(2-Trifluoromethylbenzoyl)piperazin-1-yl]-pyridazine-3-carbonyl}amino)-benzoic acid methyl ester;
2-({6-[4-(2-Trifluoromethylbenzoyl)piperazin-1-yl]-pyridazine-3-carbonyl}amino)-benzoic acid;
6-[4-(2-Trifluoromethylbenzoyl)piperazin-1-yl]-pyridazine-3-carboxylic acid (3,4-dichlorophenyl)amide;
6-[4-(2-Trifluoromethylbenzoyl)piperazin-1-yl]-pyridazine-3-carboxylic acid [2-(2,4-fluorophenyl)ethyl]amide;
6-[4-(2-Trifluoromethylbenz-oyl)piperazin-1-yl]-pyridazine-3-carboxylic acid [2-(2-fluorophenyl)ethyl]amide;
6-[4-(2-Trifluoromethylbenzoyl)piperazin-1-yl]-pyridazine-3-carboxyli-c acid [2-(4-chlorophenyl)ethyl]amide;
6-[4-(2-Trifluoromethylbenzoyl)piperazin-1-yl]-pyridazine-3-carboxylic acid [2-(3-chlorophenyl)ethyl]amide;
6-[4-(2-Trifluoromethylbenzoyl)piperazin-1-yl]-pyridazine-3-carboxylic acid (2-phenylpropyl)amide;
6-[4-(2-Trifluoromethylbenzoyl)piperazin-1-yl]-pyridazine-3-carboxylic acid (2-biphenyl-4-ylethyl)amide;
(R)-6-[4-(2-Trifluoromethylbenzoyl)piperazin-1-yl]-pyridazine-3-carboxylic acid (2-hydroxy-2-phenylethyl)amide;
(S)-6-[4-(2-Trifluoromethylbenzoyl)-piperazin-1-yl]-pyridazine-3-carboxylic acid (2-hydroxy-2-phenylethyl)amide;
Acetic acid 1-phenyl-2-({6-[4-(2-trifluoromethyl-benzoyl)-piperazin-1-yl]-pyridazine-3-carbonyl}amino)ethyl ester;
6-[4-(2-Trifluoromethylbenzoyl)piperazin-1-yl]-pyridazine-3-carboxylic acid [3-(4-fluorophenyl)propyl]amide;
6-[4-(2-Trifluoromethylbenzoyl)piperazin-1-yl]-pyridazine-3-carboxylic acid (2,2-difluoro-2-phenylethyl)amide;
6-[4-(2-Trifluoromethylbenzoyl)piperazin-1-yl]-pyridazine-3-carboxylic acid [2-(3-fluorophenyl)-2-hydroxyethyl]amide;
6-[4-(2-Trifluoromethylbenzoyl)piperazin-1-yl]-pyridazine-3-carboxylic acid (4-hydroxybutyl)amide;
6-[4-(2-Trifluoromethylbenzoyl)piperazin-1-yl]-pyridazine-3-carboxylic acid (3-hydroxy-4,4-dimethylpentyl)amide;
6-[4-(2-Trifluoromethylbenzoyl)piperazin-1-yl]-pyridazine-3-carboxylic acid (3-hydroxy-3-methylbutyl)amide;
6-[4-(2-Trifluoromethylbenzoyl)piperazin-1-yl]-pyridazine-3-carboxylic acid (2-hydroxy-3,3-dimethylbutyl)amide;
6-[4-(5-Fluoro-2-trifluoromethylbenzoyl)piperazin-1-yl]-pyridazine-3-carboxylic acid (2-hydroxy-3,3-dimethylbutyl)amide;
6-[4-(2-Nitrobenzoyl)piperazin-1-yl]-pyridazine-3-carboxylic acid (3-methylbutyl)amide;
6-[4-(2-Chlorobenzoyl)piperazin-1-yl]-pyridazine-3-carboxylic acid (3-methylbutyl)amide;
6-[4-(2,4-Dichlorobenzoyl)piperazin-1-yl]-pyridazine-3-carboxylic acid (3-methylbutyl)amide;
6-[4-(2-Aminobenzoyl)piperazin-1-yl]-pyridazine-3-carboxylic acid (3-methyl butyl)amide;
6-[4-(2-Trifluoromethylbenzoyl)piperazin-1-yl]-pyridazine-3-carboxylic acid [2-(4-chlorophenoxy)ethyl]amide;
6-[4-(2-Trifluoromethyl benzoyl)piperazin-1-yl]-pyridazine-3-carboxylic acid [2-(4-fluorophenoxy)ethyl]amide;
6-[4-(2-Trifluoromethylbenzoyl)piperazin-1-yl]-pyridazine-3-carboxylic acid (3,3-dimethylbutyl)amide;
6-[4-(2-Trifluoromethylbenzoyl)piperazin-1-yl]-pyridazine-3-carboxylic acid pentylamide;
4-({6-[4-(2-Trifluoromethylbenzoyl)piperazin-1-yl]-pyridazine-3-carbonyl}amino)butyric acid ethyl ester;
6-[4-(5-Fluoro-2-trifluoromethylbenzoyl)piperazin-1-yl]-pyridazine-3-carboxylic acid pentylamide;
6-[4-(2-Trifluoromethylbenzoyl)piperazin-1-yl]-pyridazine-3-carboxylic acid (4-methylpentyl)amide;
6-[4-(2-Fluoro-6-trifluoromethylbenzoyl)piperazin-1-yl]-pyridazine-3-carboxylic acid (3-methylbutyl)amide;
6-[4-(2,6-Difluorobenzoyl)piperazin-1-yl]-pyridazine-3-carboxylic acid (3-methylbutyl)amide;
6-[4-(2-Trifluoromethylbenzoyl)piperazin-1-yl]-pyridazine-3-carboxylic acid (2-oxo-2-phenylethyl)amide;
Acetic acid 1,1-dimethyl-3-({6-[4-(2-trifluoromethyl-benzoyl)piperazin-1-yl]-pyridazine-3-carbonyl}amino)propyl ester;
6-[4-(2-Trifluoromethylbenzoyl)piperazin-1-yl]-pyridazine-3-carboxylic acid (2-phenoxyethyl)amide;
6-[4-(2-Trifluoromethylbenzoyl)piperazin-1-yl]-pyridazine-3-carboxylic acid hexylamide;
6-[4-(2-Trifluoromethylbenzoyl)piperazin-1-yl]-pyridazine-3-carboxylic acid (4-methylpentyl)amide;
6-[4-(5-Fluoro-2-trifluoromethylbenzoyl)piperazin-1-yl]-pyridazine-3-carboxylic acid (4-methylpentyl)amide;
6-[2,5-Dimethyl-4-(2-trifluoromethylbenzoyl)piperazin-1-yl]-pyridazine-3-carboxylic acid pentylamide;
6-[4-(2-Trifluoromethylbenzoyl)-piperazin-1-yl]-pyridazine-3-carboxylic acid heptylamide;
6-[4-(2-Sulfamoylbenzoyl)-piperazin-1-yl]-pyridazine-3-carboxylic acid (3-methylbutyl)-amide;
6-[4-(5-Chloro-2-trifluoromethyl-benzoyl)-piperazin-1-yl]-pyridazine-3-carboxylic acid hexylamide;
6-[4-(2-Trifluoromethylbenzoyl)-piperazin-1-yl]-pyridazine-3-carboxylic acid (2-cyclopropyl-2-oxoethyl)amide;
4-Trifluoromethyl-6-[4-(2-trifluoromethylbenzoyl)-piperazin-1-yl]-pyridazine-3-carboxylic acid (3-methyl-butyl)amide;
6-[4-(5-Fluoro-2-trifluoromethylbenzoyl)piperazin-1-yl]-pyridazine-3-carboxylic acid pentyl-4-enylamide;
6-(4-Benzoylpiperazin-1-yl)-pyridazine-3-carboxylic acid (2-cyclopropylethyl)amide;
6-[4-(2-Chloro-5-fluorobenzoyl)piperazin-1-yl]-pyridazine-3-carboxylic acid (2-cyclopropylethyl)amide;
6-[4-(5-Chloro-2-trifluoromethylbenzoyl)piperazin-1-yl]-pyridazine-3-carboxylic acid (2-cyclopropylethyl)amide;
6-[4-(2,6-Difluorobenzoyl)piperazin-1-yl]-pyridazine-3-carboxylic acid (2-cyclopropylethyl)amide;
6-[4-(2,5-Bis-trifluoromethylbenzoyl)piperazin-1-yl]-pyridazine-3-carboxylic acid (2-cyclopropylethyl)amide;
6-[4-(2,4-Bis-trifluoromethylbenzoyl)piperazin-1-yl]-pyridazine-3-carboxylic acid (2-cyclopropylethyl)amide;
6-[4-(2,5-Difluorobenzoyl)piperazin-1-yl]-pyridazine-3-carboxylic acid (2-cyclopropylethyl)amide;
6-[4-(5-Fluoro-2-trifluoromethylbenzoyl)piperazin-1-yl]-pyridazine-3-carboxylic acid (3-cyclopropylpropyl)amide;
6-[4-(2-Fluorobenzoyl)piperazin-1-yl]-pyridazine-3-carboxylic acid (2-cyclopropylethyl)amide;
6-[4-(3-Fluoro-2-trifluoromethylbenzoyl)piperazin-1-yl]-pyridazine-3-carboxylic acid (2-cyclopropylethyl)amide;
6-[4-(4-Fluoro-2-trifluoromethylbenzoyl)piperazin-1-yl]-pyridazine-3-carboxylic acid (3-cyclopropylpropyl)amide;
6-[4-(2-Trifluoromethylbenzoyl)piperazin-1-yl]-pyridazine-3-carboxylic acid (2-methylcyclopropylmethyl)amide;
6-[4-(5-Fluoro-2-methoxybenzoyl)piperazin-1-yl]-pyridazine-3-carboxylic acid (2-cyclopropylethyl)amide;
6-[4-(2-Dimethylaminobenzoyl)piperazin-1-yl]-pyridazine-3-carboxylic acid (2-cyclopropylethyl)amide;
6-[4-(2-Chloro-5-dimethylaminobenzoyl)piperazin-1-yl]-pyridazine-3-carboxylic acid (2-cyclopropylethyl)amide;
6-[4-(2,5-Dimethylbenzoyl)piperazin-1-yl]-pyridazine-3-carboxylic acid (2-cyclopropylethyl)amide;
6-[4-(2,5-Dichlorobenzoyl)piperazin-1-yl]-pyridazine-3-carboxylic acid (2-cyclopropylethyl)amide;
6-[4-(2-Trifluoromethylbenzoyl)piperazin-1-yl]-pyridazine-3-carboxylic acid cyclobutylmethylamide;
Acetic acid 2-{4-[6-(2-cyclopropylethylcarbamoyl)-pyridazin-3-yl]-piperazine-1-carbonyl}phenyl ester;
6-[4-(2-Trifluoromethylbenzoyl)piperazin-1-yl]-pyridazine-3-carboxylic acid (2-cyclopropyl-2-hydroxyethyl)amide;
6-[4-(2-Trifluoromethylbenzoyl)piperazin-1-yl]-pyridazine-3-carboxylic acid (2-phenylcyclopropylmethyl)amide;
6-[4-(2-Trifluoromethylbenzoyl)piperazin-1-yl]-pyridazine-3-carboxylic acid (3-cyclopropylpropyl)amide;
6-[4-(2-Cyanobenzoyl)piperazin-1-yl]-pyridazine-3-carboxylic acid (2-cyclopropylethyl)amide;
6-{4-[2-(2-Trifluoromethylphenyl)acetyl]-piperazin-1-yl}pyridazine-3-carboxylic acid (2-cyclopropylethyl)amide;
6-[4-(4-Fluoro-2-trifluoromethylbenzoyl)piperazin-1-yl]-pyridazine-3-carboxylic acid (2-cyclopropylethyl)amide;
6-[4-(5-Chloro-2-trifluoromethylbenzoyl)piperazin-1-yl]-pyridazine-3-carboxylic acid (3-cyclopropylpropyl)amide;
6-[3,5-Dimethyl-4-(2-trifluoromethylbenzoyl)piperazin-1-yl]-pyridazine-3-carboxylic acid (2-cyclopropylethyl)amide;
2-{4-[6-(2-Cyclopropylethylcarbamoyl)pyridazin-3-yl]-piperazine-1-carbonyl}benzoic acid methyl ester;
6-[4-(2-Trifluoromethyl-benzoyl)-piperazin-1-yl]-pyridazine-3-carboxylic acid (2-cyclobutylethyl)amide;
2-{4-[6-(2-Cyclopropyl-ethylcarbamoyl)-pyridazin-3-yl]-piperazine-1-carbonyl}-benzoic acid;
6-[4-(5-Chloro-2-trifluoromethyl-benzoyl)-piperazin-1-yl]-pyridazine-3-carboxylic acid (2-cyclobutylethyl)amide;
6-[4-(5-Fluoro-2-trifluoromethylbenzoyl)-piperazin-1-yl]-pyridazine-3-carboxylic acid (2-cyclobutylethyl)amide;
6-[4-(2-Trifluoromethylbenzoyl)-piperazin-1-yl]-pyridazine-3-carboxylic acid (3-cyclobutyl-propyl)amide;
6-[4-(5-Fluoro-2-trifluoromethylbenzoyl)-piperazin-1-yl]-pyridazine-3-carboxylic acid (2-cyclopropylethyl)amide;
6-[4-(2-Trifluoromethyl-thiobenzoyl)-piperazin-1-yl]-pyridazine-3-carboxylic acid (2-cyclopropylethyl)amide;
6-[4-(2-Trifluoromethylbenzoyl)-piperazin-1-yl]-pyridazine-3-carboxylic acid (4-cyclopropylbutyl)amide;
6-[4-(2-Trifluoromethylbenzoyl)-piperazin-1-yl]-pyridazine-3-carboxylic acid (2,2-dimethyl-cyclopropylmethyl)amide;
6-[4-(Pyridine-2-carbonyl)piperazin-1-yl]-pyridazine-3-carboxylic acid (2-cyclopropylethyl)amide;
6-[4-(2-Ttrifluoromethylfuran-3-carbonyl)piperazin-1-yl]-pyridazine-3-carboxylic acid (2-cyclopropylethyl)amide;
6-[4-(2-Chloro-4-trifluoromethylpyrimidine-5-carbonyl)piperazin-1-yl]-pyridazine-3-carboxylic acid (2-cyclopropylethyl)amide;
6-[4-(5-Methyl-2-trifluoromethylfuran-3-carbonyl)piperazin-1-yl]-pyridazine-3-carboxylic acid (2-cyclopropylethyl)amide;
6-[4-(2-Chloropyridine-3-carbonyl)piperazin-1-yl]-pyridazine-3-carboxylic acid (2-cyclopropylethyl)amide;
6-[4-(2-Methyl-5-trifluoromethyloxazole-4-carbonyl)piperazin-1-yl]-pyridazine-3-carboxylic acid (2-cyclopropylethyl)amide;
6-[4-(2,6-Dichloropyridine-3-carbonyl)piperazin-1-yl]-pyridazine-3-carboxylic acid (2-cyclopropylethyl)amide;
6-[4-(Pyrrolidine-1-carbonyl)piperazin-1-yl]-pyridazine-3-carboxylic acid (2-cyclopropylethyl)amide;
6-[4-(1-Methyl-1H-pyrrole-2-carbonyl)piperazin-1-yl]-pyridazine-3-carboxylic acid (2-cyclopropylethyl)amide;
6-[4-(Tetrahydrofuran-2-carbonyl)piperazin-1-yl]-pyridazine-3-carboxylic acid (2-cyclopropylethyl)amide;
6-{4-[2-(2-Trifluoromethylphenyl)acetyl]piperazin-1-yl}-pyridazine-3-carboxylic acid (2-cyclopropylethyl)amide;
4-[6-(3-Methylbutylcarbamoyl)pyridazin-3-yl]-piperazine-1-carboxylic acid t-butyl ester;
4-[6-(2-Cyclopropylethylcarbamoyl)pyridazin-3-yl]-piperazine-1-carboxylic acid t-butyl ester;
6-[4-(4,4,4-Trifluoro-3-hydroxy-3-trifluoromethylbutyryl)piperazin-1-yl]-pyridazine-3-carboxylic acid (2-cyclopropylethyl)amide;
6-[4-(4,4,4-Trifluoro-3-hydroxy-3-methylbutyryl)piperazin-1-yl]-pyridazine-3-carboxylic acid (2-cyclopropylethyl)amide;
and 6-[4-(3,3,3-Trifluoro-2-hydroxy-2-methylpropionyl)piperazin-1-yl]-pyridazine-3-carboxylic acid (2-cyclopropylethyl)amide;
6-[4-(1-Hydroxycyclopropanecarbonyl)piperazin-1-yl]-pyridazine-3-carboxylic acid (2-cyclopropylethyl)amide;
6-(4-Cyclobutanecarbonylpiperazin-1-yl)pyridazine-3-carboxylic acid (2-cyclopropylethyl)amide;
6-[4-(2-Trifluoromethylcyclopropanecarbonyl)piperazin-1-yl]-pyridazine-3-carboxylic acid (2-cyclopropylethyl)amide;
6-(4-Cyclohexanecarbonylpiperazin-1-yl)pyridazine-3-carboxylic acid (2-cyclopropylethyl)amide;
6-[4-(2-Methylcyclohexanecarbonyl)piperazin-1-yl]-pyridazine-3-carboxylic acid (2-cyclopropylethyl)amide;
6-[4-(3-Methylcyclohexanecarbonyl)piperazin-1-yl]-pyridazine-3-carboxylic acid (2-cyclopropylethyl)amide;
6-[4-(4-Methylcyclohexanecarbonyl)piperazin-1-yl]-pyridazine-3-carboxylic acid (2-cyclopropylethyl)amide;
6-[4-(2-Methylcyclopropanecarbonyl)piperazin-1-yl]-pyridazine-3-carboxylic acid (2-cyclopropylethyl)amide;
6-[4-(2,2,3,3-Tetramethylcyclopropanecarbonyl)piperazin-1-yl]pyridazine-3-carboxylic acid (2-cyclopropylethyl)amide;
6-[4-(2-Ethylbutyryl)piperazin-1-yl]-pyridazine-3-carboxylic acid (2-cyclopropylethyl)amide;
6-[4-(3,3,3-Trifluoro-2-methyl-2-trifluoromethylpropionyl)piperazin-1-yl]-pyridazine-3-carboxylic acid (2-cyclopropylethyl)amide;
6-[4-(2,2-Dimethylpropionyl)piperazin-1-yl]-pyridazine-3-carboxylic acid (2-cyclopropylethyl)amide;
6-[4-(2,2-Dimethylbutyryl)piperazin-1-yl]-pyridazine-3-carboxylic acid (2-cyclopropylethyl)amide;
6-[4-(2,2-Dimethylpentanoyl)piperazin-1-yl]-pyridazine-3-carboxylic acid (2-cyclopropylethyl)amide;
6-[4-(4,4,4-Trifluorobut-2-enoyl)piperazin-1-yl]-pyridazine-3-carboxylic acid (2-cyclopropylethyl)amide; and
6-[4-(4,4,4-Trifluoro-3-trifluoromethylbut-2-enoyl)-piperazin-1-yl]-pyridazine-3-carboxylic acid (2-cyclopropyl-ethyl)amide;
or a pharmaceutically acceptable salt thereof.

29. A method according to claim 22, wherein an inhibitor of protein tyrosine phosphatase (PTPase) is a compound of the formula 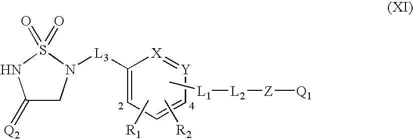
wherein
R₁ is hydrogen, halogen, hydroxy, alkoxy, carboxy, cyano, nitro, trifluoromethyl, alkynyl, alkylthio, heteroaralkyl, heteroaralkoxy or heteroaryloxy provided that R₁ is located at the 2-position when L₃ is —(CHR)ₛ— in which s is zero; or
R₁ is optionally substituted alkyl, alkenyl, optionally substituted amino, aralkyl, aralkoxy, aralkylthio, aryloxy, arylthio or cycloalkyl provided that a monocyclic aryl group which is substituted at the para position with a methylene or ethylene bridged nitrogen containing heterocycle does not constitute part of R₁ when
(i) R₁ is located at the 2-position and L₃ is —(CHR)ₛ— in which s is zero;
(ii) X and Y each are CH; and
(iii) Q₂ is oxygen; or
C—R₁ may be replaced with nitrogen or N→O; or
R₁ and R₂ combined together with the carbon atoms to which R₁ and R₂ are attached form an optionally substituted fused 5- to 6-membered aromatic or heteroaromatic ring provided that R₁ and R₂ are attached to carbon atoms adjacent to each other; or
R₂ is hydrogen, halogen, hydroxy, alkoxy, cyano, trifluoromethyl, nitro, optionally substituted amino, optionally substituted alkyl, alkylthio, aralkyl, heteroaralkyl, aralkoxy, heteroaralkoxy, aralkylthio, aryloxy, heteroaryloxy, arylthio or cycloalkyl; or
R₂ is —C(O)R₃ wherein
R₃ is hydroxy or optionally substituted alkoxy; or
R₃ is —NR₄R₅ in which R₄ and R₅ are independently hydrogen, optionally substituted alkyl, alkenyl, alkynyl, cycloalkyl, aryl, heterocyclyl, aralkyl or heteroaralkyl;
L₁ is a single bond; or
L₁ is carbon which combined together with R₂ and the carbon atoms to which L₁ and R₂ are attached form an optionally substituted fused 5- or 6-membered aromatic or heteroaromatic ring provided that L₁ and R₂ are attached to carbon atoms adjacent to each other; or
L₁ is CH or nitrogen which taken together with R₂ and the carbon atoms to which L₁ and R₂ are attached form a fused 5- to 7-membered ring which may be interrupted with one or two heteroatoms selected from oxygen, nitrogen and sulfur provided that L₁ and R₂ are attached to carbon atoms adjacent to each other; or
L₁ is CH, oxygen, sulfur or nitrogen and L₂ is carbon which combined together with L₁, R₂ and the carbon atoms to which L₁ and R₂ are attached form an optionally substituted fused 5- or 6-membered aromatic or heteroaromatic ring provided that L₁ and R₂ are attached to carbon atoms adjacent to each other; or
L₁ is —CH₂—, oxygen, sulfur or —NR₆— and L₂ is CH which taken together with L₁, R₂ and the carbon atoms to which L₁ and R₂ are attached form a fused 5- to 7-membered ring which may be interrupted with one or two heteroatoms selected from oxygen, nitrogen and sulfur wherein
R₆ is hydrogen, optionally substituted alkyl, aralkyl, heteroaralkyl, alkoxycarbonyl, aryloxycarbonyl, carbamoyl, sulfonyl or acyl provided that L₁ and R₂ are attached to carbon atoms adjacent to each other;
L₂ is —(CHR₇)ₙ— wherein
R₇ is hydrogen, hydroxy, alkoxy, carboxy, optionally substituted alkyl, cycloalkyl, aryl or heteroaryl;
n is zero or an integer from 1 to 4;
Z is —(CHR₈)ₘ—, —(CH₂)ₘO(CHR₈)ᵣ—, —(CH₂)ₘS(CHR₈)ᵣ— or —(CH₂)ₘNR₉(CHR₈)ᵣ— wherein
R₈ is hydrogen, optionally substituted alkyl, cycloalkyl, aryl or heterocyclyl;
R₉ is hydrogen, optionally substituted alkyl, cycloalkyl, aryl, heterocyclyl, aralkyl, heteroaralkyl, alkoxycarbonyl, aryloxycarbonyl, heteroaryloxycarbonyl, carbamoyl, sulfonyl, acyl or acylamino;
m and r are independently zero or an integer of 1 or 2;
Q₁ is hydrogen, optionally substituted alkyl, cycloalkyl, aryl or heterocyclyl provided that
(i) Q₁ is not 2-phenyloxazol-4-yl when
R₁ and R₂ are hydrogen;
X and Y each are CH;
L₁ is a single bond located at the 4-position;
L₂ is —(CHR₇)ₙ— wherein n is zero;
L₃ is —(CHR)ₛ— wherein s is zero;
Z is —(CH₂)ₘO(CHR₈)ᵣ— wherein R₈ is hydrogen, m is zero and r is 2; and
Q₂ is oxygen; or
(ii) Q₁ is not hydrogen when
R₁ and R₂ are hydrogen;
X and Y each are CH;
L₁ is a single bond;
L₂ is —(CHR₇)ₙ— wherein n is zero;
L₃ is —(CHR)ₛ— wherein R is hydrogen and s is 1;
Z is —(CHR₈)ₘ— wherein m is zero; and
Q₂ is oxygen; or
Q₁ is —C(O)NR₄ₐR₅ₐ, —C(O)R₁₀, —C(O)OR₁₀ or —S(O)_{q}R₁₀ wherein R₄ₐ and R₅ₐ are as defined for R₄ and R₅; R₁₀ is optionally substituted alkyl, cycloalkyl, aryl, heterocyclyl, aralkyl or heteroaralkyl; q is an integer of 1 or 2; or
Q₁ is a radical of the formula 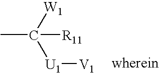
W₁ is aryl, heteroaryl, aralkyl or heteroaralkyl; or
W₁ is —C(O)R₃ₐ in which R₃ₐ is hydroxy or optionally substituted alkoxy; or
R₃ₐ is —NR₄ₐR₅ₐ in which R₄ₐ and R₅ₐ are as defined for R₄ and R₅;
R₁₁ is hydrogen, alkyl or aryl;
U₁ is —C(O)—, —S(O)₂— or —(CH₂)ᵣ— in which r is as defined for Z;
V₁ is hydroxy, alkoxy, aryl, heteroaryl, optionally substituted alkyl or cycloalkyl; or
V₁ is —NR_{4b}R_{5b} in which R_{4b} and R_{5b} are as defined for R₄ and R₅ provided that
(i) L₂ is —(CHR₇)ₙ— in which n is an integer of 1 or 2; and
(ii) Z is —(CHR₈)ₘ— in which m is zero; or
Q₁ is a radical of the formula 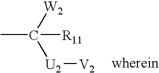
W₂ is —C(O)R₃ₐ in which R₃ₐ is hydroxy or optionally substituted alkoxy; or
R₃ₐ is —NR₄ₐR_{5b} in which R₄, and R₅, are as defined for R₄ and R₅;
R₁₁ is hydrogen, alkyl or aryl;
U₂ is —(CH₂)ₚ— in which p is zero or 1;
V₂ is —NR_{4b}C(O)R_{5b}, —NR_{4b}C(O)OR_{5b}, —NR_{4b}C(O)NR_{4c}R_{5b} or —NR_{4b}S(O)₂R_{5b} in which
R_{4b} and R_{4c} are as defined for R₄, and R_{5b} has a meaning as defined for R₅ provided that
(i) L₂ is —(CHR₇)ₙ— in which n is an integer of 1 or 2; and
(ii) Z is —(CHR₈)ₘ— in which m is zero; or
Q₁ is a radical of the formula 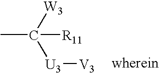
W₃ is —C(O)R₃ₐ in which R₃ₐ is hydroxy or optionally substituted alkoxy; or
R₃, is —NR₄ₐR₅ₐ in which R₄ₐ and R₅ₐ are as defined for R₄ and R₅;
R₁₁ is hydrogen, alkyl or aryl;
U₃ is —(CH₂)ₚ— in which p is zero or 1;
V₃ is —NHC(O)CHR_{4b}NHC(O)R₁₂ wherein R_{4b} is as defined for R₄; R₁₂ is hydrogen, aryl, heterocyclyl, aralkyl, heteroaralkyl, optionally substituted alkyl, alkoxy or cycloalkyl; or
R₁₂ is —NR_{4c}R_{5b}, in which R_{4c} and R_{5b} are as defined for R₄ and R₅ provided that
(i) L₂ is —(CHR₇)ₙ— in which n is an integer of 1 or 2; and
(ii) Z is —(CHR₈)ₘ— in which m is zero;
L₃ is —(CHR)ₛ— wherein
R is hydrogen, carboxy, optionally substituted alkyl, cycloalkyl, aryl or heteroaryl;
s is zero or an integer from 1 to 3;
Q₂ is oxygen, sulfur or NR₁₃ wherein
R₁₃ is hydrogen, hydroxy or lower alkyl;
X and Y are independently CH or nitrogen; or
—X═Y— is sulfur, oxygen or —NR₁₄— wherein
R₁₄ is hydrogen, optionally substituted alkyl, alkoxycarbonyl, acyl, aryloxycarbonyl, heteroaryloxycarbonyl, carbamoyl or sulfonyl;
or a pharmaceutically acceptable salt thereof; or a prodrug derivative thereof.

30-38. (canceled)
